(19) United States
(12) Patent Application Publication
(10) Pub. No.: US 2005/0176727 A1
(43) Pub. Date: Aug. 11, 2005

(54) Oximes and hydrazones that are useful in treating sexual dysfunction

(76) Inventors: Teodzyj Kolasa, Lake Villa, IL (US); Meena Patel, Green Oaks, IL (US); Kathleen H. Mortell, Chicago, IL (US); Mark A. Matulenko, Libertyville, IL (US); Ahmed A. Hakeem, Niles, IL (US); Pramila A. Bhatia, (US); Xueqing Wang, Evanston, IL (US); Jerome F. Daanen, Racine, WI (US); Steven P. Latshaw, Round Lake Beach, IL (US); Andrew O. Stewart, Libertyville, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/773,035

(22) Filed: Feb. 5, 2004

(51) Int. Cl. A61K 31/496; C07D 43/04

(52) U.S. Cl. 514/253.01; 544/360

(57) ABSTRACT

The present invention relates to oximes and hydrazones of formula (I) 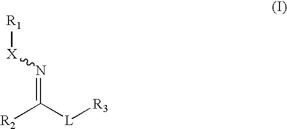 for the treatment of sexual dysfunction and to compositions containing compounds of formula (I) for the treatment of sexual dysfunction.

### TECHNICAL FIELD

The present invention relates to the use of oximes and hydrazones and compositions containing these compounds for the treatment of sexual dysfunction.

### BACKGROUND OF THE INVENTION

Preclinical evidence indicates that dopamine (DA) plays a role in penile erection in mammals. Sexual stimulation can be initiated by sensory (erotic) information reaching the cerebral cortex in mammals. The cerebral cortex has extensive neuronal connections with limbic structures like the amygdala, as well as midbrain structures like the periaqueductal gray (PAG) and the hypothalamus. Two important nuclei in the hypothalamus are the medial preoptic area (MPOA) and the paraventricular nucleus (PVN). The MPOA and PVN nuclei play a critical role in sexual behavior as bilateral lesions of these areas completely eliminate male sexual behavior. The incerto-hypothalamic dopaminergic pathway that innervates the PVN and the MPOA nuclei has been associated with the pro-erectile effect of DA agents. Systemic administration of DA receptor agonists like apomorphine ((6aR) 5,6,6a,7-tetrahydro-6-methyl-4H-dibenzo[de,g]quinoline-10,11-diol), quinpirole and (−) 3-(3-hydroxyphenyl)-N-propylpiperidine (3-PPP) facilitate penile erection in rats, an effect blocked by haloperidol, a central DA antagonist. As the erectogenic effect can not be blocked by domperidone, a peripheral DA antagonist, it is believed that the pro-erectile effect of DA agonists is centrally mediated (Andersson K and Wagner G, Physiol. Rev Vol. 75, pages 191-236 (1995); deGroat W and Booth A, in: Nervous control of urogenital system, Vol. 3, (ed. Maggi, C) pages 467-524, Hardwood Academic Publishers, Chur, Switzerland (1993); and Moreland R B, Nakane M, Hsieh G and Brioni J D, Curr. Opinion CPNS Invest. Drugs Vol. 2, pages 283-302 (2000)).

Clinical data also indicates that DA systems in the CNS play a role on the regulation of male sexual behavior as indicated by the sexual stimulatory effect of L-dopa in Parkinson's patients and by the pro-erectile effect of apomorphine in humans (Morales A, Geaton J, Johnston B and Adams M, Oral and Topical Treatment of Erectile Dysfunction: present and future, in: Urologic Clinics of North America Vol. 22, pages 879-886 (1995); Padma-Nathan H, Auerbach S, Lewis R, Lewand M and Perdok R, Urology Vol. 61 page 214 (abstract 821) (1999); and Dula E, Keating W, Siami P, Edmonds A, O'Neil J, Urology Vol. 56. pages 130-135 (2000)).

DA receptors belong to a superfamily of protein receptors that signal across the cell membrane by coupling to intracellular GTP-binding proteins. Several G proteins have been identified (including Gs, Gq and Gi) that lead to specific intracellular events (Milligan G and Rees S, Trends Pharmacol. Sci. Vol. 20, pages 118-124 (1999)).

There are five known DA receptors, which are classified into two groups, D₁-like and D₂-like. The D₁-like receptors include D₁ and D₅. The D₂-like receptors include D₂, D₃ and D₄ (Missale C, Nash S, Robinson S, Jaber M and Caron M, Physiol. Rev. Vol. 78 pages 189-225 (1998)). The D₁-like family receptor subtypes are Gₛ-coupled and can activate adenylate cyclase. The D₂-like family receptor subtypes are Gᵢ-coupled and they increase intracellular calcium level and inhibit adenylate cyclase.

The D₁-like family members are Gₛ-coupled receptors that can activate adenylate cyclase. The D₁ receptor is the most abundant and widespread DA receptor in the CNS both by mRNA expression and by immunohistochemical studies (Vallone D, Picetti R and Borreli E, Neurosci. Biobehav. Rev. Vol. 24, pages 125-132 (2000)). It is found in the striatum, nucleus accumbens and olfactory tubercle as well as the limbic system, hypothalamus and thalamus. The D₁ receptor expression has been reported in the heart and kidney, and despite that the function of these peripheral D₁ receptors remains to be clarified, its role on the control of hemodynamic variables has been confirmed. The D₅ receptor, while having a higher affinity for DA than the D₁ receptor, is sparsely distributed in the CNS with no evidence of expression outside the CNS.

The D₂-like family members are Gᵢ coupled receptors that inhibit adenylate cyclase and increase intracellular calcium levels. The D₂ receptor is the most abundant of the D₂-like receptors and is located in brain areas such as the striatum and substantia nigra, and in peripheral areas such as the heart, pituitary gland and kidney. The D₃ receptor is found abundantly in the islands of Calleja with distinct cluster populations in the ventral striatum/nucleus accumbens regions, olfactory tubercle, dentate gyrus and striatal cortex (Suzuki M, Hurd Y, Sokoloff P, Schwartz J and Sedwall G, Brain Res. Vol. 779, pages 58-74 (1998)).

Expression of the D₄ receptor has been documented by in situ RNA hybridization and immunohistochemical studies. Recently, studies revealed that D₄ expression is highest in the entorhinal cortex, lateral septal nucleus, hippocampus and the medial preoptic area of the hypothalamus (Primus R, Thurkauf A, Xu J, Yevich E, Mcinerney S, Shaw K, Tallman J and Gallagher D, J. Pharmacol. Exp. Ther. Vol. 282, pages 1020-1027 (1997)). Localization of D₄ is distinct from the distribution of D₂ in the brain, as D₂ receptors are most abundant in striatal areas. The expression of D₄ receptors in the MPOA of the hypothalamus is of importance to the facilitation of penile erection in view of the role of the hypothalamus as an area of integration between the cortex and the spinal pathways. The participation of D₄ receptors in other CNS regions, thalamic, subthalamic and spinal cannot be excluded.

British patent No. 1,378,080 to Buzas discloses oxime derivatives of halophenyl piperazinyl 3-methylene ketones as analgesic and anti-inflammatory agents. British patent No. 1,384,523 to Buzas discloses oxime derivatives halophenyl piperazinyl alkyl ketones as anti-inflammatory agents. Martinz-Esparza et al., disclose oximes with dual action at serotonin 5-HT_{1A} receptor and serotonin transporter potentially useful as antidepressant.

The present invention identifies a therapeutic use for oximes and hydrazones of formula (I-III) in the treatment of sexual dysfunction in mammals. More specifically, these compounds are useful in the treatment of sexual dysfunction including, but not limited to, male erectile dysfunction (MED).

### SUMMARY OF THE INVENTION

The present invention relates to oximes and hydrazones of formula (I) 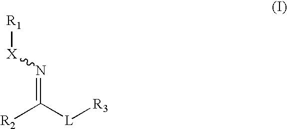
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 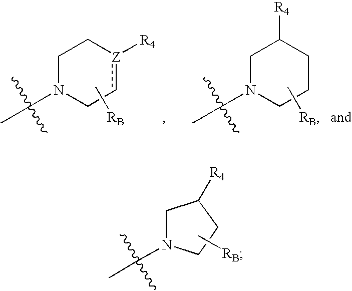
R₄ is heteroaryl;
L is C₁-C₂ alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C, CH, and N; and
— is absent or a single bond provided that when — is a single bond then Z is C.

### DETAILED DESCRIPTION OF THE INVENTION

All patents, patent applications, and literature references cited in the specification are herein incorporated by reference in their entirety.

In one embodiment, the present invention relates to oximes and hydrazones of formula (I) 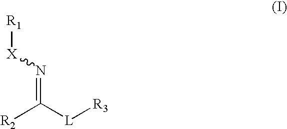
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 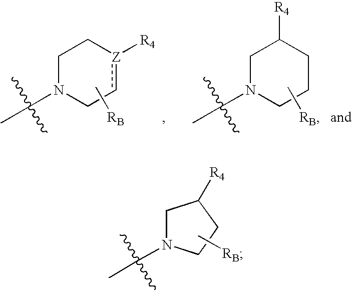
R₄ is heteroaryl;
L is C₁-C₂ alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C, CH, and N; and
— is absent or a single bond provided that when — is a single bond then Z is C.

In another embodiment, the present invention relates to compounds of formula (I) wherein R₃ is 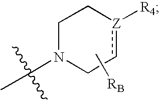
R₄ is heteroaryl; and R₁, R₂, R_{B}, X, Z, L, and — are as defined in formula (I).

In another embodiment, the present invention relates to compounds of formula (I) wherein X is O; R₂ is aryl; R₃ is 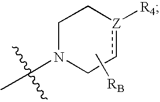
Z is N; — is absent; R₄ is heteroaryl; and R₁, R_{B}, and L are as defined in formula (I).

In another embodiment, the present invention relates to compounds of formula (I) wherein X is O; R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 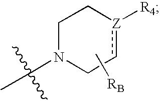
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, and L are as defined in formula (I).

In another embodiment, the present invention relates to compounds of formula (I) wherein X is O; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 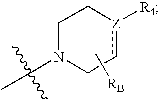
Z is N; — is absent; R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 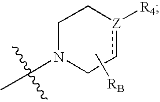
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is defined as in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 3-cyanophenyl, 2,4-dichlorophenyl, 3,4-dichlorophenyl, 3,5-difluorophenyl, 3-fluorophenyl, 4-fluorophenyl, 3,4-dimethylphenyl, 3,5-dimethylphenyl, 2-methylphenyl, and 3-methylphenyl; R₃ is 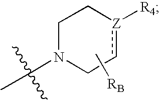
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; R₂ is arylalkyl; R₃ is 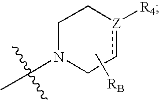
Z is N; — is absent; R₄ is heteroaryl; and R₁, R_{B}, and L, are as defined in formula (I).

In another embodiment, the present invention relates to compounds of formula (I) wherein X is O; R₂ is benzyl; R₃ is 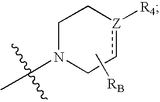
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, and L, are as defined in formula (I).

In another embodiment, the present invention relates to compounds of formula (I) wherein X is O; L is —CH₂—; R₁ is alkyl; R₂ is benzyl; R₃ is 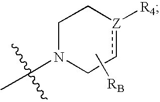
Z is N; — is absent; R₄ is pyridin-2-yl; and R_{B} is selected from the group consisting of hydrogen and methyl.

In another embodiment, the present invention relates to compounds of formula (I) wherein X is O; R₂ is heteroaryl; R₃ is 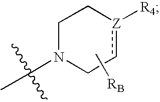
Z is N; — is absent; R₄ is heteroaryl; and R₁, R_{B}, and L, are as defined in formula (I).

In another embodiment, the present invention relates to compounds of formula (I) wherein X is O; L is —CH₂CH₂—; R₁ is alkyl; R₂ is pyridin-3-yl; R₃ is 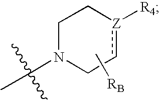
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; R₂ is aryl; R₃ is 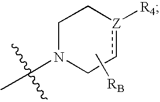
Z is C; — is a single bond; R₄ is heteroaryl; and R₁, R_{B}, and L, are as defined in formula (I).

In another embodiment, the present invention relates to compounds of formula (I) wherein X is O; R₂ is selected from the group consisting of naphthyl and phenyl wherein the phenyl is substituted with 0, 1, or 2 substituents independently selected from the group consisting of alkyl, cyano, and halogen; R₃ is 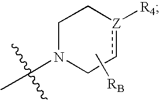
Z is C; — is a single bond; R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, and L, are as defined in formula (I).

In another embodiment, the present invention relates to compounds of formula (I) wherein X is O; L is selected defined as in formula (I); R₁ is selected from the group consisting of hydrogen, alkenyl, alkyl, cyanoalkyl, haloalkyl, and hydroxyalkyl; R₂ is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 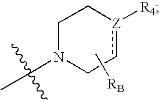
Z is C; — is a bond; R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 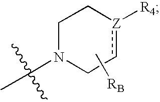
Z is C; — is a bond; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is selected from the group consisting of —CH₂— and —CH₂CH₂—; R₁ is methyl; R₂ is selected from the group consisting of 4-chlorophenyl, and 4-fluorophenyl; R₃ is 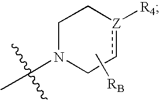
Z is C; — is a bond; R₄ is selected from the group consisting of 3-methylpyridin-2-yl and thiazol-2-yl; and R_{B} is selected from the group consisting of hydrogen and methyl.

In another embodiment, the present invention relates to compounds of formula (I) wherein X is O; R₂ is aryl; R₃ is 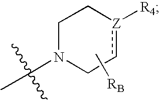
Z is CH; — is absent; R₄ is heteroaryl; and R₁, R_{B}, and L, are as defined in formula (I).

In another embodiment, the present invention relates to compounds of formula (I) wherein X is O; R₂ is selected from the group consisting of naphthyl and phenyl wherein the phenyl is substituted with 0, 1, or 2 substituents independently selected from the group consisting of alkyl, cyano, and halogen; R₃ is 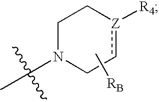
Z is CH; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, and L, are as defined in formula (I).

In another embodiment, the present invention relates to compounds of formula (I) wherein X is O; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 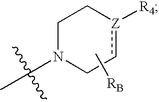
Z is CH; — is absent; R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 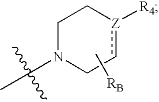
Z is CH; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is selected from the group consisting of —CH₂— and —CH₂CH₂—; R₁ is methyl; R₂ is selected from the group consisting of 4-chlorophenyl and 4-fluorophenyl; R₃ is 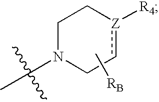
Z is CH; — is absent; R₄ is selected from the group consisting of pyridin-2-yl and 2-pyridinium N-oxide; and R_{B} is selected from the group consisting of hydrogen and methyl.

In another embodiment, the present invention relates to compounds of formula (I) wherein X is NR_{A}; R₂ is aryl; R₃ is 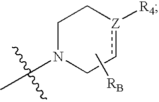
Z is N; — is absent; R₄ is heteroaryl; and R₁, R_{A}, R_{B}, and L, are as defined in formula (I).

In another embodiment, the present invention relates to compounds of formula (I) wherein X is NR_{A}; R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 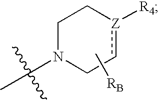
Z is N; — is absent; R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{A}, R_{B}, and L, are as defined in formula (I).

In another embodiment, the present invention relates to compounds of formula (I) wherein X is NR_{A}; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 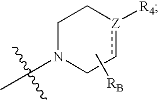
Z is N; — is absent; R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; R_{A} is selected from the group consisting of hydrogen and methyl; and R_{B} is selected from the group consisting of hydrogen and methyl.

In another embodiment, the present invention relates to compounds of formula (I) wherein X is NR_{A}; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 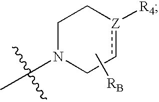
Z is N; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; R_{A} is selected from the group consisting of hydrogen and methyl; and R_{B} is selected from the group consisting of hydrogen and methyl.

In another embodiment, the present invention relates to compounds of formula (I) wherein X is NR_{A}; L is —CH₂—; R₁ is methyl; R₂ is 4-fluorophenyl; R₃ is 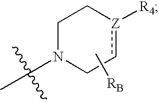
Z is N; — is absent; R₄ is selected from the group consisting of pyridin-2-yl; R_{A} is selected from the group consisting of hydrogen and methyl; and R_{B} is selected from the group consisting of hydrogen and methyl.

In another embodiment, the present invention relates to compounds of formula (I) wherein X is NR_{A}; R₂ is aryl; R₃ is 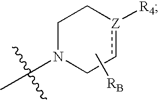
Z is CH; — is absent; R₄ is heteroaryl; and R₁, R_{A}, R_{B}, and L, are as defined in formula (I).

In another embodiment, the present invention relates to compounds of formula (I) wherein X is NR_{A}; R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 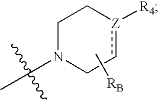
Z is CH; — is absent; R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{A}, R_{B}, and L, are as defined in formula (I).

In another embodiment, the present invention relates to compounds of formula (I) wherein X is NR_{A}; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 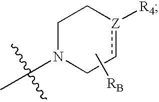
Z is CH; — is absent; R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; R_{A} is selected from the group consisting of hydrogen and methyl; and R_{B} is selected from the group consisting of hydrogen and methyl.

In another embodiment, the present invention relates to compounds of formula (I) wherein X is NR_{A}; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 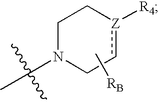
Z is CH; — is absent; R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; R_{A} is selected from the group consisting of hydrogen and methyl; and R_{B} is selected from the group consisting of hydrogen and methyl.

In another embodiment, the present invention relates to compounds of formula (I) wherein X is NR_{A}; L is —CH₂—; R₁ is methyl; R₂ is 4-fluorophenyl; R₃ is 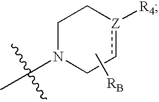
Z is CH; — is absent; R₄ is selected from the group consisting of pyridin-2-yl; R_{A} is selected from the group consisting of hydrogen and methyl; and R_{B} is selected from the group consisting of hydrogen and methyl.

In another embodiment, the present invention relates to compounds of formula (I) wherein R₃ is 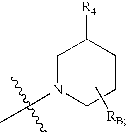
and R₁, R₂, R₄, R_{B}, X, and L are as defined in formula (I).

In another embodiment, the present invention relates to compounds of formula (I) wherein R₂ is aryl; R₃ is 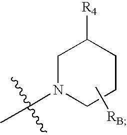
R₄is heteroaryl; and R₁, R_{B}, X, and L, are as defined in formula (I).

In another embodiment, the present invention relates to compounds of formula (I) wherein R₂ is selected from the group consisting of naphthyl and phenyl wherein the phenyl is substituted with 0, 1, or 2 substituents independently selected from the group consisting of alkyl, cyano, and halogen; R₃ is 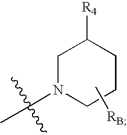
R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, X, and L, are as defined in formula (I).

In another embodiment, the present invention relates to compounds of formula (I) wherein X is O; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 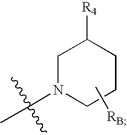
R₄ is is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is as defined in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 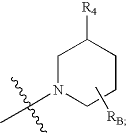
R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is selected from the group consisting of —CH₂— and —CH₂CH₂—; R₁ is selected from the group consisting of methyl and ethyl; R₂ is selected from the group consisting of phenyl, 4-chlorophenyl, and 4-fluorophenyl; R₃ is 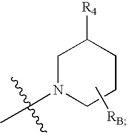
R₄ is selected from the group consisting of pyridin-2-yl, 2-pyridinium N-oxide, and thiazol-2-yl; and R_{B} is selected from the group consisting of hydrogen and methyl.

In another embodiment, the present invention relates to compounds of formula (I) wherein R₃ is 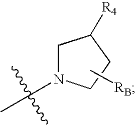
and R₁, R₂, R₄, R_{B}, X, and L are as defined in formula (I).

In another embodiment, the present invention relates to compounds of formula (I) wherein R₂ is aryl; R₃ is 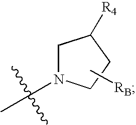
R₄ is heteroaryl; and R₁, R_{B}, X, and L are as defined in formula (I).

In another embodiment, the present invention relates to compounds of formula (I) wherein R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 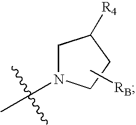
R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R₁, R_{B}, X, and L are as defined in formula (I).

In another embodiment, the present invention relates to compounds of formula (I) wherein X is O; L is as defined in formula (I); R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 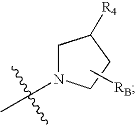
R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is defined a in formula (I); R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 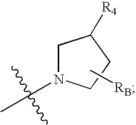
R₄ is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl; and R_{B} is selected from the group consisting of hydrogen and methyl.

In another embodiment, the present invention relates to compounds of formula (I) wherein X is O; L is selected from the group consisting of —CH₂— and —CH₂CH₂—; R₁ is selected from the group consisting of hydrogen and methyl; R₂ is 4-fluorophenyl; R₃ is 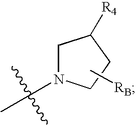
R₄ is pyrazin-2-yl; and R_{B} is selected from the group consisting of hydrogen and methyl.

In another embodiment, the present invention relates to a pharmaceutical composition comprising a therapeutically effective amount of a compound of formula (I) in combination with a pharmaceutically acceptable carrier.

In another embodiment, the present invention relates to a method of treating sexual dysfunction in a mammal comprising administering to the mammal a therapeutically effective amount of a compound of formula (I) or a pharmaceutically acceptable salt or prodrug thereof in combination with a pharmaceutically acceptable carrier.

In another embodiment, the present invention relates to a method of treating sexual dysfunction in a mammal comprising administering to the mammal a therapeutically effective amount of a compound of formula (I) or a pharmaceutically acceptable salt or prodrug thereof in combination with a phosphodiesterase 5 inhibitor.

In another embodiment, the present invention relates to a method of treating sexual dysfunction in a mammal comprising administering to the mammal a therapeutically effective amount of a compound of formula (I) or a pharmaceutically acceptable salt or prodrug thereof in combination with an adrenergic receptor antagonist.

In another embodiment, the present invention relates to a method of treating sexual dysfunction in a mammal comprising administering to the mammal a therapeutically effective amount of a compound of formula (I) or a pharmaceutically acceptable salt or prodrug thereof in combination with a dopamine agonist.

In another embodiment, the present invention relates to a method of treating male erectile dysfunction in a mammal comprising administering to the mammal in need of such treatment a therapeutically effective amount of a compound of formula (I) or a pharmaceutically acceptable salt or prodrug thereof.

In another embodiment, the present invention relates to a method of treating female sexual dysfunction in a mammal comprising administering to the mammal in need of such treatment a therapeutically effective amount of a compound of formula (I) or a pharmaceutically acceptable salt or prodrug thereof.

In another embodiment, the present invention relates to a method of treating cardiovascular disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (I) or a pharmaceutically acceptable salt or prodrug thereof.

In another embodiment, the present invention relates to a method of treating sexual dysfunction comprising administering a therapeutically effective amount of a compound of formula (Ia) 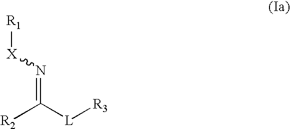
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 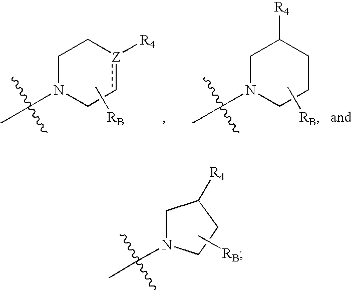
R₄ is heteroaryl;
L is alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C, CH, and N; and
— is absent or a single bond provided that when — is a single bond then Z is C;
   or a pharmaceutically acceptable salt or prodrug thereof in combination with a pharmaceutically acceptable carrier.

In another embodiment, the present invention relates to a method of treating sexual dysfunction comprising administering a therapeutically effective amount of a compound of formula (Ia) wherein R₃ is 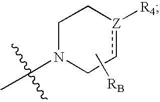
X is O;
R₂ is aryl;
Z is N;
— is absent; and
R₄ is heteroaryl.

In another embodiment, the present invention related to a method of treating sexual dysfunction comprising administering a therapeutically effective amount of a compound of formula (Ia), wherein X is O; R₂ is aryl wherein the aryl is selected from the group consisting of naphthyl and phenyl wherein the phenyl is substituted with 0, 1, or 2 substituents independently selected from the group consisting of alkyl, cyano, and halogen; Z is N; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is O; R₂ is arylalkyl; Z is N; — is absent; and
R₄ is heteroaryl.

In another embodiment, the present invention relates to a method of treating sexual dysfunction comprising administering a therapeutically effective amount of a compound of formula (Ia) wherein X is O; R₂ is arylalkyl wherein the arylalkyl is benzyl; Z is N; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is O; R₂ is heteroaryl; Z is N; — is absent; and R₄ is heteroaryl.

In another embodiment, the present invention relates to a method of treating sexual dysfunction comprising administering a therapeutically effective amount of a compound of formula (Ia) wherein X is O; R₂ is heteroaryl wherein the heteroaryl is pyridin-3-yl; Z is N; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is O; R₂ is aryl; Z is C; — is a single bond; and R₄ is heteroaryl.

In another embodiment, the present invention relates to a method of treating sexual dysfunction comprising administering a therapeutically effective amount of a compound of formula (Ia) wherein X is O; R₂ is aryl wherein the aryl is selected from the group consisting of naphthyl and phenyl wherein the phenyl is substituted with 0, 1, or 2 substituents independently selected from the group consisting of alkyl, cyano, and halogen; Z is C; — is a single bond; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is O; R₂ is aryl; Z is CH; — is absent; and R₄ is heteroaryl.

In another embodiment, the present invention relates to a method of treating sexual dysfunction comprising administering a therapeutically effective amount of a compound of formula (Ia) wherein X is O; R₂ is aryl wherein the aryl is selected from the group consisting of naphthyl and phenyl wherein the phenyl is substituted with 0, 1, or 2 substituents independently selected from the group consisting of alkyl, cyano, and halogen; Z is CH; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is NR_{A}; R₂ is aryl; Z is N; — is absent; and R₄ is heteroaryl.

In another embodiment, the present invention relates to a method of treating sexual dysfunction comprising administering a therapeutically effective amount of a compound of formula (Ia) wherein X is NR_{A}; R₂ is aryl wherein the aryl is selected from the group consisting of naphthyl and phenyl wherein the phenyl is substituted with 0, 1, or 2 substituents independently selected from the group consisting of alkyl, cyano, and halogen; Z is N; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein X is NR_{A}; R₂ is aryl; Z is CH; — is absent; and R₄ is heteroaryl.

In another embodiment, the present invention relates to a method of treating sexual dysfunction comprising administering a therapeutically effective amount of a compound of formula (Ia) wherein X is NR_{A}; R₂ is aryl wherein the aryl is selected from the group consisting of naphthyl and phenyl wherein the phenyl is substituted with 0, 1, or 2 substituents independently selected from the group consisting of alkyl, cyano, and halogen; Z is CH; — is absent; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein R₃ is 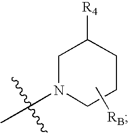
R₂ is aryl; and R₄ is heteroaryl.

In another embodiment, the present invention relates to a method of treating sexual dysfunction comprising administering a therapeutically effective amount of a compound of formula (Ia) wherein R₃ is 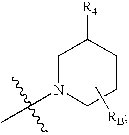
R₂ is aryl wherein the aryl is selected from the group consisting of naphthyl and phenyl wherein the phenyl is substituted with 0, 1, or 2 substituents independently selected from the group consisting of alkyl, cyano, and halogen; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a method of treating sexual dysfunction comprising administering a therapeutically effective amount of a compound of formula (Ia) wherein R₃ is 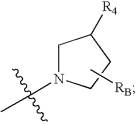
R₂ is aryl; and R₄ is heteroaryl.

In another embodiment, the present invention relates to a method of treating sexual dysfunction comprising administering a therapeutically effective amount of a compound of formula (Ia) wherein R₃ is 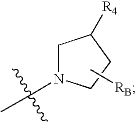
R₂ is aryl wherein the aryl is selected from the group consisting of naphthyl and phenyl wherein the phenyl is substituted with 0, 1, or 2 substituents independently selected from the group consisting of alkyl, cyano, and halogen; and R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

In another embodiment, the present invention relates to a pharmaceutical composition comprising a therapeutically effective amount of a compound of formula (Ia) in combination with a pharmaceutically acceptable carrier.

In another embodiment, the present invention relates to a method of treating sexual dysfunction in a mammal comprising administering to the mammal a therapeutically effective amount of a compound of formula (Ia) or a pharmaceutically acceptable salt or prodrug thereof in combination with a pharmaceutically acceptable carrier.

In another embodiment, the present invention relates to a method of treating sexual dysfunction in a mammal comprising administering to the mammal a therapeutically effective amount of a compound of formula (Ia) or a pharmaceutically acceptable salt or prodrug thereof in combination with a phosphodiesterase 5 inhibitor.

In another embodiment, the present invention relates to a method of treating sexual dysfunction in a mammal comprising administering to the mammal a therapeutically effective amount of a compound of formula (Ia) or a pharmaceutically acceptable salt or prodrug thereof in combination with an adrenergic receptor antagonist.

In another embodiment, the present invention relates to a method of treating sexual dysfunction in a mammal comprising administering to the mammal a therapeutically effective amount of a compound of formula (Ia) or a pharmaceutically acceptable salt or prodrug thereof in combination with a dopamine agonist.

In another embodiment, the present invention relates to a method of treating male erectile dysfunction in a mammal comprising administering to the mammal in need of such treatment a therapeutically effective amount of a compound of formula (Ia) or a pharmaceutically acceptable salt or prodrug thereof.

In another embodiment, the present invention relates to a method of treating female sexual dysfunction in a mammal comprising administering to the mammal in need of such treatment a therapeutically effective amount of a compound of formula (Ia) or a pharmaceutically acceptable salt or prodrug thereof.

In another embodiment, the present invention relates to a method of treating cardiovascular disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (Ia) or pharmaceutically acceptable salt or prodrug thereof. In another embodiment, the present invention relates to compounds of formula (II) 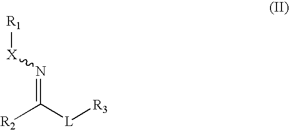
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 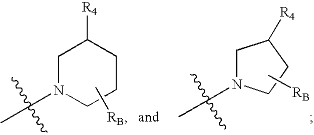
R₄ is aryl;
L is alkylene substituted with 0 or 1 substituent selected from the group consisting of alkoxy, alkoxyamino, hydroxy, and hydroxyiminoaryl; and
R_{B} is selected from the group consisting of hydrogen and alkyl.

In another embodiment, the present invention relates to compounds of formula (II) wherein R₃ is 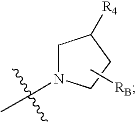
and R₁, R₂, R₄, X, L, and R_{B} are as defined in formula (II).

In another embodiment, the present invention relates to compounds of formula (II) wherein R₃ is 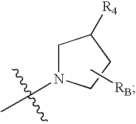
R₂ is aryl; R₄ is aryl; and R₁, X, L, and R_{B} are as defined in formula (II).

In another embodiment, the present invention relates to compounds of formula (II) wherein R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 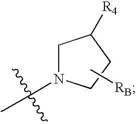
R₄ is aryl wherein the aryl is phenyl substituted with 0 or 1 substituent selected from the group consisting of alkoxy, cyano, and haloalkyl; and R₁, X, L, and R_{B} are as defined in formula (II).

In another embodiment, the present invention relates to compounds of formula (II) wherein X is O; L is selected from the group consisting of —CH₂—, —CH(CH₃)CH₂—, —CH(OH)CH₂—, —CH(OCH₃)CH₂—, —CH₂CH₂—, —CH₂CH₂CH₂—, —CH(CH₂CH₂C(═NOH)Ph)CH₂—, —CH(CH₂OCH(CH₃)₂)CH₂—, and —CH(CH₂NHOCH₃)CH₂—; R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 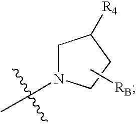
R₄ is aryl wherein the aryl is phenyl substituted with 0 or 1 substituent selected from the group consisting of alkoxy, cyano, and haloalkyl; and R_{B} is selected from the group consisting of hydrogen and methyl.

In another embodiment, the present invention relates to compounds of formula (II) wherein X is O; L is selected from the group consisting of —CH₂—, —CH(CH₃)CH₂—, —CH(OH)CH₂—, —CH(OCH₃)CH₂—, —CH₂CH₂—, —CH₂CH₂CH₂—, —CH(CH₂CH₂C(═NOH)Ph)CH₂—, —CH(CH₂OCH(CH₃)₂)CH₂—, and —CH(CH₂NHOCH₃)CH₂—; R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 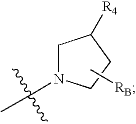
R₄ is selected from the group consisting of phenyl, 2-cyanophenyl, 2-ethoxyphenyl, 2-isopropoxyphenyl, 2-methoxyphenyl, 3-methoxyphenyl, 4-methoxyphenyl, and 3-trifluoromethylphenyl; and R_{B} is selected from the group consisting of hydrogen and methyl.

In another embodiment, the present invention relates to compounds of formula (II) wherein X is O; L is —CH₂CH₂—; R₁ is methyl; R₂ is 4-fluorophenyl; R₃ is 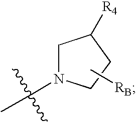
R₄ is selected from the group consisting of 2-methoxyphenyl, 3-methoxyphenyl, 4-methoxyphenyl, and 3-trifluoromethylphenyl; and R_{B} is selected from the group consisting of hydrogen and methyl.

In another embodiment, the present invention relates to compounds of formula (II) wherein R₃ is 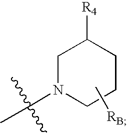
and R₁, R₂, R₄, X, L, and R_{B} are as defined in formula (II).

In another embodiment, the present invention relates to compounds of formula (II) wherein R₂ is aryl; R₃ is 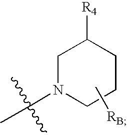
R₄ is aryl; and R₁, X, L, and R_{B} are as defined in formula (II).

In another embodiment, the present invention relates to compounds of formula (II) wherein R₂ is aryl wherein the aryl is selected from the group consisting of naphthyl and phenyl wherein the phenyl is substituted with 0, 1, or 2 substituents independently selected from the group consisting of alkyl, cyano, and halogen; R₃ is 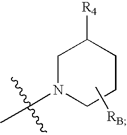
R₄ is aryl wherein the aryl is phenyl substituted with 0 or 1 substituent selected from the group consisting of alkoxy, cyano, and haloalkyl; and R₁, X, L, and R_{B} are as defined in formula (II).

In another embodiment, the present invention relates to compounds of formula (II) wherein X is O; L is selected from the group consisting of —CH₂—, —CH(CH₃)CH₂—, —CH(OH)CH₂—, —CH(OCH₃)CH₂—, —CH₂CH₂—, —CH₂CH₂CH₂—, —CH(CH₂CH₂C(═NOH)Ph)CH₂—, —CH(CH₂OCH(CH₃)₂)CH₂—, and —CH(CH₂NHOCH₃)CH₂—; R₁ is selected from the group consisting of hydrogen, alkenyl, alkyl, cyanoalkyl, haloalkyl, and hydroxyalkyl; R₂ is aryl wherein the aryl is selected from the group consisting of naphthyl and phenyl wherein the phenyl is substituted with 0, 1, or 2 substituents independently selected from the group consisting of alkoxy, alkenyl, alkyl, cyano, haloalkoxy, haloalkyl, halogen, hydroxy, hydroxyalkyl, and nitro; R₃ is 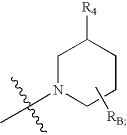
R₄ is aryl wherein the aryl is phenyl substituted with 0 or 1 substituent selected from the group consisting of alkoxy, cyano, and haloalkyl; and R_{B} is selected from the group consisting of hydrogen and methyl.

In another embodiment, the present invention relates to compounds of formula (II) wherein X is O; L is selected from the group consisting of —CH₂—, —CH(CH₃)CH₂—, —CH(OH)CH₂—, —CH(OCH₃)CH₂—, —CH₂CH₂—, —CH₂CH₂CH₂—, —CH(CH₂CH₂C(═NOH)Ph)CH₂—, —CH(CH₂OCH(CH₃)₂)CH₂—, and —CH(CH₂NHOCH₃)CH₂—; R₁ is selected from the group consisting of hydrogen, methyl, ethyl, propyl, butyl, isopropyl, allyl, cyanomethyl, 2-hydroxyethyl, and 2,2,2-trifluoroethyl; R₂ is selected from the group consisting of naphthyl, phenyl, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl; R₃ is 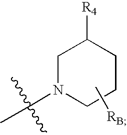
R₄ is selected from the group consisting of phenyl, 2-cyanophenyl, 2-ethoxyphenyl, 2-isopropoxyphenyl, 2-methoxyphenyl, 3-methoxyphenyl, 4-methoxyphenyl, and 3-trifluoromethylphenyl; and R_{B} is selected from the group consisting of hydrogen and methyl.

In another embodiment, the present invention relates to compounds of formula (II) wherein X is O; L is —CH₂CH₂—; R₁ is methyl; R₂ is selected from the group consisting of phenyl, 4-chlorophenyl, and 4-fluorophenyl; R₃ is 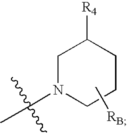
R₄ is phenyl; and R_{B} is selected from the group consisting of hydrogen and methyl.

In another embodiment, the present invention relates to a pharmaceutical composition comprising a therapeutically effective amount of a compound of formula (II) in combination with a pharmaceutically acceptable carrier.

In another embodiment, the present invention relates to a method of treating sexual dysfunction in a mammal comprising administering to the mammal a therapeutically effective amount of a compound of formula (II) or a pharmaceutically acceptable salt or prodrug thereof in combination with a pharmaceutically acceptable carrier.

In another embodiment, the present invention relates to a method of treating sexual dysfunction in a mammal comprising administering to the mammal a therapeutically effective amount of a compound of formula (II) or a pharmaceutically acceptable salt or prodrug thereof in combination with a phosphodiesterase 5 inhibitor.

In another embodiment, the present invention relates to a method of treating sexual dysfunction in a mammal comprising administering to the mammal a therapeutically effective amount of a compound of formula (II) or a pharmaceutically acceptable salt or prodrug thereof in combination with an adrenergic receptor antagonist.

In another embodiment, the present invention relates to a method of treating sexual dysfunction in a mammal comprising administering to the mammal a therapeutically effective amount of a compound of formula (II) or a pharmaceutically acceptable salt or prodrug thereof in combination with a dopamine agonist.

In another embodiment, the present invention relates to a method of treating male erectile dysfunction in a mammal comprising administering to the mammal in need of such treatment a therapeutically effective amount of a compound of formula (II) or a pharmaceutically acceptable salt or prodrug thereof.

In another embodiment, the present invention relates to a method of treating female sexual dysfunction in a mammal comprising administering to the mammal in need of such treatment a therapeutically effective amount of a compound of formula (II) or a pharmaceutically acceptable salt or prodrug thereof.

In another embodiment, the present invention relates to a method of treating cardiovascular disorders, inflammatory disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (II) or a pharmaceutically acceptable salt or prodrug thereof.

In another embodiment, the present invention relates to a method of treating sexual dysfunction in a mammal comprising administering to the mammal a therapeutically effective amount of a compound of formula (III) 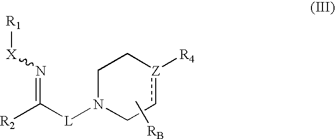
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₄ is aryl;
L is alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C and CH; and
— is absent or a single bond provided that when — is a single bond then Z is C.

In another embodiment, the present invention relates to a pharmaceutical composition comprising a therapeutically effective amount of a compound of formula (III) in combination with a pharmaceutically acceptable carrier.

In another embodiment, the present invention relates to a method of treating sexual dysfunction in a mammal comprising administering to the mammal a therapeutically effective amount of a compound of formula (III) or a pharmaceutically acceptable salt or prodrug thereof in combination with a pharmaceutically acceptable carrier.

In another embodiment, the present invention relates to a method of treating sexual dysfunction in a mammal comprising administering to the mammal a therapeutically effective amount of a compound of formula (III) or a pharmaceutically acceptable salt or prodrug thereof in combination with a phosphodiesterase 5 inhibitor.

In another embodiment, the present invention relates to a method of treating sexual dysfunction in a mammal comprising administering to the mammal a therapeutically effective amount of a compound of formula (III) or a pharmaceutically acceptable salt or prodrug thereof in combination with an adrenergic receptor antagonist.

In another embodiment, the present invention relates to a method of treating sexual dysfunction in a mammal comprising administering to the mammal a therapeutically effective amount of a compound of formula (III) or a pharmaceutically acceptable salt or prodrug thereof in combination with a dopamine agonist.

In another embodiment, the present invention relates to a method of treating male erectile dysfunction in a mammal comprising administering to the mammal in need of such treatment a therapeutically effective amount of a compound of formula (III) or a pharmaceutically acceptable salt or prodrug thereof.

In another embodiment, the present invention relates to a method of treating female sexual dysfunction in a mammal comprising administering to the mammal in need of such treatment a therapeutically effective amount of a compound of formula (III) or a pharmaceutically acceptable salt or prodrug thereof.

In another embodiment, the present invention relates to a method of treating cardiovascular disorders, inflammatory disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (III) or a pharmaceutically acceptable salt or prodrug thereof.

### DEFINITIONS OF THE PRESENT INVENTION

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, means an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl, and methoxymethyl.

The term “alkoxyamino” as used herein, means an alkoxy group, as defined herein, appended to the parent molecular moiety through an NH group. Representative examples of alkoxyamino include, but are not limited to, methoxyamino, ethoxyamino, and propoxyamino.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms that is substituted with 0 or 1 substituent selected from the group consisting of alkoxy, alkoxyamino, hydroxy, and hydroxyiminoaryl. Representative examples of alkylene include, but are not limited to, —CH₂—, —CH(CH₃)—, —C(CH₃)₂—, —CH(CH₃)CH₂—, —CH(OH)CH₂—, —CH(OCH₃)CH₂—, —CH₂CH₂—, —CH₂CH₂CH₂—, —CH₂CH₂CH₂CH₂—, —CH₂CH(CH₃)CH₂—, —CH(CH₂CH₂C(═NOH)Ph)CH₂—, —CH(CH₂OCH(CH₃)₂)CH₂—, and —CH(CH₂NHOCH₃)CH₂—.

The term “alkylthio” as used herein, means an alkyl group, as defined herein, appended to the parent molecular moiety through a sulfur atom. Representative examples of alkylthio include, but are not limited, methylthio, ethylthio, tert-butylthio, and hexylthio.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a phenyl or naphthyl group.

The aryl groups of this invention can be substituted with 0, 1, 2, 3, 4, or 5 substituents independently selected from alkenyl, alkoxy, alkoxyalkyl, alkoxycarbonyl, alkyl, alkylcarbonyl, alkylcarbonyloxy, alkylthio, alkynyl, carboxy, cyano, formyl, haloalkoxy, haloalkyl, halogen, hydroxy, hydroxyalkyl, mercapto, nitro, —NR_{C}R_{D} and (NR_{C}R_{D})carbonyl. Representative examples include, but are not limited to, 4-bromophenyl, 2-chlorophenyl, 3-chlorophenyl, 4-chlorophenyl, 4-chloro-3-methylphenyl, 3-chloro-4-fluorophenyl, 2-cyanophenyl, 3-cyanophenyl, 4-cyanophenyl, 2,4-dichlorophenyl, 3,4-dichlorophenyl, 3,5-dichlorophenyl, 2,4-difluorophenyl, 3,4-difluorophenyl, 3,5-difluorophenyl, 2-fluorophenyl, 3-fluorophenyl, 4-fluorophenyl, 2,4-dimethylphenyl, 3,4-dimethylphenyl, 3,5-dimethylphenyl, 3-methyl-5-chlorophenyl, 2-methylphenyl, 3-methylphenyl, and 4-methylphenyl.

The term “arylalkyl” as used herein, means an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “carbonyl” as used herein, means a —C(O)— group.

The term “carboxy” as used herein, means a —CO₂H group.

The term “cyano” as used herein, means a —CN group.

The term “cyanoalkyl” as used herein, means a cyano group, as defined herein, appended to the parent molecular moiety through an alkyl group, as defined herein. Representative examples of cyanoalkyl include, but are not limited to, cyanomethyl, 2-cyanoethyl, and 3-cyanopropyl.

The term “cycloalkyl” as used herein, means a saturated cyclic hydrocarbon group containing from 3 to 8 carbons, examples of cycloalkyl include cyclopropyl, cyclobutyl, cyclopentyl, cyclohexyl, cycloheptyl, and cyclooctyl.

The cycoalkyl groups of the present invention are optionally substituted with 1, 2, 3, or 4 substituents selected from alkenyl, alkoxy, alkoxyalkyl, alkoxycarbonyl, alkyl, alkylcarbonyl, alkylcarbonyloxy, alkylthio, alkynyl, carboxy, cyano, formyl, haloalkoxy, haloalkyl, halogen, hydroxy, hydroxyalkyl, mercapto, nitro, —NR_{C}R_{D} and (NR_{C}R_{D})carbonyl.

The term “formyl” as used herein, means a —C(O)H group.

The term “halo” or “halogen” as used herein, means —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heteroaryl,” as used herein, means an aromatic monocyclic ring or an aromatic bicyclic ring. The aromatic monocyclic rings are five or six membered rings containing 1, 2, 3, or 4 heteroatoms independently selected from the group consisting of N, O, and S. The nitrogen heteroatoms can be optionally quaternized or oxidized to the N-oxide. The nitrogen containing rings can be optionally N-protected. The five membered aromatic monocyclic rings have two double bonds and the six membered aromatic monocyclic rings have three double bonds. The aromatic bicyclic rings are composed of an aromatic monocyclic ring fused to a phenyl group. Alternatively, aromatic bicyclic rings are composed of an aromatic monocyclic ring fused to another aromatic monocyclic ring. The aromatic monocyclic rings and the aromatic bicyclic rings are connected to the parent molecular moiety through a carbon or nitrogen atom. Representative examples of heteroaryl include, but are not limited to, benzothienyl, benzoxadiazolyl, cinnolinyl, dibenzofuranyl, furopyridinyl, furyl, imidazolyl, indazolyl, indolyl, isoxazolyl, isoquinolinyl, isothiazolyl, naphthyridinyl, oxadiazolyl, oxazolyl, pyridinyl, pyridazinyl, pyrimidinyl, pyrazinyl, pyrazolyl, pyridinium N-oxide, pyrrolyl, quinolinyl, tetrazolyl, thiadiazolyl, thiazolyl, thienopyridinyl, thienyl, triazolyl, and triazinyl.

The heteroaryl groups of the present invention are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkynyl, carboxy, cyano, formyl, haloalkoxy, haloalkyl, halogen, hydroxy, hydroxyalkyl, mercapto, nitro, —NR_{C}R_{D} and (NR_{C}R_{D})carbonyl. Representative examples include, but are not limited to, 3-cyanopyridin-2-yl, 5-hydroxypyridin-2-yl, and 3-methylpyridin-2-yl.

The term “hydroxy” as used herein, means an —OH group.

The term “hydroxyalkyl” as used herein, means at least one hydroxy group, as defined herein, is appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypentyl, and 2-ethyl-4-hydroxyheptyl.

The term “hydroxyimino” as used herein, means a HON═C group.

The term “hydroxyiminoaryl” as used herein, means an aryl group, as defined herein, is appended to the parent molecular moiety through a hydroxyimino group. Representative examples of hydroxyiminoaryl include, but are not limited to, hydroxyiminophenyl, hydroxyimino-4-bromophenyl, hydroxyimino-2-chlorophenyl, hydroxyimino-3-chlorophenyl, hydroxyimino-4-chlorophenyl, hydroxyimino-3-chloro-4-fluorophenyl, hydroxyimino-2-cyanophenyl, hydroxyimino-3-cyanophenyl, hydroxyimino-4-cyanophenyl, hydroxyimino-2,4-dichlorophenyl, hydroxyimino-3,4-dichlorophenyl, hydroxyimino-3,5-dichlorophenyl, hydroxyimino-2,4-difluorophenyl, hydroxyimino-3,4-difluorophenyl, hydroxyimino-3,5-difluorophenyl, hydroxyimino-2-fluorophenyl, hydroxyimino-3-fluorophenyl, hydroxyimino-4-fluorophenyl, hydroxyimino-2,4-dimethylphenyl, hydroxyimino-3,4-dimethylphenyl, hydroxyimino-3,5-dimethylphenyl, hydroxyimino-3-methyl-5-chlorophenyl, hydroxyimino-2-methylphenyl, hydroxyimino-3-methylphenyl, and hydroxyimino-4-methylphenyl.

The term “mercapto” as used herein, means a —SH group.

The term “mercaptoalkyl” as used herein, means a mercapto group, as defined herein, appended to the parent molecular moiety through an alkyl group, as defined herein. Representative examples of mercaptoalkyl include, but are not limited to, 2-mercaptoethyl and 3-mercaptopropyl.

The term “nitro” as used herein, means a —NO₂ group.

The term “—NR_{C}R_{D}” as used herein, means two groups, R_{C} and R_{D}, which are appended to the parent molecular moiety through a nitrogen atom. R_{C} and R_{D} are each independently selected from the group consisting of hydrogen, alkyl, alkylcarbonyl, and formyl. Representative examples of —NR_{C}R_{D} include, but are not limited to, amino, methylamino, acetylamino, and acetylmethylamino.

The term “(NR_{C}R_{D})carbonyl” as used herein, means a —NR_{C}R_{D} group, as defined herein, appended to the parent molecular moiety through a carbonyl group, as defined herein. Representative examples of (NR_{C}R_{D})carbonyl include, but are not limited to, aminocarbonyl, (methylamino)carbonyl, (dimethylamino)carbonyl, and (ethylmethylamino)carbonyl.

The term “male sexual dysfunction” as used herein includes, but is not limited to, male erectile dysfunction or premature ejacualtion.

The term “female sexual dysfunction” as used herein includes, but is not limited to, female anorgasmia, clitoral erectile insufficiency, vaginal engorgement, dyspareunia, or vaginismus.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials that contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Compounds of the present invention were named by ACD/ChemSketch version 5.0 (developed by Advanced Chemistry Development, Inc., Toronto, ON, Canada) or were given names that appeared to be consistent with ACD nomenclature.

### Abbreviations

Abbreviations which have been used in the descriptions of the Schemes and the Examples that follow are: Ac for acetyl; n-Bu for n-butyl; DMF for N,N-dimethylformamide; DMSO for dimethylsulfoxide; Ph for phenyl; TFA for trifluoroacetic acid; and THF for tetrahydrofuran.

### PREPARATION OF COMPOUNDS OF THE PRESENT INVENTION

The compounds and processes of the present invention will be better understood in connection with the following synthetic Schemes and Examples, which illustrate a means by which the compounds of the present invention can be prepared.

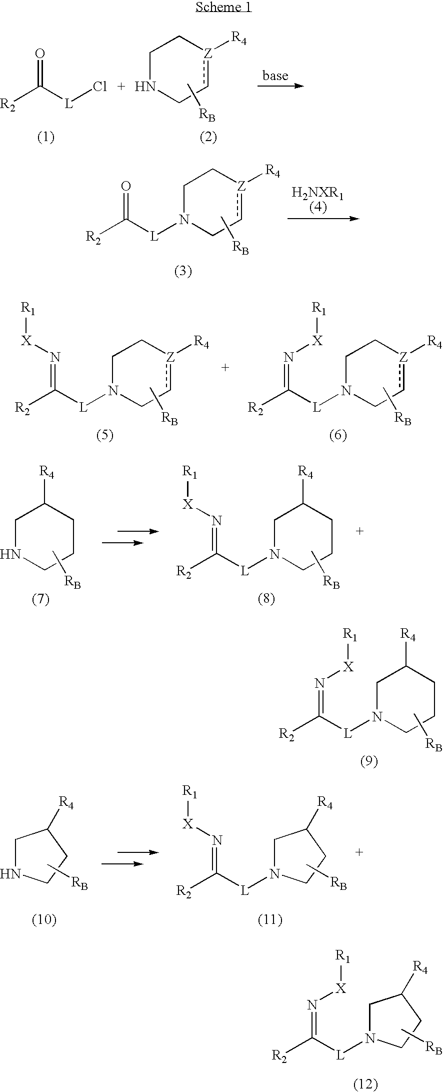

Compounds of the present invention, wherein R₁, R₂, R₄, R_{B}, X and L are as defined in formula (I), can be prepared as described in Scheme 1. Chloroketones (or bromoketones) of general formula (1) can be treated with piperidines, tetrahydropyridines, or piperazines of general formula (2) to provide ketones of general formula (3). Ketones of general formula (3) can be treated with hydroxy amines or hydrazines of general formula (4) to provide oximes or hydrazones of general formula (8) and (9). The (E) and (Z) isomers can generally be separated by chromatography.

Piperidines of general formula (7) can be treated in a similar manner to provide oximes or hydrazones of general formula (8) and (9). The (E) and (Z) isomers can generally be separated by chromatography.

Pyrrolidines of general formula (10) can be treated in a similar manner to provide oximes or hydrazones of general formula (11) and (12). The (E) and (Z) isomers can generally be separated by chromatography.

### General Procedure for Salt Formation

The following general procedure was used to prepare maleate salts of compounds of the present invention.

The free base (1 mmol) in methanol (10 mL) was treated with maleic acid (1 mmol), stirred for 5 minutes, and the mixture was concentrated under reduced pressure. The residue was azeotroped with ethanol (10 mL) and toluene (10 mL), dried under reduced pressure, treated with anhydrous diethyl ether and filtered. The filter cake was washed with diethyl ether and dried under reduced pressure to provide the desired salt.

### EXAMPLE 1

### (1E)-1-(3-chlorophenyl)-3-(4-pyridin-2-yl-piperazin-1-yl)propan-1-one O-methyloxime and

### (1Z)-1-(3-chlorophenyl)-3-(4-pyridin-2-ylpiperazin-1-yl)propan-1-one O-methyloxime

### EXAMPLE 1A

### 1-(3-chlorophenyl)-3-(4-pyridin-2-ylpiperazin-1-yl)propan-1-one

3-Chloroacetophenone (1.55 g, 10 mmol), 1-(2-pyridinyl)piperazine (1.1 mL, 7 mmol), paraformaldehyde (300 mg, 10 mmol), and concentrated HCl (2 mL, 23 mmol) were combined in isopropanol (20 mL) and refluxed for 16 hours. The mixture was allowed to cool to room temperature and concentrated under reduced pressure. The residue was treated with saturated NaHCO₃ and extracted with ethyl acetate. The organics were washed with brine, dried with anhydrous MgSO₄, filtered, and the filtrate was concentrated under reduced pressure. The residue was purified by column chromatography (silica gel, ethyl acetate) to provide the title compound. MS (DCI/NH₃) m/z 330 (M+H)⁺.

### EXAMPLE 1B

### (1E)-1-(3-chlorophenyl)-3-(4-pyridin-2-ylpiperazin-1-yl)propan-1-one O-methyloxime and

### (1Z)-1-(3-chlorophenyl)-3-(4-pyridin-2-ylpiperazin-1-yl)propan-1-one O-methyloxime

The product from Example 1A (329 mg, 1 mmol) and O-methylhydroxylamine hydrochloride (415 mg, 5 mmol) were combined in pyridine (20 mL) at room temperature and stirred for 12 hours. The mixture was concentrated under reduced pressure and saturated NaHCO₃ was added. The mixture was extracted with ethyl acetate. The organics were separated and washed with brine, dried with MgSO₄, filtered, and the filtrate was concentrated under reduced pressure. The residue was purified by column chromatography (silica gel, CH₂Cl₂:acetone 4:1) to afford the title compounds.

E-isomer: maleate salt, mp 170-172° C. ¹H NMR (300 MHz, DMSO-d₆) δ 3.20 (m, 12H), 3.98 (s, 3H), 6.08 (s, 2H), 6.73 (d-d, J=7 Hz, 4 Hz, 1H), 6.95(d, J=9 Hz, 1H), 7.50 (m, 2H), 7.62 (m, 2H), 7.73 (m, 1H), 8.16 (m, 1H); MS (DCI/NH₃) m/z 359 (M+H)⁺. Anal. calcd for C₂₃H₂₇ClN₄O₅: C, 58.16; H, 5.73; N, 11.80. Found: C, 57.91; H, 5.91; N, 11.55.

Z-isomer: maleate salt, mp 145-147° C. ¹H NMR (300 MHz, DMSO-d₆) δ 3.30 (m, 12H), 3.79 (s, 3H), 6.08 (s, 2H), 6.74 (d-d, J=7 Hz, 4 Hz, 1H), 6.94 (d, J=9 Hz, 1), 7.46 (m, 3H), 7.58 (m, 2H), 8.16 (m, 1H); MS (DCI/NH₃) m/z 359 (M+H)⁺. Anal. calcd for C₂₃H₂₇ClN₄O₅.0.4H₂O: C, 57.30; H, 5.81; N, 11.62. Found: C, 57.05; H, 5.64; N, 11.35.

### EXAMPLE 2

### (1E)-1-(4-chlorophenyl)-3-(4-pyridin-2-yl-piperazin-1-yl)propan-1-one O-methyloxime and

### (1Z)-1-(4-chlorophenyl)-3-(4-pyridin-2-ylpiperazin-1-yl)propan-1-one O-methyloxime

### EXAMPLE 2A

### 1-(4-chlorophenyl)-3-(4-pyridin-2-yl-piperazin-1-yl)propan-1-one

3,4′-Dichloropropiophenone (1.02 g, 5 mmol) and 1-(2-pyridinyl)piperazine (1.63 g, 10 mmol) were combined in toluene (35 mL) and refluxed for 8 hours. The mixture was concentrated in vacuo and the residue was purified by chromatography (silica gel, ethyl acetate) to provide the title compound. MS (DCI/NH₃) m/z 330 (M+H)⁺.

### EXAMPLE 2B

### (1E)-1-(4-chlorophenyl)-3-(4-pyridin-2-yl-piperazin-1-yl)propan-1-one O-methyloxime and

### (1Z)-1-(4-chlorophenyl)-3-(4-pyridin-2-ylpiperazin-1-yl)propan-1-one O-methyloxime

The title compounds were prepared using the procedure described in Example 1B except using the product from Example 2A instead of the product from Example 1A.

E-isomer: mp 67-68° C. ¹H NMR (300 MHz, DMSO-d₆) δ 2.45 (m, 6H), 2.93 (t, J=7 Hz, 2H), 3.42 (t, J=4.5 Hz, 4H), 3.93 (s, 3H), 6.62 (dd, J=4 Hz, 7 Hz, 1H), 6.80 (d, J=9 Hz, 1H), 7.50 (m, 3H), 7.68 (m, 2H), 8.10 (m, 1H); MS (DCI/NH₃) m/z 359 (M+H)⁺. Anal. calcd for C₁₉H₂₃ClN₄O.0.75H2O: C, 61.40; H, 6.51; N, 15.07. Found: C, 61.17; H, 6.73; N, 14.93.

Z-isomer: mp 61-64° C. ¹H NMR (300 MHz, DMSO-d₆) δ 2.40 (m, 6H), 2.70 (t, J=7 Hz, 2H), 3.42 (t, J=4.5 Hz, 4H), 3.72 (s, 3H), 6.62 (dd, J=4 Hz, 7 Hz, 1H), 6.80 (d, J=9 Hz, 1H), 7.45 (m, 5H), 8.10 (m, 1H); MS (DCI/NH₃) m/z 359 (M+H)⁺.

E-isomer: maleate salt, mp ° C. ¹H NMR (300 MHz, DMSO-d₆) δ 3.20 (m, 12H), 3.98 (s, 3H), 6.08 (s, 2H), 6.73 (d-d, J=7 Hz, 4 Hz, 1H), 6.95(d, J=9 Hz, 1H), 7.50 (m, 2H), 7.60 (m, 1H), 7.73 (m, 2H), 8.16 (m, 1H); MS (DCI/NH₃) m/z 359 (M+H)⁺. Anal. calcd for C₂₃H₂₇ClN₄O₅: C, 58.16; H, 5.73; N, 11.80. Found: C, 57.91; H, 5.91; N, 11.55.

Z-isomer: maleate salt, mp ° C. ¹H NMR (300 MHz, DMSO-d₆) δ 3.30 (m, 12H), 3.79 (s, 3H), 6.08 (s, 2H), 6.74 (d-d, J=7 Hz, 4 Hz, 1H), 6.94 (d, J=9 Hz, 1H), 7.46 (m, 3H), 7.58 (m, 2H), 8.16 (m, 1H); MS (DCI/NH₃) m/z 359 (M+H)⁺. Anal. calcd for C₂₃H₂₇ClN₄O₅.0.4H₂O: C, 57.30; H, 5.81; N, 11.62. Found: C, 57.05; H, 5.64; N, 11.35.

E-isomer: di-tartrate salt, ¹H NMR (300 MHz, DMSO-d₆) δ 2.30 (s, 0.75H), 2.55 (m, 5H), 2.95 (m, 2H), 3.46 (m, 5H), 3.92 (s, 3H), 4.28 (s, 4H), 6.63 (d-d, J=7 Hz, 4 Hz, 1H), 6.92(d, J=9 Hz, 1H), 7.20 (m, 1.3H), 7.48 (m, 3H), 7.68 (m, 2H), 8.10 (m, 1H); MS (DCI/NH₃) m/z 359 (M+H)⁺. Anal. calcd for C₂₇H₃₅ClN₄O₁₃.0.25C₇H₈.0.4H₂O: C, 50.12; H, 5.49; N, 8.13. Found: C, 50.00; H, 5.45; N, 7.73.

Z-isomer: di-tartrate salt, mp 145-147° C. ¹H NMR (300 MHz, DMSO-d₆) δ 2.54 (m, 6H), 2.74 (m, 2H), 3.45 (m, 4H), 3.73 (s, 3H), 4.28 (s, 4H), 6.63 (d-d, J=7 Hz, 4 Hz, 1H), 6.82 (d, J=9 Hz, 1H), 7.47 (m, 5H), 8.10 (m, 1H); MS (DCI/NH₃) m/z 359 (M+H)⁺. Anal. calcd for C₂₇H₃₅ClN₄O₁₃.1.2H₂O: C, 47.64; H, 5.54; N, 8.23. Found: C, 48.01; H, 5.45; N, 7.72.

### EXAMPLE 3

### (1E)-1-(4-fluorophenyl)-2-(4-pyridin-2-ylpiperazin-1-yl)ethanone O-methyloxime and

### (1Z)-1-(4-fluorophenyl)-2-(4-pyridin-2-ylpiperazin-1-yl)ethanone O-methyloxime

### EXAMPLE 3A

### 1-(4-fluorophenyl)-2-(4-pyridin-2-ylpiperazin-1-yl)ethanone

4-Fluorophenacyl chloride (860 mg, 5 mmol), 1-(2-pyridinyl)piperazine dihydrochloride (1420 mg, 6 mmol), and anhydrous K₂CO₃ (2760 mg, 20 mmol) were combined in DMF (30 mL) and stirred at room temperature for 6 hours. The mixture was treated with water and extracted with ethyl acetate. The organic layer was washed with water, brine, dried with MgSO₄, filtered, and the filtrate was concentrated under reduced pressure. The residue was chromatographed (silica gel, hexanes:ethyl acetate 1:1) to afford the title compound. ¹H NMR (300 MHz, DMSO-d₆) δ 2.60 (t, J=4 hz, 4H), 3.48 (t, J=4 Hz, 4H), 3.89 (s, 2H), 6.62 (dd, J=7 Hz, 4 Hz, 1H), 7.80 (d, J=9 Hz, 1H), 7.35 (t, J=9 Hz, 2H), 7.52 (m, 1H), 8.11 (m, 3H); MS (DCI/NH₃) m/z 345 (M+H)⁺.

### EXAMPLE 3B

### (1E)-1-(4-fluorophenyl)-2-(4-pyridin-2-ylpiperazin-1-yl)ethanone O-methyloxime and

### (1Z)-1-(4-fluorophenyl)-2-(4-pyridin-2-ylpiperazin-1-yl)ethanone O-methyloxime

The title compounds were prepared using the procedure described in Example 1B except using the product from Example 3A instead of the product from Example 1A.

Z-isomer: di-maleate salt, ¹H NMR (300 MHz, DMSO-d₆) δ 3.30 (m, 10H), 3.86 (s, 3H), 6.20 (s, 4H), 6.70 (d-d, J=7 Hz and 4 Hz, 1H), 6.88 (d, J=9 Hz, 1H), 7.30 (t, J=9 Hz, 2H), 7.60 (m, 1H), 7.68 (m, 2H), 8.12 (m, 1H); MS (DCI/NH₃) m/z 329 (M+H)⁺.

Anal. calcd for C₂₆H₂₉FN₄O₉.1.2H₂O: C, 53.61; H, 5.33; N, 9.62. Found: C, 53.24; H 5.11; N, 9.44.

E-isomer: di-maleate salt, ¹H NMR (300 MHz, DMSO-d₆) δ 3.30 (m, 10H), 3.97 (s, 3H), 6.20 (s, 4H), 6.70 (d-d, J=7 Hz and 4 Hz, 1H), 6.87 (d, J=9 Hz, 1H), 7.30 (t, J=9 Hz, 2H), 7.58 (m, 1H), 7.85 (m, 2H), 8.13 (m, 1H); MS (DCI/NH₃) m/z 329 (M+H)⁺.

Anal. calcd for C₂₆H₂₉FN₄O₉: C, 55.71; H, 5.21; N, 10.00. Found: C, 55.59; H, 5.33; N, 10.07.

### EXAMPLE 4

### (1E)-1-(4-chlorophenyl)-2-(4-pyridin-2-ylpiperazin-1-yl)ethanone O-methyloxime and

### (1Z)-1-(4-chlorophenyl)-2-(4-pyridin-2-ylpiperazin-1-yl)ethanone O-methyloxime

### EXAMPLE 4A

### 1-(4-chlorophenyl)-2-(4-pyridin-2-ylpiperazin-1-yl)ethanone

The title compound was prepared using the procedure described in Example 3A except using 4-chlorophenacyl chloride instead of 4-fluorophenacyl chloride.

### EXAMPLE 4B

### (1E)-1-(4-chlorophenyl)-2-(4-pyridin-2-ylpiperazin-1-yl)ethanone O-methyloxime and

### (1Z)-1-(4-chlorophenyl)-2-(4-pyridin-2-ylpiperazin-1-yl)ethanone O-methyloxime

The title compounds were prepared using the procedure described in Example 1B except using the product from Example 4A instead of the product from Example 1A.

Z-isomer: di-maleate salt, mp 153-154° C.; ¹H NMR (300 MHz, DMSO-d₆) δ 2.80 (m, 4H), 3.51 (m, 4H), 3.98 (m, 5H), 6.22 (s, 4H), 6.68 (d-d, J=7 Hz and 4 Hz, 1H), 6.85 (d, J=9 Hz, 1H), 7.50 (d, J=9 Hz, 2H), 7.57 (m, 1H), 7.81 (d, J=9 Hz, 2H), 8.09 (m, 1H); MS (DCI/NH₃) m/z 345 (M+H)⁺; Anal. calcd for C₂₆H₂₉ClN₄O₉.0.7H₂O: C, 52.97; H, 5.20; N, 9.50. Found: C, 52.73; H, 4.77; N, 9.39.

E-isomer: di-maleate salt, mp 154-155° C. ¹H NMR (300 MHz, DMSO-d₆) δ 2.90 (m, 4H), 3.49 (m, 4H), 3.86 (m, 5H), 6.20 (s, 4H), 6.70 (d-d, J=7 Hz and 4 Hz, 1H), 6.87 (d, J=9 Hz, 1H), 7.60 (m, 5H), 8.13 (m, 1H); MS (DCI/NH₃) m/z 345 (M+H)⁺. Anal. calcd for C₂₆H₂₉ClN₄O₉: C, 54.12; H, 5.07; N, 9.71. Found: C, 54.39; H, 4.89; N, 9.61.

### EXAMPLE 5

### (1E)-1-(3,4-dimethylphenyl)-3-(4-pyridin-2-ylpiperazin-1-yl)propan-1-one O-methyloxime and

### (1Z)-1-(3,4-dimethylphenyl)-3-(4-pyridin-2-ylpiperazin-1-yl)propan-1-one O-methyloxime

### EXAMPLE 5A

### 1-(3,4-dimethylphenyl)-3-(4-pyridin-2-ylpiperazin-1-yl)propan-1-one

The title compound was prepared using the procedure described in Example 1A except using 3,4-dimethylacetophenone instead of 3-chloroacetophenone. ¹H NMR (300 MHz, DMSO-d₆) δ 2.28 (s, 6H), 2.44 (t, J=4 Hz, 4H), 2.70 (t, J=7 Hz, 2H), 3.20 (t, J=7 Hz, 2H), 3.44 (t, J=4 Hz, 4H), 6.62 (d-d, J=7 Hz, 4 Hz, 1H), 6.80 (d, J=9 Hz, 1H), 7.28 (d, J=9 Hz, 1H), 7.51 (m, 1H), 7.73 (m, 1H), 7.79 (m, 1H), 8.10 (m, 1H); MS (DCI/NH₃) m/z 324 (M+H)⁺.

### EXAMPLE 5B

### (1E)-1-(3,4-dimethylphenyl)-3-(4-pyridin-2-ylpiperazin-1-yl)propan-1-one O-methyloxime and

### (1Z)-1-(3,4-dimethylphenyl)-3-(4-pyridin-2-ylpiperazin-1-yl)propan-1-one O-methyloxime

The title compounds were prepared using the procedure described in Example 1B except using the product from Example 5A instead of the product from Example 1A.

E-isomer: maleate salt, mp 166-167° C. ¹H NMR (300 MHz, DMSO-d₆) δ 2.23 and 2.26 (2s, 6H), 3.20 (m, 12H), 3.97 (s, 3H), 6.06 (s, 2H), 6.74 (d-d, J=7 Hz and 4 Hz, 1H), 6.95 (d, J=9 Hz, 1H), 7.20 (d, J=9 Hz, 1H), 7.40 (m, 1H), 7.46 (m, 1H), 7.60 (m, 1H), 8.16 (m, 1H); MS (DCI/NH₃) m/z 353 (M+H)⁺. Anal. calcd for C₂₅H₃₂N₄O₅.0.6H2O: C, 62.64; H, 6.98; N, 11.69. Found: C, 62.46; H, 6.63; N, 11.58.

Z-isomer: maleate salt, mp 130-131° C. ¹H NMR (300 MHz, DMSO-d₆) δ 2.23 (s, 6H), 3.12 (m, 12H), 3.76 (s, 3H), 6.06 (s, 2H), 6.74 (d-d, J=7 Hz and 4 Hz, 1H), 6.95 (d, J=9 Hz, 1H), 7.24 (m,3H), 7.60 (m, 1H), 8.16 (m, 1H); MS (DCI/NH₃) m/z 353 (M+H)⁺. Anal. calcd for C₂₅H₃₂N₄O₅: C, 64.09; H, 6.88; N, 11.96. Found: C, 64.00; H, 6.86; N, 11.67.

### EXAMPLE 6

### (1E)-1-(3-chloro-4-fluorophenyl)-3-(4-pyridin-2-ylpiperazin-1-yl)propan-1-one O-methyloxime and

### (1Z)-1-(3-chloro-4-fluorophenyl)-3-(4-pyridin-2-ylpiperazin-1-yl)propan-1-one O-methyloxime

### EXAMPLE 6A

### 1-(3-chloro-4-fluorophenyl)-3-(4-pyridin-2-ylpiperazin-1-yl)propan-1-one

The title compound was prepared using the procedure described in Example 1A except using 3-chloro-4-fluoroacetophenone for 3-chloroacetophenone, to provide the title compound. ¹H NMR (300 MHz, DMSO-d₆) δ 2.44 (t, J=4 Hz, 4H), 2.70 (t, J=7 Hz, 2H), 3.30 (t, J=7 Hz, 2H), 3.44 (t, J=4 Hz, 4H), 6.62 (d-d, J=7 Hz, 4 Hz, 1H), 6.80 (d, J=9 Hz, 1H), 7.52 (m, 2H), 8.02 (m, 1H), 8.10 (m, 1H), 8.22 (d-d, J=7 Hz, 3 Hz, 1H); MS (DCI/NH₃) m/z 348 (M+H)⁺.

### EXAMPLE 6B

### (1E)-1-(3-chloro-4-fluorophenyl)-3-(4-pyridin-2-ylpiperazin-1-yl)propan-1-one O-methyloxime and

### (1Z)-1-(3-chloro-4-fluorophenyl)-3-(4-pyridin-2-ylpiperazin-1-yl)propan-1-one O-methyloxime

The title compounds were prepared using the procedure described in Example 1B except using the product from Example 6A instead of the product from Example 1A.

E-isomer: maleate salt, mp 161-162° C. ¹H NMR (300 MHz, DMSO-d₆) δ 3.18 (m, 12H), 3.97 (s, 3H), 6.06 (s, 2H), 6.74 (d-d, J=7 Hz and 4 Hz, 1H), 6.94 (d, J=9 Hz, 1H), 7.52 (t, J=9 Hz, 1H), 7.60 (m, 1H), 7.71 (m, 1H), 7.87 (d-d, J=7 Hz and 3 Hz, 1H), 8.16 (m, 1H); MS (DCI/NH₃) m/z 377 (M+H)⁺. Anal. calcd for C₂₃H₂₆FClN₄O₅: C, 56.04; H, 5.32; N, 11.37. Found: C, 55.95; H, 5.15; N, 11.12.

Z-isomer: maleate salt, mp 143-144° C. ¹H NMR (300 MHz, DMSO-d₆) δ 3.22 (m, 12H), 3.80 (s, 3H), 6.06 (s, 2H), 6.73 (d-d, J=7 Hz and 4 Hz, 1H), 6.94 (d, J=9 Hz, 1H), 7.58 (m,3H), 7.77 (m, 1H), 8.16 (m, 1H); MS (DCI/NH₃) m/z 377 (M+H)⁺.

Anal. calcd for C₂₃H₂₆FClN₄O₅.0.2H₂O: C, 55.64; H, 5.36; N, 11.28. Found: C, 55.58; H, 5.09; N, 10.95.

### EXAMPLE 7

### (1E)-1-(3-methylphenyl)-3-(4-pyridin-2-ylpiperazin-1-yl)propan-1-one O-methyloxime and

### (1Z)-1-(3-methylphenyl)-3-(4-pyridin-2-ylpiperazin-1-yl)propan-1-one O-methyloxime

### EXAMPLE 7A

### 1-(3-methylphenyl)-3-(4-pyridin-2-ylpiperazin-1-yl)propan-1-one

The title compound was prepared using the procedure from Example 1A except using 3-methylacetophenone instead of 3-chloroacetophenone. MS (DCI/NH₃) m/z 310 (M+H)⁺.

### EXAMPLE 7B

### (1E)-1-(3-methylphenyl)-3-(4-pyridin-2-ylpiperazin-1-yl)propan-1-one O-methyloxime and

### (1Z)-1-(3-methylphenyl)-3-(4-pyridin-2-ylpiperazin-1-yl)propan-1-one O-methyloxime

The title compounds were prepared using the procedure described in Example 1B except using the product from Example 7A instead of the product from Example 1A.

E-isomer: maleate salt, mp 124-125° C. ¹H NMR (300 MHz, DMSO-d₆) δ 2.30 (s, 3H), 3.25 (m, 12H), 3.90 (s, 3H), 6.08 (s, 2H), 6.72 (d-d, J=7 Hz and 4 Hz, 1H), 6.91 (d, J=9 Hz, 1H), 7.28 (m, 4H), 7.60 (m, 1H), 8.16 (m, 1H); MS (DCI/NH₃) m/z 339 (M+H)⁺. Anal. calcd for C₂₄H₃₀N₄O₅.0.4H₂O: C, 62.43; H, 6.72; N, 12.13. Found: C, 62.78; H, 6.75; N, 11.70.

Z-isomer: maleate salt, mp 119-121° C. ¹H NMR (300 MHz, DMSO-d₆) δ 2.18 (s, 3H), 2.87 (m, 2H), 3.30 (m, 12H), 3.74 (s, 3H), 6.08 (s, 2H), 6.74 (d-d, J=7 Hz and 4 Hz, 1H), 6.94 (d, J=9 Hz, 1H), 7.14 (m, 1H), 7.25 (m, 4H), 7.60 (m, 1H), 8.16 (m, 1H); MS (DCI/NH₃) m/z 339 (M+H)⁺. Anal. calcd for C₂₄H₃₀N₄O₅.0.5H₂O: C, 62.19; H, 6.74; N, 12.09. Found: C, 62.58; H, 6.62; N, 11.63.

### EXAMPLE 9

### (1E)-1-(4-fluorophenyl)-3-(4-pyridin-2-ylpiperazin-1-yl)propan-1-one O-methyloxime and

### (1Z)-1-(4-fluorophenyl)-3-(4-pyridin-2-ylpiperazin-1-yl)propan-1-one O-methyloxime

### EXAMPLE 9A

### 1-(4-fluorophenyl)-3-(4-pyridin-2-ylpiperazin-1-yl)propan-1-one

The title compound was prepared using the procedure described in Example 1A except using 4-fluoroacetophenone for 3-chloroacetophenone. MS (DCI/NH₃) m/z 314 (M+H)⁺.

### EXAMPLE 9B

### (1E)-1-(4-fluorophenyl)-3-(4-pyridin-2-ylpiperazin-1-yl)propan-1-one O-methyloxime and

### (1Z)-1-(4-fluorophenyl)-3-(4-pyridin-2-ylpiperazin-1-yl)propan-1-one O-methyloxime

The title compounds were prepared using the procedure described in Example 1B except using the product from Example 9A instead of the product from Example 1A.

E-isomer: maleate salt, mp 157-159° C. ¹H NMR (300 MHz, DMSO-d₆) δ 3.20 (m, 12H), 3.98 (s, 3H), 6.08 (s, 2H), 6.73 (d-d, J=7 Hz, 4 Hz, 1H), 6.95(d, J=9 Hz, 1H), 7.30 (m, 1H), 7.55 (m, 4H), 8.16 (m, 1H); MS (DCI/NH₃) m/z 343 (M+H)⁺. Anal. calcd for C₂₃H₂₇FN₄O₅: C, 60.25; H, 5.94; N, 12.22. Found: C, 60.40; H, 6.05; N, 12.23.

Z-isomer: maleate salt, mp 122-124° C. ¹H NMR (300 MHz, DMSO-d₆) δ 3.30 (m, 12H), 3.80 (s, 3H), 6.08 (s, 2.5H), 6.75 (d-d, J=7 Hz, 4 Hz, 1H), 6.95 (d, J=9 Hz, 1H), 7.34 (m, 3H), 7.50 (m, 1H), 7.61(m, 1H), 8.16 (m, 1H); MS (DCI/NH₃) m/z 343 (M+H)⁺. Anal. calcd for C₂₃H₂₇FN₄O₅: C, 60.25; H, 5.94; N, 12.22. Found: C, 60.14; H, 5.98; N, 11.88.

### EXAMPLE 10

### (1E)-1-(3,4-dichlorophenyl)-2-(4-pyridin-2-ylpiperazin-1-yl)ethanone O-methyloxime and

### (1Z)-1-(3,4-dichlorophenyl)-2-(4-pyridin-2-ylpiperazin-1-yl)ethanone O-methyloxime

### EXAMPLE 10A

### 1-(3,4-dichlorophenyl)-2-(4-pyridin-2-ylpiperazin-1-yl)ethanone

The title compound was prepared using the procedure described in Example 3A except using 3,4-dichlorophenacyl bromide instead of 4-fluorophenacyl chloride. ¹H NMR (300 MHz, DMSO-d₆) δ 2.60 (t, J=4 Hz, 4H), 3.47 (t, J=4 Hz, 4H), 3.91 (s, 2H), 6.62 (d-d, J=7 Hz, 4 Hz, 1H), 6.81(d, J=9 Hz, 1H), 7.31 (m, 1H), 7.82 (d, J=9 Hz, 1H), 7.97 (d-d, J=9 Hz, 3 Hz, 1H), 8.10 (d-d, J=6 Hz, 3 Hz, 1H), 8.21 (d, J=3 Hz,1H); MS (DCI/NH₃) m/z 350 (M+H)⁺.

### EXAMPLE 10B

### (1E)-1-(3,4-dichlorophenyl)-2-(4-pyridin-2-ylpiperazin-1-yl)ethanone O-methyloxime and

### (1Z)-1-(3,4-dichlorophenyl)-2-(4-pyridin-2-ylpiperazin-1-yl)ethanone O-methyloxime

The title compounds were prepared using the procedure described in Example 1B except using the product from Example 10A instead of the product from Example 1A.

Z-isomer: di-maleate salt, mp 133-135° C. ¹H NMR (300 MHz, DMSO-d₆) δ 2.75 (m, 4H), 3.51 (m, 4H), 3.98 (m+s, 5H), 6.22 (s, 4H), 6.68 (d-d, J=7 Hz and 4 Hz, 1H), 6.85 (d, J=9 Hz, 1H), 7.57 (m, 1H), 7.70 (d, J=9 Hz, 1H), 7.77 (d-d, J=9 Hz, 3 Hz, 1H), 8.02 (d, J=3 Hz, 1H), 8.10 (m, 1H); MS (DCI/NH₃) m/z 379 (M+H)⁺. Anal. calcd for C₂₆H₂₈Cl₂N₄O₉: C, 51.07; H, 4.62; N, 9.16. Found: C, 51.16; H, 4.54; N, 8.90.

E-isomer: di-maleate salt, mp 146-148° C. ¹H NMR (300 MHz, DMSO-d₆) δ 2.90 (m, 4H), 3.55 (m, 4H), 3.86 (m+s, 5H), 6.20 (s, 4H), 6.70 (d-d, J=7 Hz and 4 Hz, 1H), 6.87 (d, J=9 Hz, 1H), 7.60 (m, 2H), 7.75 (d, J=9 Hz, 1H), 7.88 (d, J=3 Hz, 1H), 8.13 (m, 1H); MS (DCI/NH₃) m/z 379 (M+H)⁺. Anal. calcd for C₂₆H₂₈Cl₂N₄O₉: C, 51.07; H, 4.62; N, 9.16. Found: C, 51.11; H, 4.64; N, 9.04.

### EXAMPLE 11

### (1E)-1-(2-methylphenyl)-3-(4-pyridin-2-ylpiperazin-1-yl)propan-1-one O-ethyloxime and

### (1Z)-1-(2-methylphenyl)-3-(4-pyridin-2-ylpiperazin-1-yl)propan-1-one O-ethyloxime

### EXAMPLE 11A

### 1-(2-methylphenyl)-3-(4-pyridin-2-ylpiperazin-1-yl)propan-1-one

The title compound was prepared using the procedure described in Example 1A except using 2-methylacetophenone instead of 3-chloroacetophenone. ¹H NMR (300 MHz, DMSO-d₆) δ 2.40 (s, 3H), 2.44 (t, J=4 Hz, 4H), 2.65 (t, J=7 Hz, 2H), 3.11 (t, J=7 Hz, 2H), 3.40 (t, J=4 Hz, 4H), 6.62 (d-d, J=7 Hz, 4 Hz, 1H), 6.80 (d, J=9 Hz, 1H), 7.30 (m, 2H), 7.41 (m, 1H), 7.50 (m, 1H), 7.75 (m, 1H), 8.10 (m, 1H); MS (DCI/NH₃) m/z 310 (M+H)⁺.

### EXAMPLE 11B

### (1E)-1-(2-methylphenyl)-3-(4-pyridin-2-ylpiperazin-1-yl)propan-1-one O-ethyloxime and

### (1Z)-1-(2-methylphenyl)-3-(4-pyridin-2-ylpiperazin-1-yl)propan-1-one O-ethyloxime

The product from Example 11A (309 mg, 1 mmol) and O-ethylhydroxylamine hydrochloride (290 mg, 3 mmol) were combined in pyridine (15 mL) and stirred for 18 hours at ambient temperature. The reaction mixture was concentrated under reduced pressure, treated with saturated NaHCO₃, and extracted with ethyl acetate. The organic layer was washed with water, brine, dried over anhydrous MgSO₄, filtered, and the filtrate was concentrated under reduced pressure. The residue was purified by column chromatography (silica gel, CH₂Cl₂:acetone 4:1) to provide the title compounds.

E-isomer: maleate salt, mp 127-129° C. ¹H NMR (300 MHz, DMSO-d₆) δ 1.27 (t, J=7 Hz, 3H), 2.32 (s, 3H), 3.10 (m, 12H), 4.15 (q, J=7 Hz, 2H), 6.08 (s, 1.2H), 6.73 (d-d, J=7 Hz, 4 Hz, 1H), 6.92(d, J=9 Hz, 1H), 7.47 (m, 4H), 7.60 (m, 1H), 8.14 (m, 1H); MS (DCI/NH₃) m/z 353 (M+H)⁺. Anal. calcd for C₂₁H₂₈N₄O.1.2C₄H₄O₄: C, 63.01; H, 6.72; N, 11.39. Found: C, 63.02; H, 6.74; N, 11.21.

Z-isomer: maleate salt, mp 118-120° C. ¹H NMR (300 MHz, DMSO-d₆) δ 1.27 (t, J=7 Hz, 3H), 2.18 (s, 3H), 2.96)t, J=7 Hz, 2H), 3.30 (m, 10H), 4.00 (q, J=7 Hz, 2H), 6.08 (s, 1.2H), 6.73 (d-d, J=7 Hz, 4 Hz, 1H), 6.94 (d, J=9 Hz, 1H), 7.15 (m, 1H), 7.25 (m, 3H), 7.60 (m, 1H), 8.15 (m, 1H); MS (DCI/NH₃) m/z 353 (M+H)⁺. Anal. calcd for C₂₁H₂₈N₄O.1.2C₄H₄O₄: C, 63.01; H, 6.72; N, 11.39. Found: C, 63.35; H, 6.76; N, 11.21.

### EXAMPLE 12

### (1E)-1-(2-methylphenyl)-3-(4-pyridin-2-ylpiperazin-1-yl)propan-1-one O-methyloxime and

### (1Z)-1-(2-methylphenyl)-3-(4-pyridin-2-ylpiperazin-1-yl)propan-1-one O-methyloxime

The title compounds were prepared using the procedure described in Example 1B except using the product from Example 11A instead of the product from Example 1A.

E-isomer: ¹H NMR (300 MHz, DMSO-d₆) δ 2.34 (s, 3H), 2.46 (m, 4H), 2.91 (m, 2H), 3.30 (m, 2H), 3.42 (m, 4H), 3.90 (s, 3H), 6.62 (d-d, J=7 Hz, 4 Hz, 1H), 6.80 (d, J=9 Hz, 1H), 7.21 (m, 1H), 7.30 (t, J=7 Hz, 1H), 7.50 (m, 3H), 8.10 (m, 1H); MS (DCI/NH₃) m/z 339 (M+H)⁺. Anal. calcd for C₂₀H₂₆N₄O: C,; H, 6.; N,. Found: C,; H,; N,.

Z-isomer: ¹H NMR (300 MHz, DMSO-d₆) δ 2.31 (s, 3H), 2.40 (m, 6H), 2.68 (t, J=7 Hz, 2H), 3.21 (m, 4H), 3.70 (s, 3H), 6.62 (d-d, J=7 Hz, 4 Hz, 1H), 6.80 (d, J=9 Hz, 1H), 7.18 (m, 3H), 7.28 (m, 1H), 7.50 (m, 1H), 8.10 (m, 1H); MS (DCI/NH₃) m/z 339 (M+H)⁺. Anal. calcd for C₂₀H₂₆N₄O: C,; H,; N,. Found: C,; H,; N,.

### EXAMPLE 13

### 3-[(1E)-N-methoxy-3-(4-pyridin-2-ylpiperazin-1-yl)propanimidoyl]benzonitrile and

### 3-[(1Z)-N-methoxy-3-(4-pyridin-2-ylpiperazin-1-yl)propanimidoyl]benzonitrile

### EXAMPLE 13A

### 3-[3-(4-pyridin-2-ylpiperazin-1-yl)propanoyl]benzonitrile

The title compound was prepared using the procedure described in Example 1A except using 3-acetylbenzonitrile instead of 3-chloroacetophenone. MS (DCI/NH₃) m/z 321 (M+H)⁺.

### EXAMPLE 13B

### 3-[(1E)-N-methoxy-3-(4-pyridin-2-ylpiperazin-1-yl)propanimidoyl]benzonitrile and

### 3-[(1Z)-N-methoxy-3-(4-pyridin-2-ylpiperazin-1-yl)propanimidoyl]benzonitrile

The title compounds were prepared using the procedure described in Example 1B except using the product from Example 13A instead of the product from Example 1A.

E-isomer: maleate salt, mp 161-163° C. ¹H NMR (300 MHz, DMSO-d₆) δ 3.20 (m, 12H), 4.00 (s, 3H), 6.08 (s, 2.8H), 6.74 (d-d, J=7 Hz, 4 Hz, 1H), 6.96 (d, J=9 Hz, 1H), 7.64 (m, 2H), 7.93 (m, 1H), 8.03 (m, 1H), 8.10 (m, 1H), 8.16 (m, 1H); MS (DCI/NH₃) m/z 350 (M+H)⁺. Anal. calcd for C₂₀H₂₃N₅O.1.4C₄H₄O₄: C, 60.06; H, 5.63; N, 13.68. Found: C, 59.71; H, 5.65; N, 13.27.

Z-isomer: white solid, mp 105-108° C. ¹H NMR (300 MHz, DMSO-d₆) δ 2.38 (m, 6H), 2.73 (t, J=7 Hz, 2H), 3.40 (m, 4H), 3.73 (s, 3H), 6.62 (d-d, J=7 Hz, 4 Hz, 1H), 6.80 (d, J=9 Hz, 1H), 7.50 (m, 1H), 7.62 (t, J=9 Hz, 1H), 7.75 (m, 1H), 7.83 (m, 1H), 7.89 (m, 1H), 8.09 (m, 1H); MS (DCI/NH₃) m/z 350 (M+H)⁺. Anal. calcd for C₂₀H₂₃N₅O: C, 68.74; H, 6.63; N, 20.04. Found: C, 68.53; H, 6.64; N, 19.91.

### EXAMPLE 14

### (1E)-1-(2,4-dichlorophenyl)-2-(4-pyridin-2-ylpiperazin-1-yl)ethanone O-methyloxime and

### (1Z)-1-(2,4-dichlorophenyl)-2-(4-pyridin-2-ylpiperazin-1-yl)ethanone O-methyloxime

### EXAMPLE 14A

### 1-(2,4-dichlorophenyl)-2-(4-pyridin-2-ylpiperazin-1-yl)ethanone

The title compound was prepared using the procedure described in Example 3A except using 2,4-dichlorophenacyl bromide instead of 4-fluorophenacyl chloride. MS (DCI/NH₃) m/z 350 (M+H)⁺.

### EXAMPLE 14B

### (1E)-1-(2,4-dichlorophenyl)-2-(4-pyridin-2-ylpiperazin-1-yl)ethanone O-methyloxime and

### (1Z)-1-(2,4-dichlorophenyl)-2-(4-pyridin-2-ylpiperazin-1-yl)ethanone O-methyloxime

The title compounds were prepared using the procedure described in Example 1B except using the product from Example 14A instead of the product from Example 1A.

E-isomer: di-tartrate salt, ¹H NMR (300 MHz, DMSO-d₆) δ 2.21 (m, 4H), 3.23 (m, 6H), 3.90 (s, 3H), 4.30 (s, 4.4H), 6.60 (d-d, J=7 Hz and 4 Hz, 1H), 6.75 (d, J=9 Hz, 1H), 7.48 (m, 3H), 7.66 (d, J=3 Hz, 1H), 8.05 (m, 1H); MS (DCI/NH₃) m/z 379 (M+H)⁺. Anal. calcd for C₁₈H₂₀Cl₂N₄O.2.2C₄H₆O₆.0.6H₂O: C, 44.69; H, 4.81; N, 7.78. Found: C, 44.90; H, 4.95; N, 7.14.

Z-isomer: di-tartrate salt, ¹H NMR (300 MHz, DMSO-d₆) δ 2.54 (m, 4H), 3.40 (m, 6H), 3.78 (s, 3H), 4.30 (s, 4.4H), 6.62 (d-d, J=7 Hz and 4 Hz, 1H), 6.77 (d, J=9 Hz, 1H), 7.35 (d, J=9 Hz, 1H), 7.50 (m, 2H), 7.66 (d, J=3 Hz, 1H), 8.08 (m, 1H); MS (DCI/NH₃) m/z 379 (M+H)⁺. Anal. calcd for C₁₈H₂₀Cl₂N₄O.2.2C₄H₆O₆.0.1H₂O: C, 45.26; H, 4.73; N, 7.88. Found: C, 45.63; H, 5.02; N, 7.40.

### EXAMPLE 16

### (1E)-1-phenyl-3-(4-pyridin-2-ylpiperazin-1-yl)propan-1-one oxime and

### (1Z)-1-phenyl-3-(4-pyridin-2-ylpiperazin-1-yl)propan-1-one oxime

### EXAMPLE 16A

### 1-phenyl-3-(4-pyridin-2-ylpiperazin-1-yl)propan-1-one

1-(2-Pyridinyl)piperazine (0.77 mL, 5 mmol), 3′-chloro-propiophenone (840 mg, 5 mmol), and K₂CO₃ (690 mg, 0.5 mmol)) were combined in DMF (25 mL) and heated at 40° C. for 14 hours. The mixture was poured into water and extracted with ethyl acetate. The organic layer was combined, washed with water, brine, dried over anhydrous MgSO₄, filtered, and the filtrate was concentrated under reduced pressure. The residue was purified by column chromatography (hexanes:ethyl acetate 1:1) to provide the title compound. ¹H NMR (300 MHz, DMSO-d₆) δ 2.50 (m, 4H), 2.72 (t, J=7 Hz, 2H), 3.25 (t, J=7 Hz, 2H), 3.42 (t, J=4.5 Hz, 4H), 6.61 (d-d, J=7 Hz, 4 Hz, 1H), 6.80 (d, J=9 Hz, 1H), 7.52 (m, 3H), 7.63 (m, 1H), 8.00 (m, 2H), 8.10 (m, 1H); MS (DCI/NH₃) m/z 296 (M+H)⁺.

### EXAMPLE 16B

### (1E)-1-phenyl-3-(4-pyridin-2-ylpiperazin-1-yl)propan-1-one oxime and

### (1Z)-1-phenyl-3-(4-pyridin-2-ylpiperazin-1-yl)propan-1-one oxime

The title compounds were prepared using the procedure described in Example 15B except using the product from Example 16A instead of the product from Example 15A.

E-isomer: mp 169-170° C. ¹H NMR (300 MHz, DMSO-d₆) δ 2.50 (m, 6H), 2.95 (t, J=7 Hz, 2H), 3.42 (t, J=4.5 Hz, 4H), 6.61 (d-d, J=7 Hz, 4 Hz, 1H), 6.80 (d, J=9 Hz, 1H), 7.22 (t, J=9 Hz, 2H), 7.39 (m, 3H), 7.51 (m, 1H), 7.65 (m, 2H), 8.10 (m, 1H), 11.13 (s, 1H); MS (DCI/NH₃) m/z 311 (M+H)⁺. Anal. calcd for C₁₈H₂₂N₄O.0.25H₂O: C, 68.66; H, 7.04; N, 17.79. Found: C, 68.88; H, 6.95; N, 17.99.

Z-isomer: mp 130-132° C. ¹H NMR (300 MHz, DMSO-d₆) δ 2.40 (m, 4H), 2.70 (t, J=7 Hz, 2H), 3.35 (m, 2H), 3.42 (m, 4H), 6.61 (d-d, J=7 Hz, 4 Hz, 1H), 6.79 (d, J=9 Hz, 1H), 7.40 (m, 6H), 8.09 (m, 1H), 10.58 (s, 1H); MS (DCI/NH₃) m/z 311 (M+H)⁺.

### EXAMPLE 17

### 1,5-diphenyl-2-[(4-pyridin-2-ylpiperazin-1-yl)methyl]pentane-1,5-dione dioxime

### EXAMPLE 17A

### 1,5-diphenyl-2-[(4-pyridin-2-ylpiperazin-1-yl)methyl]pentane-1,5-dione

The title compound was isolated as a side product from the experiment described in Example 16A.

### EXAMPLE 17B

### 1,5-diphenyl-2-[(4-pyridin-2-ylpiperazin-1-yl)methyl]pentane-1,5-dione dioxime

The title compound was prepared using the procedure described in Example 15B except using the product from Example 17A instead of the product from Example 15A.

E,Z-isomer: mp 75-78° C. ¹H NMR (300 MHz, DMSO-d₆) δ 1.60 (m, 2H), 2.37 (m, 6H), 2.80 (t, J=7 Hz, 2H), 2.93 (m, 1H), 3.40 (m, 4H), 6.62 (d-d, J=7 Hz, 4 Hz, 1H), 6.80 (d, J=9 Hz, 1H), 7.35 (m, 8H), 7.50 (m, 1H), 7.63 (m, 2H), 8.10 (m, 1H), 10.55 (s, 1H), 11.13 (s, 1H); MS (DCI/NH₃) m/z 458 (M+H)⁺.

E,E-isomer: mp 181-184° C. ¹H NMR (300 MHz, DMSO-d₆) δ 1.80 (m, 2H), 2.45 (m, 3H), 2.60 (m, 3H), 2.80 (m, 1H), 3.39 (m, 4H), 6.61 (d-d, J=7 Hz, 4 Hz, 1H), 6.79 (d, J=9 Hz, 1H), 7.35 (m, 5H), 7.50 (m, 5H), 7.62 (m, 1H), 8.09 (m, 1H), 11.13 (s, 1H), 11.16 (s, 1H); MS (DCI/NH₃) m/z 458 (M+H)⁺. Anal. calcd for C₂₇H₃₁N₅O₂.2H₂O: C, 65.70; H, 6.33; N, 14.19. Found: C, 65.90; H, 6.38; N, 13.80.

### EXAMPLE 18

### (1E)-1-phenyl-3-(4-pyrimidin-2-ylpiperazin-1-yl)propan-1-one oxime and

### (1Z)-1-phenyl-3-(4-pyrimidin-2-ylpiperazin-1-yl)propan-1-one oxime

### EXAMPLE 18A

### 1-phenyl-3-(4-pyrimidin-2-ylpiperazin-1-yl)propan-1-one

The title compound was prepared using the procedure described in Example 16A except using 1-(2-pyrimidinyl)piperazine instead of 1-(2-pyridinyl)piperazine. MS (DCI/NH₃) m/z 297 (M+H)⁺.

### EXAMPLE 18B

### (1E)-1-phenyl-3-(4-pyrimidin-2-ylpiperazin-1-yl)propan-1-one oxime and

### (1Z)-1-phenyl-3-(4-pyrimidin-2-ylpiperazin-1-yl)propan-1-one oxime

The title compound was prepared using the procedure described in Example 15B except using the product from Example 18A instead of the product from Example 15A.

E-isomer: mp 175-177° C. ¹H NMR (300 MHz, DMSO-d₆) δ 2.50 (m, 6H), 2.95 (m, 2H), 3.70 (t, J=4.5 Hz, 4H), 6.61 (t, J=4.5 Hz, 1H), 7.39 (m, 3H), 7.64 (m, 2H), 8.33 (d, J=4.5 Hz, 1H), 11.23 (s, 1H); MS (DCI/NH₃) m/z 312 (M+H)⁺. Anal. calcd for C₁₇H₂₁N₅O.0.15H₂O: C, 65.01; H, 6.74; N, 22.30. Found: C, 65.29; H, 6.93; N, 21.79.

Z-isomer: ¹H NMR (300 MHz, DMSO-d₆) δ 2.40 (m, 6H), 2.69 (t, J=7 Hz, 2H), 3.67 (t, J=4.5 Hz, 4H), 6.60 (t, J=4.5 Hz, 1H), 7.40 (m, 5H), 8.35 (d, J=4.5 Hz, 1H), 10.58 (s, 1H); MS (DCI/NH₃) m/z 312 (M+H)⁺.

### EXAMPLE 19

### 1,5-diphenyl-2-[(4-pyrimidin-2-ylpiperazin-1-yl)methyl]pentane-1,5-dione dioxime

### EXAMPLE 19A

### 1,5-diphenyl-2-[(4-pyrimidin-2-ylpiperazin-1-yl)methyl]pentane-1,5-dione

The title compound was isolated as a side product from the experiment described in Example 18A.

### EXAMPLE 19B

### 1,5-diphenyl-2-[(4-pyrimidin-2-ylpiperazin-1-yl)methyl]pentane-1,5-dione dioxime

The title compound was prepared using the procedure described in Example 15B except using the product from Example 19A instead of the product from Example 15A.

E,Z-isomer: mp 75-78° C. ¹H NMR (300 MHz, DMSO-d₆) δ 1.60 (m, 2H), 2.37 (m, 6H), 2.80 (t, J=7 Hz, 2H), 2.93 (m, 1H), 3.65 (m, 4H), 6.60 (t, J=4.5 Hz, 1H), 7.26 (m, 5H), 7.63 (m, 2H), 8.33 (d, J=4.5 Hz, 1H), 10.55 (s, 1H), 11.17 (s, 1H); MS (DCI/NH₃) m/z 459 (M+H)⁺.

E,E-isomer: mp 181-184° C. ¹H NMR (300 MHz, DMSO-d₆) δ 1.80 (m, 2H), 2.45 (m, 3H), 2.60 (m, 3H), 2.59 (m, 2H), 2.80 (m, 1H), 3.62 (m, 4H), 6.60 (t, J=4.5 Hz, 1H), 7.32 (m, 3H), 7.38 (m, 3H), 7.50 (m, 4H), 8.32 (m, 1H), 11.12 (s, 1H), 11.16 (s, 1H); MS (DCI/NH₃) m/z 459 (M+H)⁺.

### EXAMPLE 21

### 1-(4-fluorophenyl)-3-(4-pyridin-2-ylpiperazin-1-yl)propan-1-one oxime

### EXAMPLE 21A

### 1-(4-fluorophenyl)-3-(4-pyridin-2-ylpiperazin-1-yl)propan-1-one

The title compound was prepared using the procedure described in Example 16A except using 3′-chloro-4-fluoro-propiophenone instead of 3′-chloro-propiophenone. MS (DCI/NH₃) m/z 314(M+H)⁺.

### EXAMPLE 21B

### 1-(4-fluorophenyl)-3-(4-pyridin-2-ylpiperazin-1-yl)propan-1-one oxime

The title compound was prepared using the procedure described in Example 15B except using the product from Example 21A instead of the product from Example 15A.

E-isomer: mp 159-160° C. ¹H NMR (300 MHz, DMSO-d₆) δ 2.50 (m, 6H), 2.93 (m, 2H), 3.43 (t, J=4.5 Hz, 4H), 6.61 (dd, J=6 Hz, 9 Hz, 1H), 7.80 (d, J=9 Hz, 1H), 7.22 (t, J=9 Hz, 2H), 7.51 (m, 1H), 7.70 (m, 2H), 8.10 (m, 1H), 11.26 (s, 1H); MS (DCI/NH₃) m/z 329 (M+H)⁺. Anal. calcd for C₁₈H₂₁FN₄O.0.5H₂O: C, 64.08; H, 6.57; N, 16.61. Found: C, 64.17; H, 6.32; N, 16.63.

### EXAMPLE 22

### (1E)-1-(4-chlorophenyl)-3-(4-pyridin-2-ylpiperazin-1-yl)propan-1-one oxime

The title compound was prepared using the procedure described in Example 15B except using the product from Example 2A instead of the product from Example 15A.

E-isomer: mp 188-190° C. ¹H NMR (300 MHz, DMSO-d₆) δ 2.50 (m, 6H), 2.93 (m, 2H), 3.43 (t, J=4.5 Hz, 4H), 6.61 (dd, J=4 Hz, 7 Hz, 1H), 6.80 (d, J=9 Hz, 1H), 7.50 (m, 3H), 7.66 (m, 2H), 8.10 (m, 1H), 11.38 (s, 1H); MS (DCI/NH₃) m/z 345 (M+H)⁺.

Anal. calcd for C₁₈H₂₁ClN₄O: C, 62.69; H, 6.14; N, 16.25. Found: C, 62.49; H, 6.04; N, 16.04.

### EXAMPLE 23

### (1E)-1-phenyl-3-(4-pyridin-2-ylpiperazin-1-yl)propan-1-one O-ethyloxime and

### (1Z)-1-phenyl-3-(4-pyridin-2-ylpiperazin-1-yl)propan-1-one O-ethyloxime

The mixture of E-and Z-isomers from Example 16B (248 mg, 0.8 mmol) in tert-butanol (25 mL) was treated with powdered potassium t-butoxide (90 mg, 0.8 mmol) and refluxed for ˜30 minutes. The mixture was allowed to cool to room temperature and was treated with ethyl iodide (0.065 mL, 0.8 mmol) and then refluxed for 1 hour. The mixture was concentrated under reduced pressure and the residue was partitioned between water and ethyl acetate. The organic layer was washed with brine, dried over anhydrous MgSO₄, filtered, and the filtrate was concentrated under reduced pressure. The residue was purified by column chromatography to provide the title compounds.

E-isomer: maleate salt, mp 150-151° C. ¹H NMR (300 MHz, DMSO-d₆) δ 1.28 (t, J=7 Hz, 3H), 3.25 (m, 12H), 4.21 (t, J=7 Hz, 2H), 6.07 (s, 2H), 6.73 (dd, J=4 Hz, 7 Hz, 1H), 6.95 (d, J=9 Hz, 1H), 7.44 (m, 3H), 7.60 (m, 1H), 7.70 (m, 2H), 8.16 (m, 1H); MS (DCI/NH₃) m/z 339 (M+H)⁺. Anal. calcd for C₂₄H₃₀N₄O₅: C, 63.42; H, 6.65; N, 12.33. Found: C, 63.05; H, 6.56; N, 12.07.

Z-isomer: ¹H NMR (300 MHz, DMSO-d₆) δ 1.13 (t, J=7 Hz, 3H), 2.41 (m, 6H), 2.70 (t, J=7 Hz, 2H), 3.42 (t, J=4.5 Hz, 4H), 3.99 (t, J=7 Hz, 2H), 6.61 (dd, J=4 Hz, 7 Hz, 1H), 6.80 (d, J=9 Hz, 1H), 7.40 (m, 5H), 7.70 (m, 1H), 8.09 (m, 1H); MS (DCI/NH₃) m/z 339 (M+H)⁺.

### EXAMPLE 24

### (1E)-1-phenyl-3-(4-pyridin-2-ylpiperazin-1-yl)propan-1-one O-methyloxime and

### (1Z)-1-phenyl-3-(4-pyridin-2-ylpiperazin-1-yl)propan-1-one O-methyloxime

### EXAMPLE 24A

### (1E)-3-chloro-1-phenylpropan-1-one O-methyloxime and

### (1Z)-3-chloro-1-phenylpropan-1-one O-methyloxime

3′-Chloro-propiophenone (3.4 g, 20 mmol), O-methylhydroxylamine hydrochloride (2.6 g, 30 mmol), and sodium acetate trihydrate (4.2 g, 30 mmmol) were combined in 1,4-dioxane (20 mL), methanol (5 mL) and H₂O (7 mL) and stirred at ambient temperature for 24 hours. The organics were removed under reduced pressure and the residue was extracted with ethyl acetate. The ethyl acetate layers were combined, washed with brine, dried with anhydrous MgSO₄, filtered, and the filtrate was concentrated under reduced pressure. The residue was purified by column chromatography to provide the title compounds. ¹H NMR (300 MHz, DMSO-d₆) δ 2.98 and 3.22 (two t, 5:16, J=7 Hz, 2H), 3.65 and 3.76 (two t, 5:16, J=7 Hz, 2H), 3.74 and 3.93 (two t, 5:16, 3H), 7.44 (m, 3H), 7.67 (m, 2H); MS (DCI/NH₃) m/z 198 (M+H)⁺.

### EXAMPLE 24B

### (1E)-1-phenyl-3-(4-pyridin-2-ylpiperazin-1-yl)propan-1-one O-methyloxime and

### (1Z)-1-phenyl-3-(4-pyridin-2-ylpiperazin-1-yl)propan-1-one O-methyloxime

The products from Example 24A (800 mg, 4 mmol) and 1-(2-pyridyl)piperazine (1.2 mL, 8 mmol) were combined in toluene (40 mL) and refluxed for 14 hours. The mixture was allowed to cool to room temperature and was concentrated under reduced pressure. The residue was purified by column chromatography (hexanes:ethyl acetate 1:1) to provide the title compounds.

E-isomer: maleate salt, mp ° C. ¹H NMR (300 MHz, DMSO-d₆) δ 3.20 (m, 12H), 3.98 (s, 3H), 6.08 (s, 2H), 6.73 (d-d, J=7 Hz, 4 Hz, 1H), 6.95(d, J=9 Hz, 1H), 7.45 (m, 3H), 7.60 (m, 1H), 7.70 (m, 2H), 8.16 (m, 1H); MS (DCI/NH₃) m/z 325 (M+H)⁺. Anal. calcd for C₂₃H₂₈N₄O₅: C, 62.71; H, 6.41; N, 12.72. Found: C, 62.43; H, 6.56; N, 12.47.

Z-isomer: maleate salt, mp ° C. ¹H NMR (300 MHz, DMSO-d₆) δ 2.97 (m, 2H), 3.30 (m, 10H), 3.79 (s, 3H), 6.08 (s, 2H), 6.74 (d-d, J=7 Hz, 4 Hz, 1H), 6.94 (d, J=9 Hz, 1H), 7.45 (m, 5H), 7.60 (m, 1H), 8.16 (m, 1H); MS (DCI/NH₃) m/z 325 (M+H)⁺.

Anal. calcd for C₂₃H₂₈N₄O₅.0.25H₂O: C, 62.08; H, 6.34; N, 12.59. Found: C, 61.80; H, 6.27; N, 12.20.

### EXAMPLE 25

### (1E)-1-phenyl-3-(4-pyridin-2-ylpiperazin-1-yl)propan-1-one O-propyloxime and

### (1Z)-1-phenyl-3-(4-pyridin-2-ylpiperazin-1-yl)propan-1-one O-propyloxime

The title compounds were prepared using the procedure described in Example 23 except using iodopropane instead of iodoethane.

maleate salt, mp 153-154° C. ¹H NMR (300 MHz, DMSO-d₆) δ 0.95 (t, J=7 Hz, 3H), 1.71 (sextet, J=7 Hz, 2H), 3.25 (m, 12H), 4.18 (t, J=7 Hz, 2H), 6.07 (s, 2H), 6.73 (dd, J=4 Hz, 7 Hz, 1H), 6.95 (d, J=9 Hz, 1H), 7.44 (m, 3H), 7.60 (m, 1H), 7.70 (m, 2H), 8.16 (m, 1H); MS (DCI/NH₃) m/z 353 (M+H)⁺. Anal. calcd for C₂₅H₃₂N₄O₅: C, 64.09; H, 6.88; N, 11.96. Found: C, 63.69; H, 6.80; N, 11.83.

### EXAMPLE 26

### (1E)-1-phenyl-3-(4-pyridin-2-ylpiperazin-1-yl)propan-1-one O-allyloxime and

### (1Z)-1-phenyl-3-(4-pyridin-2-ylpiperazin-1-yl)propan-1-one O-allyloxime

The title compounds were prepared using the procedure described in Example 23 except using allyl iodide instead of iodoethane.

E-isomer: mp 136-137° C. ¹H NMR (300 MHz, DMSO-d₆) δ 3.25 (m, 12H), 4.70 (m, 2H), 5.30 (m, 2H), 6.07 (s+m, 3H), 6.73 (dd, J=4 Hz, 7 Hz, 1H), 6.95 (d, J=9 Hz, 1H), 7.44 (m, 3H), 7.60 (m, 1H), 7.70 (m, 2H), 8.16 (m, 1H); MS (DCI/NH₃) m/z 351 (M+H)⁺. Anal. calcd for C₂₁H₂₆N₄O.C₄H₄O₄.0.6H₂O: C, 62.91; H, 6.59; N, 11.74. Found: C, 63.11; H, 6.11; N, 11.27.

Z-isomer: mp 135-137° C. ¹H NMR (300 MHz, DMSO-d₆) δ 1.13 (t, J=7 Hz, 3H), 2.41 (m, 6H), 2.70 (t, J=7 Hz, 2H), 3.42 (t, J=4.5 Hz, 4H), 3.99 (t, J=7 Hz, 2H), 6.61 (dd, J=4 Hz, 7 Hz, 1H), 6.80 (d, J=9 Hz, 1H), 7.40 (m, 5H), 7.70 (m, 1H), 8.09 (m, 1H); MS (DCI/NH₃) m/z 351 (M+H)⁺. Anal. calcd for C₂₁H₂₆N₄O.1.5C₄H₄O₄.4H₂O: C, 49.84; H, 7.13; N, 8.61. Found: C, 49.94; H, 4.91; N, 8.24.

### EXAMPLE 28

### (1E)-1-(3,5-difluorophenyl)-3-(4-pyridin-2-ylpiperazin-1-yl)propan-1-one O-methyloxime and

### (1Z)-1-(3,5-difluorophenyl)-3-(4-pyridin-2-ylpiperazin-1-yl)propan-1-one O-methyloxime

### EXAMPLE 28A

### 1-(3,5-difluorophenyl)-3-(4-pyridin-2-ylpiperazin-1-yl)propan-1-one

The title compound was prepared using the procedure described in Example 1A except using 3,5-difluoroacetophenone instead of 3-chloroacetophenone. MS (DCI/NH₃) m/z 332(M+H)⁺.

### EXAMPLE 28B

### (1E)-1-(3,5-difluorophenyl)-3-(4-pyridin-2-ylpiperazin-1-yl)propan-1-one O-methyloxime and

### (1Z)-1-(3,5-difluorophenyl)-3-(4-pyridin-2-ylpiperazin-1-yl)propan-1-one O-methyloxime

The title compounds were prepared using the procedure described in Example 1B except using the product from Example 28A instead of the product from Example 1A.

E-isomer: mp 70-73° C. ¹H NMR (300 MHz, DMSO-d₆) δ 2.45 (m, 6H), 2.92 (t, J=7 Hz, 2H), 3.40 (t, J=4 Hz, 4H), 3.94 (s, 3H), 6.62 (d-d, J=7 Hz, 4 Hz, 1H), 6.80 (d, J=9 Hz, 1H), 7.33 (m, 3H), 7.50 (m, 1H), 8.09 (m, 1H); MS (DCI/NH₃) m/z 361 (M+H)⁺.

Anal. calcd for C₁₉H₂₂F₂N₄O.0.3H₂O: C, 62.38; H, 6.23; N, 15.32. Found: C, 62.23; H, 6.34; N, 15.25.

Z-isomer: maleate salt, mp 137-138° C. ¹H NMR (300 MHz, DMSO-d₆) δ 3.00 (m, 2H), 3.23 (m, 10H), 3.80 (s, 3H), 6.07 (s, 2H), 6.73 (dd, J=7 Hz, 4 Hz, 1H), 6.95 (d, J=9 Hz, 1H), 7.30 (m, 3H), 7.60 (m, 1H), 8.16 (m, 1H); (DCI/NH₃) m/z 361 (M+H)⁺.

Anal. calcd for C₂₃H₂₆F₂N₄O₅: C, 57.98; H, 5.50; N, 11.76. Found: C, 57.97; H, 5.53; N, 11.52.

### EXAMPLE 29

### ({[1-phenyl-3-(4-pyridin-2-ylpiperazin-1-yl)propylidene]amino}oxy)acetonitrile

The title compound was prepared using the procedure described in Example 23 except using bromoacetonitrile instead of ethyl iodide.

maleate salt, mp 127-128° C. ¹H NMR (300 MHz, DMSO-d₆) δ 3.30 (m, 12H), 5.13 (s, 2H), 6.07 (s, 2H), 6.73 (dd, J=4 Hz, 7 Hz, 1H), 6.95 (d, J=9 Hz, 1H), 7.50 (m, 3H), 7.60 (m, 1H), 7.73 (m, 2H), 8.16 (m, 1H); MS (DCI/NH₃) m/z 350 (M+H)⁺. Anal. calcd for C₂₄H₂₇N₅O₅: C, 61.92; H, 5.85; N, 15.04. Found: C, 61.67; H, 5.75; N, 14.78.

### EXAMPLE 30

### 1-phenyl-3-(4-pyridin-2-ylpiperazin-1-yl)propan-1-one O-butyloxime

The title compound was prepared using the procedure described in Example 23 except using iodobutane instead of iodoethane.

maleate salt, mp 154-155° C. ¹H NMR (300 MHz, DMSO-d₆) δ 0.95 (t, J=7 Hz, 3H), 1.40 (sextet, J=7 Hz, 2H), 1.68 (q, J=7 Hz, 2H), 3.25 (m, 12H), 4.18 (t, J=7 Hz, 2H), 6.07 (s, 2H), 6.73 (dd, J=4 Hz, 7 Hz, 1H), 6.95 (d, J=9 Hz, 1H), 7.44 (m, 3H), 7.60 (m, 1H), 7.70 (m, 2H), 8.16 (m, 1H); MS (DCI/NH₃) m/z 367 (M+H)⁺. Anal. calcd for C₂₆H₃₄N₄O₅.0.4H₂O: C, 63.76; H, 7.16; N, 11.44. Found: C, 63.85; H, 6.98; N, 11.14.

### EXAMPLE 31

### (1E)-1-phenyl-3-(4-pyridin-2-ylpiperazin-1-yl)propan-1-one O-isopropyloxime

The title compound was prepared using the procedure described in Example 23 except using isopropyl iodide instead of iodoethane.

maleate salt, mp 156-157° C. ¹H NMR (300 MHz, DMSO-d₆) δ 1.28 (d, J=7 Hz, 6H), 3.25 (m, 12H), 4.42 (q, J=7 Hz, 1H), 6.07 (s, 2H), 6.73 (dd, J=4 Hz, 7 Hz, 1H), 6.95 (d, J=9 Hz, 1H), 7.44 (m, 3H), 7.60 (m, 1H), 7.70 (m, 2H), 8.16 (m, 1H); MS (DCI/NH₃) m/z 353 (M+H)⁺. Anal. calcd for C₂₅H₃₂N₄O₅.0.7H₂O: C, 62.41; H, 7.00; N, 11.64. Found: C, 62.25; H, 6.58; N, 11.51.

### EXAMPLE 32

### (1E)-1-(3,5-dimethylphenyl)-3-(4-pyridin-2-ylpiperazin-1-yl)propan-1-one O-methyloxime and

### (1Z)-1-(3,5-dimethylphenyl)-3-(4-pyridin-2-ylpiperazin-1-yl)propan-1-one O-methyloxime

### EXAMPLE 32A

### 1-(3,5-dimethylphenyl)-3-(4-pyridin-2-ylpiperazin-1-yl)propan-1-one

The title compound was prepared using the procedure described in Example 1A except using 3,5-dimethylacetophenone instead of 3-chloroacetophenone. ¹H NMR (300 MHz, DMSO-d₆) δ 2.33 (s, 6H), 2.44 (t, J=4 Hz, 4H), 2.70 (t, J=7 Hz, 2H), 3.20 (t, J=7 Hz, 2H), 3.44 (t, J=4 Hz, 4H), 6.62 (d-d, J=7 Hz, 4 Hz, 1H), 6.80 (d, J=9 Hz, 1H), 7.28 (m, 1H), 7.51 (m, 1H), 7.60 (m, 2H), 8.10 (m, 1H); MS (DCI/NH₃) m/z 324(M+H)⁺.

### EXAMPLE 32B

### (1E)-1-(3,5-dimethylphenyl)-3-(4-pyridin-2-ylpiperazin-1-yl)propan-1-one O-methyloxime and

### (1Z)-1-(3,5-dimethylphenyl)-3-(4-pyridin-2-ylpiperazin-1-yl)propan-1-one O-methyloxime

The title compounds were prepared using the procedure described in Example 1B except using the product from Example 32A instead of the product from Example 1A.

E-isomer: maleate salt, mp 167-168° C. ¹H NMR (300 MHz, DMSO-d₆) δ 2.30 (s, 6H), 3.20 (m, 12H), 3.95 (s, 3H), 6.07 (s, 2H), 6.73 (d-d, J=7 Hz, 4 Hz, 1H), 6.95 (d, J=9 Hz, 1H), 7.08 (m, 1H), 7.28 (m, 2H), 7.60 (m, 1H), 8.16 (m, 1H); MS (DCI/NH₃) m/z 353 (M+H)⁺. Anal. calcd for C₂₄H₃₂N₄O₅.0.6H₂O: C, 62.64; H, 6.98; N, 11.69. Found: C, 62.46; H, 6.85; N, 11.45.

Z-isomer: maleate salt, mp 131-133° C. ¹H NMR (300 MHz, DMSO-d₆) δ 2.30 (s, 6H), 2.96 (m, 2G), 3.30 (m, 10H), 3.77 (s, 3H), 6.07 (s, 3H), 6.75 (d-d, J=7 Hz, 4 Hz, 1H), 6.95 (d, J=9 Hz, 1H), 7.08 (m, 3H), 7.28 (m, 2H), 7.60 (m, 1H), 8.16 (m, 1H); (DCI/NH₃) m/z 353 (M+H)⁺. Anal. calcd for C₂₄H₃₂N₄O₅.1.5H₂O: C, 61.58; H, 6.51; N, 10.64. Found: C, 61.19; H, 6.6.54; N, 10.44.

### EXAMPLE 33

### (1E)-1-(4-chloro-3-methylphenyl)-3-(4-pyridin-2-ylpiperazin-1-yl)propan-1-one O-methyloxime and

### (1Z)-1-(4-chloro-3-methylphenyl)-3-(4-pyridin-2-ylpiperazin-1-yl)propan-1-one O-methyloxime

### EXAMPLE 33A

### 1-(4-chloro-3-methylphenyl)-3-(4-pyridin-2-ylpiperazin-1-yl)propan-1-one

The title compound was prepared using the procedure described in Example 1A except using 4-chloro-3-methylacetophenone instead of 3-chloroacetophenone. ¹H NMR (300 MHz, DMSO-d₆) δ 2.40 (s, 3H), 2.44 (t, J=4 Hz, 4H), 2.72 (t, J=7 Hz, 2H), 3.23 (t, J=7 Hz, 2H), 3.44 (t, J=4 Hz, 4H), 6.62 (d-d, J=7 Hz, 4 Hz, 1H), 6.80 (d, J=9 Hz, 1H), 7.53 (m, 2H), 7.63 (m, 1H), 8.00 (m, 1H), 8.10 (m, 1H); MS (DCI/NH₃) m/z 344 (M+H)⁺.

### EXAMPLE 33B

### (1E)-1-(4-chloro-3-methylphenyl)-3-(4-pyridin-2-ylpiperazin-1-yl)propan-1-one O-methyloxime and

### (1Z)-1-(4-chloro-3-methylphenyl)-3-(4-pyridin-2-ylpiperazin-1-yl)propan-1-one O-methyloxime

The title compounds were prepared using the procedure described in Example 1B except using the product from Example 32A instead of the product from Example 1A.

E-isomer: maleate salt, mp 177-178° C. ¹H NMR (300 MHz, DMSO-d₆) δ 2.37 (s, 3H), 3.25 (m, 12H), 3.96 (s, 3H), 6.06 (s, 2H), 6.73 (d-d, J=7 Hz and 4 Hz, 1H), 6.95 (d, J=9 Hz, 1H), 7.60 (m, 4H), 8.16 (m, 1H); MS (DCI/NH₃) m/z 373 (M+H)⁺. Anal. calcd for C₂₄H₂₉ClN₄O₅.0.6H₂O: C, 57.68; H, 6.09; N, 11.21. Found: C, 57.38; H, 5.95; N, 10.94.

Z-isomer: maleate salt, mp 136-137° C. ¹H NMR (300 MHz, DMSO-d₆) δ 2.37 (s, 3H), 3.00 (m, 2H), 3.30 (m, 10H), 3.80 (s, 3H), 6.06 (s, 2H), 6.73 (d-d, J=7 Hz and 4 Hz, 1H), 6.95 (d, J=9 Hz, 1H), 7.36 (m, 1H), 7.50 (m, 2H), 7.60 (m, 1H), 8.16 (m, 1H); MS (DCI/NH₃) m/z 373 (M+H)⁺. Anal. calcd for C₂₄H₂₉ClN₄O₅.0.6H₂O: C, 57.68; H, 6.09; N, 11.21. Found: C, 58.01; H, 5.82; N, 10.74.

### EXAMPLE 34

### (1E)-1-(2-naphthyl)-2-(4-pyridin-2-ylpiperazin-1-yl)ethanone O-methyloxime and

### (1Z)-1-(2-naphthyl)-2-(4-pyridin-2-ylpiperazin-1-yl)ethanone O-methyloxime

### EXAMPLE 34A

### 1-(2-naphthyl)-2-(4-pyridin-2-ylpiperazin-1-yl)ethanone

The title compound was prepared using the procedure described in Example 3A except using 2-bromo-2′-acetonaphthone for 4-fluorophenacyl chloride. MS (DCI/NH₃) m/z 332(M+H)⁺.

### EXAMPLE 34B

### (1E)-1-(2-naphthyl)-2-(4-pyridin-2-ylpiperazin-1-yl)ethanone O-methyloxime and

### (1Z)-1-(2-naphthyl)-2-(4-pyridin-2-ylpiperazin-1-yl)ethanone O-methyloxime

The title compounds were prepared using the procedure described in Example 1B except using the product from Example 34A instead of the product from Example 1A.

Z-isomer: ¹H NMR (300 MHz, DMSO-d₆) δ 2.45 (m, 4H), 3.38 (m, 4H), 3.50 (s, 2H), 3.80 (s, 3H), 6.60 (d-d, J=7 Hz and 4 Hz, 1H), 6.75 (d, J=9 Hz, 1H), 7.55 (m, 3H), 7.64 (dd, J=9 Hz, 3 Hz, 1H), 7.93 (m, 3H), 8.07 (m, 1H), 8.11 (m, 1H); MS (DCI/NH₃) m/z 361 (M+H)⁺. Anal. calcd for C₂₂H₂₄N₄O: C, 73.31; H, 6.71; N, 15.54. Found: C, 73.07; H, 6.86; N, 15.49.

E-isomer: ¹H NMR (300 MHz, DMSO-d₆) δ 2.45 (m, 4H), 3.30 (s, 2H), 3.40 (t, J=4 Hz, 4H), 3.97 (s, 3H), 6.60 (d-d, J=7 Hz and 4 Hz, 1H), 6.75 (d, J=9 Hz, 1H), 7.55 (m, 3H), 7.95 (m, 4H), 8.07 (m, 1H), 8.15 (m, 1H); MS (DCI/NH₃) m/z 361 (M+H)⁺.

### EXAMPLE 35

### (1E)-1-(3-methylphenyl)-3-(4-pyridin-2-ylpiperazin-1-yl)propan-1-one O-ethyloxime and

### (1Z)-1-(3-methylphenyl)-3-(4-pyridin-2-ylpiperazin-1-yl)propan-1-one O-ethyloxime

The title compounds were prepared using the procedure described in Example 11B except using the product from Example 7A instead of the product from Example 11A.

E-isomer: ¹H NMR (300 MHz, DMSO-d₆) δ 1.25 (t, J=7 Hz, 3H), 2.33 (s, 3H), 2.30 (m, 6H), 2.91 (m, 2H), 3.45 (t, J=4 Hz, 4H), 4.17 (q, J=7 Hz, 2H), 6.62 (d-d, J=7 Hz and 4 Hz, 1H), 6.80 (d, J=9 Hz, 1H), 7.20 (m, 1H), 7.30 (t, J=9 Hz, 1H), 7.50 (m, 3H), 8.10 (m, 1H); MS (DCI/NH₃) m/z 353 (M+H)⁺. Anal. calcd for C₂₁H₂₈N₄O: C, 71.32; H, 8.06; N, 15.90. Found: C, 71.56; H, 8.01; N, 15.90.

Z-isomer: ¹H NMR (300 MHz, DMSO-d₆) δ 1.17 (t, J=7 Hz, 3H), 2.33 (s, 3H), 2.40 (m, 6H), 2.70 (t, J=7 Hz, 2H), 3.41 (t, J=4 Hz, 4H), 3.98 (q, J=7 Hz, 2H), 6.62 (d-d, J=7 Hz and 4 Hz, 1H), 6.80 (d, J=9 Hz, 1H), 7.20 (m, 3H), 7.28 (t, J=9 Hz, 1H), 7.50 (m, 1H), 8.10 (m, 1H); MS (DCI/NH₃) m/z 353 (M+H)⁺.

### EXAMPLE 36

### 1-(4-fluorophenyl)-3-(4-pyridin-2-ylpiperazin-1-yl)propan-1-one O-(2,2,2-trifluoroethyl)oxime

The product from Example 21B (328 mg, 1 mmol), CsF (320 mg, 2 mmol), and CF₃CH₂I (230 mg, ˜1.1 mmol) were combined in DMF (10 mL) and refluxed for 12 hours. The mixture was allowed to cool to room temperature, poured into water, and extracted with ethyl acetate. The ethyl acetate extracts were combined, washed with water, brine, dried with anhydrous MgSO₄, filtered, and the filtrate was concentrated under reduced pressure. The residue was purified by column chromatography (silica gel, 1:1 hexanes:ethyl acetate) to provide the title compound.

maleate salt. ¹H NMR (300 MHz, DMSO-d₆) δ 3.20 (m, 12H), 4.95 (q, J=9 Hz, 2H), 6.08 (s, 2H), 6.73 (d-d, J=7 Hz, 4 Hz, 1H), 6.95 (d, J=9 Hz, 1H), 7.33 (t, J=9 Hz, 2H), 7.60 (m, 1H), 7.75 (m, 2H), 8.16 (m, 1H); MS (DCI/NH₃) m/z 411 (M+H)⁺. Anal. calcd for C₂₄H₂₆F₄N₄O₅: C, 54.75; H, 4.98; N, 10.64. Found: C, 54.26; H, 4.86; N, 10.30.

### EXAMPLE 38

### 1-(4-chlorophenyl)-3-(methoxyamino)-2-[(4-pyridin-2-ylpiperazin-1-yl)methyl]propan-1-one O-methyloxime and

### 1-(4-chlorophenyl)-3-isopropoxy-2-[(4-pyridin-2-ylpiperazin-1-yl)methyl]propan-1-one O-methyloxime

### EXAMPLE 38A

### 1-(4-chlorophenyl)-2-[(4-pyridin-2-ylpiperazin-1-yl)methyl]prop-2-en-1-one and

### 1-(4-chlorophenyl)-3-isopropoxy-2-[(4-pyridin-2-ylpiperazin-1-yl)methyl]propan-1-one

The title compounds were prepared using the procedure described in Example 1A except using 3,4′-dichloroacetophenone instead of 3-chloroacetophenone. MS (DCI/NH₃) m/z 342 (M+H)⁺ and m/z 402 (M+H)⁺.

### EXAMPLE 38B

### 1-(4-chlorophenyl)-3-(methoxyamino)-2-[(4-pyridin-2-ylpiperazin-1-yl)methyl]propan-1-one O-methyloxime and

### 1-(4-chlorophenyl)-3-isopropoxy-2-[(4-pyridin-2-ylpiperazin-1-yl)methyl]propan-1-one O-methyloxime

The title compounds were prepared using the procedure described in Example 1B except using the products from Example 38A instead of the product from Example 1A

1:1 mixture of Z:E isomers, di-maleate salt. Mp 118-121° C. ¹H NMR (300 MHz, DMSO-d₆) δ 3.20 (m+2s, 13H), 3.70 (m+2s, 6H), 6.18 (s, 4H), 6.75 (m, 1H), 6.95 (m, 1H), 7.50 (m, 3H), 7.60 (m, 1H), 7.75 (m, 1H), 8.16 (m, 1H); MS (DCI/NH₃)m/z 418 (M+H)⁺. Anal. calcd for C₂₁H₂₈ClN₅O₂.1.75C₄H₄O₄: C, 54.15; H, 5.68; N, 11.28. Found: C, 54.50; H, 5.33; N, 10.60.

2:1 mixture of Z:E isomers, di-maleate salt. Mp 106-109° C. ¹H NMR (300 MHz, DMSO-d₆) δ 1.01 (m, 6H), 3.40 (m, 20H), 3.80 (s, 2H), 3.92 (s, 1H), 6.18 (s, 4H), 6.75 (m, 1H), 6.95 (m, 1H), 7.50 (m, 5H), 8.16 (m, 1H); MS (DCI/NH₃) m/z 431 (M+H)⁺. Anal. calcd for C₃₁H₃₉ClN₄O₁₀: C, 56.15; H, 5.93; N, 8.45. Found: C, 56.07; H, 5.62; N, 8.34.

### EXAMPLE 39

### 1-(4-chlorophenyl)-2-methyl-3-(4-pyridin-2-ylpiperazin-1-yl)propan-1-one O-methyloxime

### EXAMPLE 39A

### 1-(4-chlorophenyl)-2-methyl-3-(4-pyridin-2-ylpiperazin-1-yl)propan-1-one

The title compound was prepared using the procedure described in Example 1A except using 4′-chloro-2-propiophenone instead of 3-chloroacetophenone. MS (DCI/NH₃) m/z 344 (M+H)⁺.

### EXAMPLE 39B

### 1-(4-chlorophenyl)-2-methyl-3-(4-pyridin-2-ylpiperazin-1-yl)propan-1-one O-methyloxime

The title compound was prepared using the procedure described in Example 1B except using the product from Example 39A instead of the product from Example 1A.

di-maleate salt, mp 152-153° C. ¹H NMR (300 MHz, DMSO-d₆) δ 1.05 and 1.28 (2d,3:1, J=7 Hz, 3H), 3.30 (m, 11H), 3.78 and 3.92 (2s, 3:1, 3H), 6.18 (s, 4H), 6.73 (m, 1H), 6.93 (m, 1H), 7.50 (m, 5H), 8.16 (m, 1H); MS (DCI/NH₃) m/z 373 (M+H)⁺.

Anal. calcd for C₂₈H₃₃ClN₄O₉: C, 55.58; H, 5.50; N, 9.26. Found: C, 55.70; H, 5.41; N, 9.24.

### EXAMPLE 40

### (1E)-1-(3,4-dichlorophenyl)-3-(4-pyridin-2-ylpiperazin-1-yl)propan-1-one O-methyloxime and

### (1Z)-1-(3,4-dichlorophenyl)-3-(4-pyridin-2-ylpiperazin-1-yl)propan-1-one O-methyloxime

### EXAMPLE 40A

### 1-(3,4-dichlorophenyl)-3-(4-pyridin-2-ylpiperazin-1-yl)propan-1-one

The title compound was prepared using the procedure described in Example 1A except using 3,4-dichloro-acetophenone instead of 3-chloroacetophenone. MS (DCI/NH₃) m/z 364 (M+H)⁺.

### EXAMPLE 40B

### (1E)-1-(3,4-dichlorophenyl)-3-(4-pyridin-2-ylpiperazin-1-yl)propan-1-one O-methyloxime and

### (1Z)-1-(3,4-dichlorophenyl)-3-(4-pyridin-2-ylpiperazin-1-yl)propan-1-one O-methyloxime

The title compounds were prepared using the procedure described in Example 1B except using the product from Example 40A instead of the product from Example 1A.

E-isomer: maleate salt, mp 182-183° C. ¹H NMR (300 MHz, DMSO-d₆) δ 3.27 (m, 12H), 3.98 (s, 3H), 6.07 (s, 2H), 6.73 (d-d, J=7 Hz and 4 Hz, 1H), 6.95 (d, J=9 Hz, 1H), 7.60 (m, 1H), 7.70 (m, 2H), 7.90 (d, J=3 Hz, 1H), 8.16 (m, 1H); MS (DCI/NH₃) m/z 393 (M+H)⁺. Anal. calcd for C₂₃H₂₆Cl₂N₄O₅: C, 54.23; H, 5.14; N, 11.00. Found: C, 54.31; H, 4.96; N, 10.63.

Z-isomer: maleate salt, mp 140-142° C. ¹H NMR (300 MHz, DMSO-d₆) δ 3.00 (m, 2H), 3.30 (m, 10H), 3.80 (s, 3H), 6.06 (s, 2H), 6.73 (d-d, J=7 Hz and 4 Hz, 1H), 6.95 (d, J=9 Hz, 1H), 7.50 (m, 1H), 7.60 (m, 1H), 7.75 (d, J=9 Hz,1H), 7.80 (d, J=3 Hz, 1H), 8.16 (m, 1H); MS (DCI/NH₃) m/z 393 (M+H)⁺. Anal. calcd for C₂₃H₂₆Cl₂N₄O₅.0.5H₂O: C, 53.29; H, 5.25; N, 10.81. Found: C, 53.41; H, 4.96; N, 10.43.

### EXAMPLE 41

### (1E)-1-(2-chlorophenyl)-3-(4-pyridin-2-ylpiperazin-1-yl)propan-1-one O-methyloxime and

### (1Z)-1-(2-chlorophenyl)-3-(4-pyridin-2-ylpiperazin-1-yl)propan-1-one O-methyloxime

### EXAMPLE 41A

### 1-(2-chlorophenyl)-3-(4-pyridin-2-ylpiperazin-1-yl)propan-1-one

The title compound was prepared using the procedure described in Example 1A except using 2-chloro-acetophenone instead of 3-chloroacetophenone. MS (DCI/NH₃) m/z 330 (M+H)⁺.

### EXAMPLE 41B

### (1E)-1-(2-chlorophenyl)-3-(4-pyridin-2-ylpiperazin-1-yl)propan-1-one O-methyloxime and

### (1Z)-1-(2-chlorophenyl-3-(4-pyridin-2-ylpiperazin-1-yl)propan-1-one O-methyloxime

The title compounds were prepared using the procedure described in Example 1B except using the product from Example 41A instead of the product from Example 1A.

E-isomer: maleate salt, mp 129-130° C. ¹H NMR (300 MHz, DMSO-d₆) δ 3.30 (m, 12H), 3.93 (s, 3H), 6.09 (s, 2.4H), 6.72 (m, 1 Hz, 1H), 6.90 (d, J=9 Hz, 1H), 7.20 (m, 0.5H), 7.50 (m, 5H), 8.15 (m, 1H); MS (DCI/NH₃) m/z 359 (M+H)⁺. Anal. calcd for C₁₉H₂₃ClN₄O.1.2C₄H₄O₄.0.5H₂O.0.1C₇H₈: C, 56.99; H, 5.78.; N, 10.85. Found: C, 56.59; H, 5.41; N, 10.55.

Z-isomer: maleate salt, mp 113-116° C. ¹H NMR (300 MHz, DMSO-d₆) 2.93 (m, 2H), 3.35 (m, 10H), 3.75 (s, 3H), 6.73 (d-d, J=7 Hz, 4 Hz, 1H), 6.95 (d, J=9 Hz, 1H), 7.32 (m, 1H), 7.42 (m, 2H), 7.60 (m, 2H), 8.16 (m, 1H); MS (DCI/NH₃) m/z 359 (M+H)⁺.

Anal. calcd for C₁₉H₂₃ClN₄O.1.4C₄H₄O₄.0.3H₂O: C, 56.09; H, 5.59.; N, 10.64. Found: C, 55.87; H, 5.35; N, 10.33.

### EXAMPLE 42

### (1E)-1-(2,4-dichlorophenyl)-3-(4-pyridin-2-ylpiperazin-1-yl)propan-1-one O-methyloxime and

### (1Z)-1-(2,4-dichlorophenyl)-3-(4-pyridin-2-ylpiperazin-1-yl)propan-1-one O-methyloxime

### EXAMPLE 42A

### 1-(2,4-dichlorophenyl)-3-(4-pyridin-2-ylpiperazin-1-yl)propan-1-one

The title compound was prepared using the procedure described in Example 1A except using 2,4-dichloro-acetophenone instead of 3-chloroacetophenone. MS (DCI/NH₃) m/z 364 (M+H)⁺.

### EXAMPLE 42B

### (1E)-1-(2,4-dichlorophenyl)-3-(4-pyridin-2-ylpiperazin-1-yl)propan-1-one O-methyloxime and

### (1Z)-1-(2,4-dichlorophenyl)-3-(4-pyridin-2-ylpiperazin-1-yl)propan-1-one O-methyloxime

The title compounds were prepared using the procedure described in Example 1B except using the product from Example 42A instead of the product from Example 1A.

E-isomer: ¹H NMR (300 MHz, DMSO-d₆) δ 2.45 (m, 6H), 2.92 (t, J=7 Hz, 2H), 3.37 (m, 4H), 3.90 (s, 3H), 6.61 (m, 1H), 6.78 (d, J=9 Hz, 1H), 7.50 (m, 3H), 7.71 (s, 1H), 8.10 (m, 1H); MS (DCI/NH₃) m/z 393 (M+H)⁺. Anal. calcd for C₁₉H₂₂Cl₂N₄O.0.25H₂O: C, 57.36.40; H, 5.70; N, 14.08. Found: C, 57.20; H, 5.95; N, 13.81.

Z-isomer: ¹H NMR (300 MHz, DMSO-d₆) δ 2.40 (m, 6H), 2.66 (t, J=7 Hz, 2H), 3.40 (t, J=4.5 Hz, 4H), 3.70 (s, 3H), 6.62 (dd, J=4 Hz, 7 Hz, 1H), 6.80 (d, J=9 Hz, 1H), 7.39 (d, J=9 Hz, 1H), 7.50 (m, 2H), 7.67 (d, J=3 Hz, 1H), 8.10 (m, 1H); MS (DCI/NH₃) m/z 393 (M+H)⁺. Anal. calcd for C₁₉H₂₂Cl₂N₄O: C, 58.02.; H, 5.64; N, 14.24. Found: C, 57.88; H, 5.97; N, 14.24.

### EXAMPLE 43

### (1E)-1-(4-bromophenyl)-3-(4-pyridin-2-ylpiperazin-1-yl)propan-1-one O-methyloxime and

### (1Z)-1-(4-bromophenyl)-3-(4-pyridin-2-ylpiperazin-1-yl)propan-1-one O-methyloxime

### EXAMPLE 43A

### 1-(4-bromophenyl)-3-(4-pyridin-2-ylpiperazin-1-yl)propan-1-one

The title compound was prepared using the procedure described in Example 1A except using 4-bromo-acetophenone instead of 3-chloroacetophenone. MS (DCI/NH₃) m/z 374 (M+H)⁺.

### EXAMPLE 43B

### (1E)-1-(4-bromophenyl)-3-(4-pyridin-2-ylpiperazin-1-yl)propan-1-one O-methyloxime and

### (1Z)-1-(4-bromophenyl)-3-(4-pyridin-2-ylpiperazin-1-yl)propan-1-one O-methyloxime

The title compounds were prepared using the procedure described in Example 1B except using the product from Example 43A instead of the product from Example 1A.

E-isomer: ¹H NMR (300 MHz, DMSO-d₆) δ 2.45 (m, 6H), 2.92 (t, J=7 Hz, 2H), 3.42 (t, J=4.5 Hz, 4H), 3.93 (s, 3H), 6.62 (dd, J=4 Hz, 7 Hz, 1H), 6.80 (d, J=9 Hz, 1H), 7.50 (m, 1H), 7.71 (s, 4H), 8.10 (m, 1H); MS (DCI/NH₃) m/z 403 (M+H)⁺. Anal. calcd for C₁₉H₂₃BrN₄O: C, 56.58.40; H, 5.75; N, 13.89. Found: C, 56.51; H, 5.97; N, 13.82.

Z-isomer: ¹H NMR (300 MHz, DMSO-d₆) δ 2.40 (m, 6H), 2.70 (t, J=7 Hz, 2H), 3.40 (t, J=4.5 Hz, 4H), 3.70 (s, 3H), 6.62 (dd, J=4 Hz, 7 Hz, 1H), 6.80 (d, J=9 Hz, 1H), 7.39 (d, J=9 Hz, 2H), 7.50 (m, 1H), 7.61 (d, J=9 Hz, 2H), 8.10 (m, 1H); MS (DCI/NH₃) m/z 403 (M+H)⁺. Anal. calcd for C₁₉H₂₃BrN₄O: C, 56.58; H, 5.75; N, 13.89. Found: C, 56.58; H, 5.78; N, 13.78.

### EXAMPLE 44

### (1E)-1-(3-fluorophenyl)-3-(4-pyridin-2-ylpiperazin-1-yl)propan-1-one O-methyloxime and

### (1Z)-1-(3-fluorophenyl)-3-(4-pyridin-2-ylpiperazin-1-yl)propan-1-one O-methyloxime

### EXAMPLE 44A

### 1-(3-fluorophenyl)-3-(4-pyridin-2-ylpiperazin-1-yl)propan-1-one

The title compound was prepared using the procedure described in Example 1A except using 3-fluoro-acetophenone instead of 3-chloroacetophenone. MS (DCI/NH₃) m/z 314 (M+H)⁺.

### EXAMPLE 44B

### (1E)-1-(3-fluorophenyl)-3-(4-pyridin-2-ylpiperazin-1-yl)propan-1-one O-methyloxime and

### (1Z)-1-(3-fluorophenyl)-3-(4-pyridin-2-ylpiperazin-1-yl)propan-1-one O-methyloxime

The title compounds were prepared using the procedure described in Example 1B except using the product from Example 44A instead of the product from Example 1A.

E-isomer: maleate salt, mp 157-159° C. ¹H NMR (300 MHz, DMSO-d₆) δ 3.30 (m, 12H), 3.98 (s, 3H), 6.08 (s, 2H), 6.73 (d-d, J=7 Hz, 4 Hz, 1H), 6.95(d, J=9 Hz, 1H), 7.30 (m, 1H), 7.52 (m, 3H), 7.60 (m, 1H), 8.16 (m, 1H); MS (DCI/NH₃) m/z 343 (M+H)⁺. Anal. calcd for C₂₃H₂₇FN₄O₅: C, 60.25; H, 5.91; N, 12.22. Found: C, 59.87; H, 5.86; N, 11.88.

Z-isomer: maleate salt, mp 122-124° C. ¹H NMR (300 MHz, DMSO-d₆) δ 3.00 (m, 2H), 3.30 (m, 10H), 3.79 (s, 3H), 6.08 (s, 2.5H), 6.74 (d-d, J=7 Hz, 4 Hz, 1H), 6.94 (d, J=9 Hz, 1H), 7.33 (m, 3H), 7.50 (m, 1H), 7.60 (m, 1H), 8.16 (m, 1H); MS (DCI/NH₃) m/z 343 (M+H)⁺. Anal. calcd for C₁₉H₂₃FN₄O.1.25C₄H₄O₄.0.4H₂O: C, 58.06; H, 5.89; N, 11.28. Found: C, 58.28; H, 5.75; N, 10.89.

### EXAMPLE 45

### (1E)-1-(4-fluorophenyl)-2-(4-pyrimidin-2-ylpiperazin-1-yl)ethanone oxime and

### (1Z)-1-(4-fluorophenyl)-2-(4-pyrimidin-2-ylpiperazin-1-yl)ethanone oxime

### EXAMPLE 45A

### 1-(4-fluorophenyl)-2-(4-pyrimidin-2-ylpiperazin-1-yl)ethanone

The title compound was prepared using the procedure described in Example 3A except using 1-(2-pyrimidyl)piperazine instead of 1-(2-pyridinyl)piperazine. MS (DCI/NH₃) m/z 301 (M+H)⁺.

### EXAMPLE 45B

### (1E)-1-(4-fluorophenyl)-2-(4-pyrimidin-2-ylpiperazin-1-yl)ethanone oxime and

### (1Z)-1-(4-fluorophenyl)-2-(4-pyrimidin-2-ylpiperazin-1-yl)ethanone oxime

The title compounds were prepared using the procedure described in Example 15B except using the product from Example 45A instead of the product from Example 15A.

Z-isomer: mp 127-128° C., ¹H NMR (300 MHz, DMSO-d₆) δ 2.43 (t, J=4 Hz, 4H), 3.38 (s, 2H), 3.65 (t, J=4 Hz, 4H), 6.60 (t, J=4 Hz, 1H), 7.22 (t, J=9 Hz, 1H), 7.31 (t, J=9 Hz, 1H), 7.72 (m, 1H), 8.00 (m, 1H), 8.33 (d, J=4 Hz, 1H), 11.06 (s, 1H); MS (DCI/NH₃) m/z 316 (M+H)⁺.

E-isomer: mp 100-103° C., ¹H NMR (300 MHz, DMSO-d₆) δ 2.46 (t, J=4 Hz, 4H), 3.65 (m, 6H), 6.60 (t, J=4 Hz, 1H), 7.20 (t, J=9 Hz, 1H), 7.32 (t, J=9 Hz, 1H), 7.72 (m, 1H), 8.00 (m, 1H), 8.33 (d, J=4 Hz, 1H), 11.45 (br s, 1H); MS (DCI/NH₃) m/z 316 (M+H)⁺.

### EXAMPLE 46

### (1E)-1-(4-fluorophenyl)-2-(4-pyridin-2-ylpiperazin-1-yl)ethanone oxime and

### (1Z)-1-(4-fluorophenyl)-2-(4-pyridin-2-ylpiperazin-1-yl)ethanone oxime

The title compounds were prepared using the procedure described in Example 15B except using the product from Example 3A instead of the product from Example 15A.

Z-isomer: mp 136-137° C., ¹H NMR (300 MHz, DMSO-d₆) δ 2.46 (m, 4H), 3.38 (m, 6H), 6.60 (dd, J=7 Hz, 4 Hz, 1H), 6.76 (d, J=9 Hz, 1H), 7.20 (t, J=9 Hz, 2H), 7.50 (m, 1H), 7.62 (m, 2H), 8.09 (m, 1H), 11.05 (s, 1H); MS (DCI/NH₃) m/z 315 (M+H)⁺.

E-isomer: mp 136-138° C., ¹H NMR (300 MHz, DMSO-d₆) δ 2.50 (m, 4H), 3.60 (t, J=4 Hz, 4H), 3.66 (s, 2H), 6.62 (dd, J=& Hz, 4 Hz, 1H), 6.77 (d, J=9 Hz, 1H), 7.20 (t, J=9 Hz, 1H), 7.50 (m, 1H), 7.62 (m, 2H), 8.09 (m, 1H), 11.45 (s, 1H); MS (DCI/NH₃) m/z 315 (M+H)⁺. Anal. calcd for C₁₇H₁₉FN₄O.0.3H₂O: C, 63.85; H, 6.18; N, 17.52. Found: C, 64.27; H, 5.99; N, 17.08.

### EXAMPLE 47

### 1-(4-fluorophenyl)-2-(4-pyridin-2-ylpiperazin-1-yl)ethanone methylhydrazone

The product from Example 3A (63 mg, 0.2 mmol), N-methylhydrazine (0.011 mL, 0.2 mmol) and acetic acid (0.012 mL, 0.2 mmol) were combined in 1,4-dioxane (10 mL) and stirred at room temperature for 16 hours. The mixture was concentrated under reduced pressure and the residue partitioned between saturated NaHCO₃ and ethyl acetate. The ethyl acetate was separated and concentrated under reduced pressure. The residue was purified by column chromatography (ethyl acetate) to provide the title compound. ¹H NMR (300 MHz, DMSO-d₆) δ 2.50 (m, 4H), 2.97 (d, J=4.5 Hz, 3H), 3.48 (t, J=4 Hz, 4H), 3.55 (s, 2H), 6.62 (dd, J=& Hz, 4 Hz, 1H), 6.80 (d, J=9 Hz, 1H), 7.12 (t, J=9 Hz, 2H), 7.50 (m, 1H), 7.62 (m, 3H), 8.10 (m, 1H); MS (DCI/NH₃) m/z 328 (M+H)⁺.

### EXAMPLE 48

### 2-{4-[(3E)-3-(hydroxyimino)-3-phenylpropyl]piperazin-1-yl}nicotinonitrile and

### 2-{4-[(3Z)-3-(hydroxyimino)-3-phenylpropyl]piperazin-1-yl}nicotinonitrile

### EXAMPLE 48A

### 2-[4-(3-oxo-3-phenylpropyl)piperazin-1-yl]nicotinonitrile

3-Chloropropiophenone (3.00 g, 17.8 mmol) and potassium carbonate (2.50 g, 18.1 mmol) were combined in N,N-dimethylformamide (35 mL) teated with 2-piperazin-1-ylnicotinonitrile (3.70 g, 19.7 mmol) and heated at 35° C. After 18 hours, the mixture was transferred to a separatory funnel with ethyl acetate and washed with saturated aqueous sodium bicarbonate, dried (Na₂SO₄), filtered, and the filtrate was concentrated under reduced pressure. The residue was purified by flash column chromatography (silica gel; elution with 1:1 ethyl acetate:hexanes) to provide the title compound. ¹H NMR (300 MHz, DMSO-d₆) δ 2.58 (t, J=5.1 Hz, 4H), 2.74 (t, J=7.5 Hz, 2H), 3.25 (t, J=7.1 Hz, 2H), 3.59 (t, J=4.8 Hz, 4H), 6.91 (dd, J=7.8 Hz, 4.8 Hz, 1H), 7.53 (m, 2H), 7.64 (m, 1H), 8.00 (m, 2H), 8.06 (dd, J=7.8 Hz, 2.0 Hz, 1H), 8.40 (dd, J=5.1 Hz, 2.0 Hz, 1H); MS (DCI/NH₃) m/z 321 (M+H)⁺.

### EXAMPLE 48B

### 2-{4-[(3E)-3-(hydroxyimino)-3-phenylpropyl]piperazin-1-yl}nicotinonitrile and

### 2-{4-[(3Z)-3-(hydroxyimino)-3-phenylpropyl]piperazin-1-yl}nicotinonitrile

The product from Example 48A (1.03 g, 3.21 mmol) and sodium acetate trihydrate (930 mg, 6.83 mmol) were combined in methanol (10 mL) and water (1 mL) at room temperature. The mixture was treated with hydroxylamine hydrochloride (720 mg, 10.4 mmol) and heated at 50° C. After 2 hours, the mixture was cooled, concentrated, and the residue partitioned between water and ethyl acetate. The organic phase was dried (Na₂SO₄), filtered, and the filtrate was concentrated under reduced pressure. The residue was purified by flash column chromatography (silica gel; elution with 1:1 ethyl acetate:hexanes) to provide the title compounds. Z-isomer: ¹H NMR (300 MHz, DMSO-d₆) δ 2.50 (m, 2H), 2.57 (t, J=4.8 Hz, 4H), 2.94 (m, 2H), 3.58 (t, J=4.7 Hz, 4H), 6.91 (dd, J=7.8 Hz, 4.7 Hz, 1H), 7.37 (m, 3H), 7.64 (m, 2H), 8.05 (dd, J=7.5 Hz, 2.0 Hz, 1H), 8.40 (dd, J=4.8 Hz, 2.0 Hz, 1H); MS (DCI/NH₃) m/z 336 (M+H)⁺. Anal. calcd for C₁₉H₂₁N₅O: C, 68.04; H, 6.31; N, 20.88. Found: C, 67.83; H, 6.25; N, 20.83. E-isomer: ¹H NMR (300 MHz, DMSO-d₆) δ 2.46 (m, 6H), 2.69 (t, J=7.1 Hz, 2H), 3.56 (t, J=4.8 Hz, 4H), 6.91 (dd, J=7.5 Hz, 4.8 Hz, 1H), 7.39 (m, 5H), 8.05 (dd, J=7.5 Hz, 2.0 Hz, 1H), 8.39 (dd, J=4.8 Hz, 2.0 Hz, 1H); MS (DCI/NH₃) m/z 336 (M+H)⁺. Anal. calcd for C₁₉H₂₁N₅O.0.20 H₂O: C, 67.32; H, 6.36; N, 20.66. Found: C, 67.30; H, 6.30; N, 20.28.

### EXAMPLE 49

### (2E)-1-phenyl-3-(4-pyridin-2-ylpiperazin-1-yl)acetone O-methyloxime and

### (2Z)-1-phenyl-3-(4-pyridin-2-ylpiperazin-1-yl)acetone O-methyloxime

### EXAMPLE 49A

### 1-chloro-3-phenylacetone

Benzylmagnesium chloride (1.0 M in diethyl ether, 50.0 mL) was treated with 2-chloro-N-methoxy-N-methylacetamide (5.16 g, 37.5 mmol) in tetrahydrofuran (200 mL) at −78° C. drop wise. The reaction was allowed to warm slowly to room temperature overnight and quenched with 1N hydrochloric acid. The layers were separated and the organic phase dried (Na₂SO₄), filtered, and the filtrate was concentrated under reduced pressure. The residue was purified by flash column chromatography (silica gel; elution with hexanes) to provide the title. ¹H NMR (300 MHz, DMSO-d₆) δ 3.87 (s, 2H), 4.62 (s, 2H), 7.25 (m, 5H); MS (DCI/NH₃) m/z 186 (M+NH₄)⁺.

### EXAMPLE 49B

### 1-phenyl-3-(4-pyridin-2-ylpiperazin-1-yl)acetone

The product from Example 49A (2.60 g, 15.4 mmol) and diisopropylethylamine (6 mL) were combined in toluene (100 mL), treated with 1-(2-pyridinyl)piperazine (2.80 mL, 18.3 mmol), and heated at 80° C. After 6 hours, the mixture was cooled, washed with saturated aqueous sodium bicarbonate, dried (Na₂SO₄), filtered, and the filtrate was concentrated under reduced pressure. The residue was purified by flash column chromatography (silica gel; elution with 4:1 hexanes:ethyl acetate then 1:1 hexanes:ethyl acetate) to provide the title. ¹H NMR (300 MHz, CDCl₃) δ 2.56 (t, J=5.4 Hz, 4H), 3.26 (s, 2H), 3.58 (t, J=5.1 Hz, 4H), 3.77 (s, 2H), 6.62 (m, 2H), 7.28 (m, 5H), 7.47 (m, 1H), 8.18 (m, 1H); MS (DCI/NH₃) m/z 296 (M+H)⁺.

### EXAMPLE 49C

### (2E)-1-phenyl-3-(4-pyridin-2-ylpiperazin-1-yl)acetone O-methyloxime and

### (2Z)-1-phenyl-3-(4-pyridin-2-ylpiperazin-1-yl)acetone O-methyloxime

The title compounds, a 2:1 mixture of inseparable isomers, were prepared using the procedure described in Example 1B except using the product from Example 49B instead of the product from Example 1A. ¹H NMR (300 MHz, CDCl₃) δ 2.37 (m, 4H), 2.92 (s, 1.4H), 3.10 (s, 0.6H), 3.42 (m, 4H), 3.55 (s, 0.6H), 3.70 (s, 1.4H), 3.77 (s, 1H), 3.82 (s, 2H), 6.63 (m, 1H), 6.77 (m, 1H), 7.27 (m, 5H), 7.50 (m, 1H), 8.10 (m, 1H); MS (DCI/NH₃) m/z 325 (M+H)⁺. Maleate salt: Anal. calcd for C₁₉H₂₄N₄O.1.0 C₄H₄O₄: C, 62.71; H, 6.41; N, 12.72. Found: C, 62.38; H, 6.38; N, 12.35.

### EXAMPLE 50

### 1-phenyl-3-[4-(1,3-thiazol-2-yl)piperazin-1-yl]propan-1-one oxime

### EXAMPLE 50A

### 1-(1,3-thiazol-2-yl)piperazine

2-Bromothiazole (15 g, 91.4 mmol) and piperazine (15.75 g, 182.9 mmol) in butanol were refluxed for 18 hours. The mixture was allowed to cool to room temperature and concentrated under reduced pressure. The residue was treated with 10% aqueous K₂CO₃ and extracted with ethyl acetate. The ethyl acetate extract was dried over anhydrous Na₂CO₃, filtered, and the filtrate was concentrated under reduced pressure to provide the title compound. ¹H NMR (300 MHz, CDCl₃) δ 3.0 (m, 4H), 3.45 (m, 4H), 6.58 (d, J=3 Hz, 1H), 7.2 (d, J=3 Hz, 1H); MS (DCI/NH₃) m/z 170 (M+H)⁺.

### EXAMPLE 50B

### 1-phenyl-3-[4-(1,3-thiazol-2-yl)piperazin-1-yl]propan-1-one

3-Chloropropiophenone (8 g, 47.44 mmol), the product from Example 50A (8.83 g, 52.18 mmol), and K₂CO₃ (6.56 g, 47.44 mmol) were combined in DMF and heated at 35° C. for 16 hours. The mixture was allowed to cool to room temperature, diluted with water, and extracted with ethyl acetate. The ethyl acetate extract was dried over anhydrous Na₂CO₃, filtered, and the filtrate was concentrated under reduced pressure. The residue was purified by column chromatography (silica gel, 1:1 ethyl acetate:hexanes) to provide the title compound. ¹H NMR (300 MHz, CDCl₃) δ 2.65 (br.s, 4H), 2.95 (br.s, 2H), 3.22 (br.s. 2H), 3.22 (br.s, 2H), 3.45 (br.s, 4H), 6.45 (d, J=3 Hz, 1H), 7.2 (d, J=3 Hz, 1H), 7.45 (m, 2H), 7.6 (m,1H), 7.9 (m, 2H); MS (DCI/NH₃) m/z 302 (M+H)⁺. Anal. calcd for C₁₆H₁₉N₃OS: C, 63.76; H, 6.35; N, 13.94. Found: C, 61.50; H, 6.43; N, 13.21.

### EXAMPLE 50C

### 1-phenyl-3-[4-(1,3-thiazol-2-yl)piperazin-1-yl]propan-1-one oxime

The product from Example 50B (500 mg, 1.66 mmol), NH₂OH.HCl (576.8 mg, 8.3 mmol), and sodium acetate (1.13 g, 8.3 mmol) were combined in 80% EtOH/water and heated at 60° C. for 4 hours. The mixture was allowed to cool to room temperature, concentrated under reduced pressure, and the residue purified by column chromatography (silica gel, 1:1 ethyl acetate:hexanes) to provide the title compound. ¹H NMR (300 MHz, Acetone-d₆) δ 2.2 (m, 4H), 2.6 (m, 4H), 3.1 (m, 2H), 4.0 (m, 2H), 6.7 (d, J=3 Hz, 1H), 7.5 (d, J=3 Hz, 1H), 7.35 (m, 3H), 7.7 (m, 2H). MS (DCI/NH₃) m/z 317 (M+H)⁺. Anal. calcd for C₁₆H₂₀N₄OS: C, 60.73; H, 6.37; N, 17.71. Found: C, 60.98; H, 6.37; N, 17.50.

### EXAMPLE 51

### 3-[4-(2-methoxyphenyl)piperazin-1-yl]-1-phenylpropan-1-one O-ethyloxime

### EXAMPLE 51A

### 3-[4-(2-methoxyphenyl)piperazin-1-yl]-1-phenylpropan-1-one

The title compound was prepared using the procedure described in Example 50B except using 1-(2-methoxyphenyl)piperazine instead of the product from Example 50A. MS (DCI/NH₃) m/z 325 (M+H)⁺.

### EXAMPLE 51B

### 3-[4-(2-methoxyphenyl)piperazin-1-yl]-1-phenylpropan-1-one oxime

The title compound was prepared using the procedure described in Example 50C except using the product from Example 51A instead of the product from Example 50B. MS (DCI/NH₃) m/z 340 (M+H)⁺.

### EXAMPLE 51C

### 3-[4-(2-methoxyphenyl)piperazin-1-yl]-1-phenylpropan-1-one O-ethyloxime

The product from Example 51B (380 mg, 1.12 mmol) and potassium tert-butoxide (138 mg, 1.23 mmol) were combined in tert-butanol (15 mL) and refluxed for 30 minutes. The mixture was allowed to cool to 50° C. and was treated with ethyl iodide and refluxed for 1 hour. The mixture was allowed to cool to room temperature and concentrated under reduced pressure. The residue was partitioned between ethyl acetate and brine. The organic phase was separated, dried over MgSO₄, filtered, and the filtrate was concentrated under reduced pressure. The residue was purified by column chromatography (silica gel, 50% ethyl acetate-hexane) to provide the title compound. Maleate salt, mp ° C. ¹H NMR (300 MHz, CDCl₃) δ 1.3 (t, J=7.5 Hz, 3H), 2.7 (m, 6H), 3.1 (m, 6H), 3.82 (s, 3H), 4.25 (q, J=7.5 Hz, 2H), 6.9 (m, 4H), 7.4 (m, 3H), 7.7 (m, 2H); MS (DCI/NH₃) m/z 368 (M+H)⁺. Anal. calcd for C₂₆H₃₃N₃O₆: C, 64.58; H, 6.88; N, 8,69. Found: C, 65.09; H, 7.02; N, 8.77.

### EXAMPLE 52

### 3-[4-(3-methoxyphenyl)piperazin-1-yl]-1-phenylpropan-1-one O-ethyloxime

### EXAMPLE 52A

### 3-[4-(3-methoxyphenyl)piperazin-1-yl]-1-phenylpropan-1-one O-ethyloxime

The title compound was prepared using the procedure described in Example 50B except using 1-(3-methoxyphenyl)piperazine instead of 1-thiazol-2-ylpiperazine. MS (DCI/NH₃) m/z 325 (M+H)⁺.

### EXAMPLE 52B

### 3-[4-(3-methoxyphenyl)piperazin-1-yl]-1-phenylpropan-1-one oxime

The title compound was prepared using the procedure described in Example 50C except using the product from Example 52A instead of the product from Example 50B. MS (DCI/NH₃) m/z 340 (M+H)⁺.

### EXAMPLE 52C

### 3-[4-(3-methoxyphenyl)piperazin-1-yl]-1-phenylpropan-1-one O-ethyloxime

The title compound was prepared using the procedure described in Example 51C except using the product from Example 52B instead of the product from Example 51B. maleate salt, mp ° C. ¹H NMR (300 MHz, DMSO-d₆) δ 1.3 (t, J=7.5 Hz, 3H), 2.5 (m, 6H), 2.9 (m, 2H), 3.10 (m, 4H), 3.65 (s, 3H), 4.2 (q, J=7.5 Hz, 2H), 6.4 (m, 3H), 7.1 (t, J=7.5 Hz, 1H), 7.4 (m, 3H), 7.65 (m, 2H); MS (DCI/NH₃) m/z 368 (M+H)⁺.

Anal. calcd for C₂₆H₃₃N₃O₆: C, 64.58; H, 6.88; N, 8.69. Found: C, 64.37; 6.96; 8.52.

### EXAMPLE 53

### 3-[4-(4-methoxyphenyl)piperazin-1-yl]-1-phenylpropan-1-one O-ethyloxime

### EXAMPLE 53A

### 3-[4-(4-methoxyphenyl)piperazin-1-yl]-1-phenylpropan-1-one

The title compound was prepared using the procedure described in Example 50B except using 1-(4-methoxyphenyl)piperazine instead of 1-thiazol-2-ylpiperazine. MS (DCI/NH₃) m/z 325 (M+H)⁺.

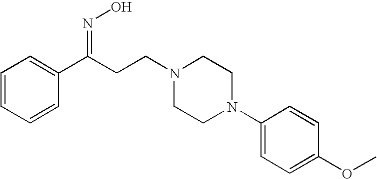

### EXAMPLE 53B

### 3-[4-(4-methoxyphenyl)piperazin-1-yl]-1-phenylpropan-1-one oxime

The title compound was prepared using the procedure described in Example 50C except using the product from Example 53A instead of the product from Example 50B. MS (DCI/NH₃) m/z 340 (M+H)⁺.

### EXAMPLE 53C

### 3-[4-(4-methoxyphenyl)piperazin-1-yl]-1-phenylpropan-1-one O-ethyloxime

The title compound was prepared using the procedure described in Example 50C except using the product from Example 53B instead of the product from Example 50B. maleate salt, mp ° C. ¹H NMR (300 MHz, DMSO-d₆) δ 1.3 (t, J=7.5 Hz, 3H), 2.5 (m, 6H), 3.0 (m, 6H), 3.0 (m, 6H), 3.65 (s, 3H), 4.2 (q, J=7.5 Hz, 2H), 6.8 (m, 4 h), 7.4 (m, 3H), 7.62 (m, 2H); MS (DCI/NH₃) m/z 368 (M+H)⁺. Anal. Calcd for C₂₂H₂₉N₃O₂.1.2C₄H₄O₄.0.3H₂O, C,62.85; H, 6.77; N, 8.20. Found: C, 63.02; H, 6.82; N, 8.02.

### EXAMPLE 54

### 1-phenyl-3-(4-pyrimidin-2-ylpiperazin-1-yl)propan-1-one O-ethyloxime

### EXAMPLE 54A

### 1-phenyl-3-(4-pyrimidin-2-ylpiperazin-1-yl)propan-1-one

The title compound was prepared using the procedure described in Example 50B except using 1-(2-pyrimidyl)piperazine instead of 1-thiazol-2-ylpiperazine. MS (DCI/NH₃) m/z 298 (M+H)⁺.

### EXAMPLE 54B

### 1-phenyl-3-(4-pyrimidin-2-ylpiperazin-1-yl)propan-1-one oxime

The title compound was prepared using the procedure described in Example 50C except using the product from Example 54A instead of the product from Example 50B. MS (DCI/NH₃) m/z 312 (M+H)⁺.

### EXAMPLE 54C

### 1-phenyl-3-(4-pyrimidin-2-ylpiperazin-1-yl)propan-1-one O-ethyloxime

The title compound was prepared using the procedure described in Example 51C except using the product from Example 54B instead of the product from Example 51B. maleate salt, mp ° C. ¹H NMR (300 MHz, CDCl₃) δ 1.3 (t, J=7.5 Hz, 3H), 2.6 (m, 6H), 3.0 (m, 2H), 3.8 (m, 4H), 4.23 (q, J=7.5 Hz, 2H), 6.45 (t, J=6 Hz, 1H), 7.35 (m, 3H), 7.65 (m, 2H), 8.3 (d, J=6 Hz, 2H); MS (DCI/NH₃) m/z 340 (M+H)⁺. Anal. calcd for C₂₃H₂₉N₅O₅: C,60.65; H, 6.42; N, 15.37. Found: C, 60.36; H, 6.32; N, 15.07.

### EXAMPLE 55

### 1-phenyl-3-[4-(1,3-thiazol-2-yl)piperazin-1-yl]propan-1-one O-ethyloxime

The title compound was prepared using the procedure described in Example 51C except using the product from Example 50C instead of the product from Example 51B. maleate salt, mp ° C. ¹H NMR (300 MHz, DMSO-d6) δ 1.25 (t, J=7.5 Hz, 3H), 2.5 (m, 6H), 2.95 (m, 2H), 3.4 (m, 4H), 4.20 (q, J=7.5 Hz, 2H), 6.82 (d, J=3 Hz, 1H), 7.18 (d, J=3 Hz, 1H), 7.4 (m, 3H), 7.7 (m, 2H); MS (DCI/NH₃) m/z 345 (M+H)⁺.

Anal. calcd for C₂₂H₂₈N₄O₅S: C, 54.62; H, 6.14; N, 11.08. Found: C, 54.75; H, 6.16; N, 10.18

### EXAMPLE 56

### 3-[4-(2-ethoxyphenyl)piperazin-1-yl]-1-phenylpropan-1-one O-ethyloxime

### EXAMPLE 56A

### 3-[4-(2-ethoxyphenyl)piperazin-1-yl]-1-phenylpropan-1-one

The title compound was prepared using the procedure described in Example 50B except using 1-(2-ethoxyphenyl)piperazine instead of 1-thiazol-2-ylpiperazine. MS (DCI/NH₃) m/z 339 (M+H)⁺.

### EXAMPLE 56B

### (1E)-3-[4-(2-ethoxyphenyl)piperazin-1-yl]-1-phenylpropan-1-one oxime

The title compound was prepared using the procedure described in Example 50C except using the product from Example 56A instead of the product from Example 50B. MS (DCI/NH₃) m/z 354 (M+H)⁺.

### EXAMPLE 56C

### 3-[4-(2-ethoxyphenyl)piperazin-1-yl]-1-phenylpropan-1-one O-ethyloxime

The title compound was prepared using the procedure described in Example 51C except using the product from Example 56B instead of the product from Example 51B. maleate salt, mp ° C. ¹H NMR (300 MHz, CDCl₃) δ 1.3 (t, J=7.5 Hz, 3H), 2.7 (m, 6H), 3.0 (m, 2H), 3.18 (br. s, 4H), 4.25 (q, J=7.5 Hz, 2H), 6.9 (m, 4H), 7.38 (m, 3H), 7.7 (m, 2H); MS (DCI/NH₃) m/z 382 (M+H)⁺.

Anal. calcd for C₂₇H₃₅N₃O₆: C,65.17; H, 7.09; N, 8.44. Found: C, 64.84; H, 6.90; N, 8.23.

### EXAMPLE 57

### 3-[4-(3-methylpyridin-2-yl)piperazin-1-yl]-1-phenylpropan-1-one O-ethyloxime

### EXAMPLE 57A

### 3-[4-(3-methylpyridin-2-yl)piperazin-1-yl]-1-phenylpropan-1-one

The title compound was prepared using the procedure described in Example 50B except using 1-(2-methylpyridinyl)piperazine instead of 1-thiazol-2-ylpiperazine. MS (DCI/NH₃) m/z 310 (M+H)⁺.

### EXAMPLE 57B

### 3-[4-(3-methylpyridin-2-yl)piperazin-1-yl]-1-phenylpropan-1-one oxime

The title compound was prepared using the procedure described in Example 50C except using the product from Example 57A instead of the product from Example 50B. MS (DCI/NH₃) m/z 325 (M+H)⁺.

### EXAMPLE 57C

### 3-[4-(3-methylpyridin-2-yl)piperazin-1-yl]-1-phenylpropan-1-one O-ethyloxime

The title compound was prepared using the procedure described in Example 51C except using the product from Example 57B instead of the product from Example 51B. maleate salt, mp ° C. MS (DCI/NH₃) m/z 353 (M+H)⁺. Anal. calcd for C₂₅H₃₂N₄O₅: C,64.09; H, 6.88; N, 11.96. Found: C, 64.01; H, 6.95; N, 11.85.

### EXAMPLE 58

### 1-phenyl-3-(4-phenylpiperazin-1-yl)propan-1-one O-ethyloxime

### EXAMPLE 58A

### 1-phenyl-3-(4-phenylpiperazin-1-yl)propan-1-one

The title compound was prepared using the procedure described in Example 50B except using 1-(phenyl)piperazine instead of 1-thiazol-2-ylpiperazine. MS (DCI/NH₃) m/z 295 (M+H)⁺.

### EXAMPLE 58B

### 1-phenyl-3-(4-phenylpiperazin-1-yl)propan-1-one oxime

The title compound was prepared using the procedure described in example 50C except using the product from Example 58A instead of the product from Example 50B. MS (DCI/NH₃) m/z 310 (M+H)⁺.

### EXAMPLE 58C

### 1-phenyl-3-(4-phenylpiperazin-1-yl)propan-1-one O-ethyloxime

The title compound was prepared using the procedure described in Example 51C except using the product from Example 58B instead of the product from Example 51B. maleate salt, mp ° C. ¹H NMR (300 MHz, CDCl₃) δ 1.25 (t, J=6 Hz, 3H), 2.65 (m, 6H), 3.0 (m, 2H), 3.2 (m, 4H), 4.25 (q, 6 Hz, 2H), 6.85 (m, 1H), 6.95 (d, J=7.5 Hz, 2H), 7.3 (m, 2H), 7.4 (m, 3H), 7.65 (m, 2H); MS (DCI/NH₃) m/z 338 (M+H)⁺. Anal. calcd for C₂₅H₃₁N₃O₅: C,66.21; H, 6.89; N, 9.27. Found: C, 66.16; H, 6.77; N, 9.19.

### EXAMPLE 59

### 2-{4-[3-(ethoxyimino)-3-phenylpropyl]piperazin-1-yl}benzonitrile

### EXAMPLE 59A

### 2-[4-(3-oxo-3-phenylpropyl)piperazin-1-yl]benzonitrile

The title compound was prepared using the procedure described in Example 50B except using 1-(2-cyanophenyl)piperazine instead of 1-thiazol-2-ylpiperazine. MS (DCI/NH₃) m/z 320 (M+H)⁺.

### EXAMPLE 59B

### 2-{4-[3-(hydroxyimino)-3-phenylpropyl]piperazin-1-yl}benzonitrile

The title compound was prepared using the procedure described in example 50C except using the product from Example 59A instead of the product from Example 50B. MS (DCI/NH₃) m/z 335 (M+H)⁺.

### EXAMPLE 59C

### 2-{4-[3-(ethoxyimino)-3-phenylpropyl]piperazin-1-yl}benzonitrile

The title compound was prepared using the procedure described in Example 51C except using the product from example 59B instead of the product from Example 51B. maleate salt, mp ° C. ¹H NMR (300 MHz, CDCl₃) δ 1.3 (t, J=7.5 Hz, 3H), 2.7 (m, 6H), 3.0 (br.s, 2H), 3.25 (br.s, 4H), 4.25 (q, J=7.5 Hz, 2H), 7.0 (m, 2H), 7.35 (m, 4H), 7.45 (m, 1H), 7.55 (d-d, J=9 Hz, 3 Hz, 1H), 7.7 (m, 2H); MS (DCI/NH₃) m/z 363 (M+H)⁺. Anal. calcd for C₂₆H₃₀N₄O₅: C,65.26; H,6.32; N, 11.71. Found: C, 65.32; H, 6.39; N, 11.58.

### EXAMPLE 61

### 2-{4-[3-(ethoxyimino)-3-phenylpropyl]piperazin-1-yl}nicotinonitrile

The title compound was prepared using the procedure described in Example 51C except using the product from example 48A instead of the product from Example 51B. maleate salt, mp ° C. ¹H NMR (300 MHz, CDCl₃) δ 1.3 (t, J=7.5 Hz, 3H), 2.65 (br.s, 6H), 3.0 (m, 2H), 3.75 (br.s, 4H), 4.25 (q, J=7.5 Hz, 2H), 6.7 (m, 1H), 7.4 (d-d, J=6 Hz, 3 Hz, 3H), 7.65 (m, 2H), 7.75 (d-d, J=9 Hz, 3 Hz, 1H), 8.35 (d-d, J=6 Hz, 3 Hz, 1H); MS (DCI/NH₃) m/z 364 (M+H)⁺. Anal. calcd for C₂₅H₂₉N₅O₅: C,62.62; H, 6.10; N, 14.60. Found: C, 62.69; H, 6.22; N, 14.72.

### EXAMPLE 63

### 2-{4-[3-(ethoxyimino)-3-(3-methylphenyl)propyl]piperazin-1-yl}nicotinonitrile

### EXAMPLE 63A

### 2-{4-[3-(3-methylphenyl)-3-oxopropyl]piperazin-1-yl}nicotinonitrile

The title compound was prepared using the procedure described in Example 1A except using 3-methylacetophenone and 2-piperazin-1-ylnicotinonitrile instead of 3-chloroacetophenone and 1-(2-pyridinyl)piperazine. MS (DCI/NH₃) m/z 335 (M+H)⁺.

### EXAMPLE 63B

### 2-{4-[3-(hydroxyimino)-3-(3-methylphenyl)propyl]piperazin-1-yl}nicotinonitrile

The title compound was prepared using the procedure described in Example 50C except using the product from example 63A instead of the product from Example 50B. MS (DCI/NH₃) m/z 350 (M+H)⁺.

### EXAMPLE 63C

### 2-{4-[3-(ethoxyimino)-3-(3-methylphenyl)propyl]piperazin-1-yl}nicotinonitrile

The title compound was prepared using the procedure described in Example 51C except using the product from Example 63B instead of the product from Example 51B. maleate salt, mp ° C. ¹H NMR (300 MHz, CDCl₃) δ 1.35 (t, J=7.5 Hz, 3H), 2.4 (s, 3H), 2.62 (m, 6H), 3.0 (m, 2H), 3.72 (m, 3H), 4.25 (q, J=7.5 Hz, 2H), 6.72 (d-d, J=7.5 Hz, 3 Hz, 1H), 7.2 (m, 1H), 7.15 (m, 1H), 7.5 (m, 2H), (d-d, J=7.5 Hz, 3 Hz, 1H), 8.35 (d-d, J=6 Hz, 3 Hz, 1H); MS (DCI/NH₃) m/z 378 (M+H)⁺. Anal. calcd for C₂₆H₃₁N₅O₅; C,63.27; H, 6.33; N, 14.19. Found: C, 63.68; H, 6.17; N, 13.81.

### EXAMPLE 65

### 1-(4-fluorophenyl)-3-[4-(1,3-thiazol-2-yl)-3,6-dihydropyridin-1(2H)-yl]propan-1-one O-methyloxime

### EXAMPLE 65A

### tert-butyl 4-{[(trifluoromethyl)sulfonyl]oxy}-3,6-dihydropyridine-1(2H)-carboxylate

Diisopropylamine (7.74 mL, 55.20 mmol) in THF (100 mL) at −10° C. was treated with n-BuLi (22 mL, 2.5M soln, 55.20 mmol) and stirred for 30 minutes. The mixture was cooled to −78° C. and treated with tert-butyl 4-oxo-1-piperidinecarboxylate (10 g, 50.19 mmol) in THF (50 mL). The mixture was stirred for 15 minutes and treated with N-phenyl-bis-trifluoromethnaesulfonamide (17.8 g, 55.2 mmol) in THF (30 mL) slowly. The mixture was allowed to warm to room temperature, treated with saturated NaHCO₃ and diethyl ether. The organic layer was separated, washed with brine, dried with anhydrous Mg SO₄, filtered, and the filtrate was concentrated under reduced pressure. The residue was purified by chromatography to provide the title compound. MS (DCI/NH₃) m/z 333 (M+H)⁺.

### EXAMPLE 65B

### tert-butyl 4-(1,3-thiazol-2-yl)-3,6-dihydropyridine-1(2H)-carboxylate

2-Thiazolyl zinc bromide (0.5 M solution in THF), (16 mL, 8 mmol), the product from Example 65A (3 g, 9 mmol), and tetrakis(triphenylphosphine) palladium (0) (10% mole, 1 g) were combined in THF and heated at 50° C. for 12 hours. The mixture was allowed to cool to room temperature and treated with brine and ethyl acetate. The acetate layer was separated, dried over anhydrous MgSO₄, filtered, and the filtrate was concentrated under reduced pressure. The residue was purified by chromatography (ethyl acetate:hexane 1:3) to afford the title compound. MS (DCI/NH₃) m/z 267 (M+H)⁺.

### EXAMPLE 65C

### 4-(1,3-thiazol-2-yl)-1,2,3,6-tetrahydropyridine

The product from Example 65B (3.62 g, 13.6 mmol) was treated with a 25% solution of TFA in dichloromethane (30 mL) and stirred for 2 hours. The mixture was then concentrated under reduced pressure to afford the title compound. MS (DCI/NH₃) m/z 167 (M+H)⁺.

### EXAMPLE 65D

### 1-(4-fluorophenyl)-3-[4-(1,3-thiazol-2-yl)-3,6-dihydropyridin-1(2H)-yl]propan-1-one

The title compound was prepared using the procedure described in example 64D except using the product from Example 65C instead of the product from Example 64C to provide the desired ketone. MS (DCI/NH₃) m/z 317 (M+H)⁺.

### EXAMPLE 65E

### 1-(4-fluorophenyl)-3-[4-(1,3-thiazol-2-yl)-3,6-dihydropyridin-1(2H)-yl]propan-1-one O-methyloxime

The title compound was prepared using the procedure described in Example 64E except using the product from Example 65D instead of product from Example 64D. maleate salt, mp ° C. ¹H NMR (300 MHz, DMSO-d6) δ 2.65 (t, J=5 Hz, 2H), 2.90 (m, 3H), 3.18 (m, 2H), 3.5 (m, 1H), 3.90 (m, 5H), 6.55 (m, 1H), 7.15 (m, 2H), 7.6 (d, J=3 Hz, 1H), 7.7 (m, 2H), 7.75 (d, J+3 Hz, 1H); MS (DCI/NH₃) m/z 344 (M+H)⁺. MS (DCI/NH₃) m/z 346 (M+H)⁺. Anal. calcd for C₁₈H₂₀FN₃OS.1.4C₄H₄O₄.0.4H₂O; C,56.29; H, 5.23; N, 8.73; Found: C, 55.97; H, 4.94; N, 9.12.

### EXAMPLE 66

### (1E)-1-(4-chlorophenyl)-2-[4-(1,3-thiazol-2-yl)-3,6-dihydropyridin-1(2H)-yl]ethanone O-methyloxime and

### (1Z)-1-(4-chlorophenyl)-2-[4-(1,3-thiazol-2-yl)-3,6-dihydropyridin-1(2H)-yl]ethanone O-methyloxime

### EXAMPLE 66A

### 1-(4-chlorophenyl)-2-[4-(1,3-thiazol-2-yl)-3,6-dihydropyridin-1(2H)-yl]ethanon

4-Chlorophenacyl chloride (413.51 mg, 2.19 mmol), the product from Example 65C, and K₂CO₃ (1.21 g, 8.76 mmol) were combined in DMF (15 mL) and stirred at room temp for 6 hours. The mixture was partitioned between water and ethyl acetate. The organic extract was separated, washed with water, brine, dried over anhydrous MgSO₄, filtered, and the filtrate was concentrated under reduced pressure. The residue was chromatographed (silica gel, 50% ethyl acetate/hexane) to afford the title compound. MS (DCI/NH₃) m/z 319 (M+H)⁺.

### EXAMPLE 66B

### (1E)-1-(4-chlorophenyl)-2-[4-(1,3-thiazol-2-yl)-3,6-dihydropyridin-1(2H)-yl]ethanone O-methyloxime and

### (1Z)-1-(4-chlorophenyl)-2-[4-(1,3-thiazol-2-yl)-3,6-dihydropyridin-1(2H)-yl]ethanone O-methyloxime

The title compounds were prepared using the procedure described in Example 64E except using the product from Example 66A instead of the product from Example 64D.

(E-isomer): ¹H NMR (300 MHz, DMSO-d6) δ, 2.63 (t,J=5 Hz, 2H), 3.15 (m, 2H), 3.76 (s, 2H), 3.94 (s, 3H), 6.55 (m, 1H), 7.42 (m, 2H), 7.60 (d, J=3 Hz, 1H), 7.8 (m, 3H); MS (DCI/NH₃) m/z 348 (M+H)⁺.

(Z-isomer): maleate salt: mp ° C. ¹H NMR (300 MHz, DMSO-d6) δ, 2.62(m, 2H), 3.15 (m, 2H), 3.40 (m, 2H), 3.80 (s, 3H), 7.50 (m, 3H), 7.60 (m, 2H), 7.8 (d, J=3 Hz, 1H); MS (DCI/NH₃) m/z 348 (M+H)⁺. Anal. calcd for C₁₇H₁₈N₃ClOS.2.8C₄H₄O₄; C,50.34; H, 4.37; N, 6.24. Found: C, 50.49; H, 4.40; N, 5.94.

### EXAMPLE 67

### (1E)-1-(4-fluorophenyl)-3-(3-pyrazin-2-ylpyrrolidin-1-yl)propan-1-one O-methyloxime and

### (1Z)-1-(4-fluorophenyl)-3-(3-pyrazin-2-ylpyrrolidin-1-yl)propan-1-one O-methyloxime

### EXAMPLE 67A

### 2-(1-benzylpyrrolidin-3-yl)pyrazine

2-Vinylpyrazine (10 g, 94.30 mmol) and N-(methoxymethyl)-N-(trimethylsilylmethyl)benzylamine (18.63 g, 79 mmol) were combined in trifluoroacetic acid (0.5 mL) and stirred at room temperature for 12 hours. The mixture was concentrated under reduced pressure and the residue was purified by column chromatography (silica gel, CH₂CL₂ to 10% MeOH/CH₂Cl₂) to afford the title compound. ¹H NMR (300 MHz, CDCl₃) δ 2.10 (m, 1H), 2.36 (m, 1H), 2.74 (m, 3H), 3.04 (t, J=9 Hz, 1H), 3.52 (m, 1H), 3.75 (d, J=3 Hz, 2H), 7.15 (m, 5H), 8.39 (d, J=3 Hz, 1H), 8.46 (m, 1H), 8.57(d, J=2 Hz, 1H); MS (DCI/NH₃) m/z 240 (M+H)⁺.

### EXAMPLE 67B

### 2-pyrrolidin-3-ylpyrazine

The product from Example 67A (2.0 g, 8.40 mmol) was treated with 10% Pd/C (0.4 g), under a 60 psi hydrogen atmosphere at 60° C. for 12 hours. The mixture was cooled, filtered, and the filtrate was concentrated under reduced pressure. The residue was purified by column chromatography (silica gel, 10% MeOH/CH₂Cl₂ to 10% MeOH/CH₂Cl₂/1% NH₄OH) to afford the title compound. ¹H NMR (300 MHz, CDCl₃) δ 2.13 (m, 1H), 2.40 (m, 1H), 2.80 (m, 3H), 3.20 (t, J=9 Hz, 1H), 3.59 (m, 1H), 8.40 (d, J=3 Hz, 1H), 8.46 (m, 1H), 8.52 (d, J=2 Hz, 1H); MS (DCI/NH₃) m/z 150 (M+H)⁺

### EXAMPLE 67C

### 1-(4-fluorophenyl)-3-(3-pyrazin-2-ylpyrrolidin-1-yl)propan-1-one

The product from Example 67B (0.40 g, 2.7 mmol), sodium iodide (0.4 g, 2.7 mmol), cesium chloride (0.97 g, 3.0 mmol), and 3-chloro-4′-fluoropropiophenone (0.5 g, 2.7 mmol) were combined in DMF (20 mL) and stirred at room temperature for 24 hours. The mixture was poured into ethyl acetate (100 mL) and washed with brine, dried over MgSO₄, filtered, and the filtrate was concentrated under reduced pressure. The residue was purified by column chromatography (silica gel, CH₂CL₂ to 5% MeOH/CH₂Cl₂) to afford the title compound. ¹H NMR (300 MHz, CDCl₃) δ 2.12 (m, 1H), 2.36 (m, 1H), 3.06 (m, 8H), 3.60 (m, 1H), 3.58 (m, 1H), 7.15 (t, J=12 Hz, 2H), 8.00 (m, 2H), 8.42 (d, J=3 Hz, 1H), 8.54 (m, 2H); MS (DCI/NH₃) m/z 300 (M+H)⁺.

### EXAMPLE 67D

### (1E)-1-(4-fluorophenyl)-3-(3-pyrazin-2-ylpyrrolidin-1-yl)propan-1-one O-methyloxime and

### (1Z)-1-(4-fluorophenyl)-3-(3-pyrazin-2-ylpyrrolidin-1-yl)propan-1-one O-methyloxime

The product from Example 67C (0.30 g, 1 mmol) and O-methylhydroxylamine hydrochloride (0.42 g, 5 mmol) were combined in pyridine (10 mL) at 0° C. The mixture was allowed to warm to room temperature and stir overnight. The mixture was concentrated under reduced pressure and partitioned between ethyl acetate (100 mL) and saturated NaHCO₃. The organics were separated, dried over MgSO₄, filtered, and the filtrate was concentrated under reduced pressure. The residue was purified by column chromatography (silica gel, ethyl acetate: hexane 1:1) to afford the title compounds.

E Isomer: ¹H NMR (300 MHz, CD₃OD) δ 2.12 (m, 1H), 2.39 (m, 1H), 2.74 (m, 4H), 2.94 (m, 1H), 3.05 (t, J=9 Hz, 2H), 3.18 (t, J=9.5 Hz, 1H), 3.60 (m, 1H), 3.95 (s, 3H), 7.10 (t, J=12 Hz, 2H), 7.78 (m, 2H), 8.42 (d, J=3 Hz, 1H), 8.54 (m, 2H); MS (DCI/NH₃) m/z 329 (M+H)⁺. Anal. calcd for C₁₈H₂₁N₄OF: C, 65.84; H, 6.45; N, 17.06. Found: C, 65.54; H, 6.48; N, 16.87.

Z Isomer: ¹H NMR (300 MHz, CD₃OD) δ 2.09 (m, 1H), 2.35 (m, 1H), 2.75 (m, 7H), 3.10 (t, J=9 Hz, 1H), 3.60 (m, 1H), 3.78 (s, 3H), 7.12 (t, J=12 Hz, 2H), 7.46 (m, 2H), 8.42 (d, J=3 Hz, 1H), 8.54 (m, 1H), 8.97 (d, J=2 Hz, 1H); MS (DCI/NH₃) m/z 329 (M+H)⁺. Anal. calcd for C₁₈H₂₁N₄OF: C, 65.84; H, 6.45; N, 17.06. Found: C, 65.61; H, 6.46; N, 16.94.

### EXAMPLE 68

### (1E)-1-(4-fluorophenyl)-2-(3-pyrazin-2-ylpyrrolidin-1-yl)ethanone O-methyloxime and

### (1Z)-1-(4-fluorophenyl)-2-(3-pyrazin-2-ylpyrrolidin-1-yl)ethanone O-methyloxime

### EXAMPLE 68A

### 1-(4-fluorophenyl)-2-(3-pyrazin-2-ylpyrrolidin-1-yl)ethanone

The product from Example 67B (0.68 g, 4.6 mmol), sodium iodide (0.35 g, 2.3 mmol), cesium chloride (0.15 g, 4.6 mmol), and 2-chloro-4′-fluoroacetophenone (0.8 g, 4.6 mmol) were combined in DMF (10 mL) and stirred at room temperature for 24 hours. The mixture was poured into ethyl acetate (100 mL) and washed with brine, dried over MgSO₄, filtered, and the filtrate was concentrated under reduced pressure. The residue was purified by column chromatography (silica gel, CH₂CL₂ to 4% MeOH/CH₂Cl₂) to afford the title compound. ¹H NMR (300 MHz, CDCl₃) δ 2.12 (m, 1H), 2.34 (m, 1H), 3.06 (m, 5H), 3.59 (m, 1H), 3.55 (m, 1H), 7.10 (t, J=12 Hz, 2H), 7.96 (m, 2H), 8.40 (d, J=3 Hz, 1H), 8.55 (m, 2H); MS (DCI/NH₃) m/z 286 (M+H)⁺.

### EXAMPLE 68B

### (1E)-1-(4-fluorophenyl)-2-(3-pyrazin-2-ylpyrrolidin-1-yl)ethanone O-methyloxime and

### (1Z)-1-(4-fluorophenyl)-2-(3-pyrazin-2-ylpyrrolidin-1-yl)ethanone O-methyloxime

The product from Example 68A (0.24 g, 0.8 mmol) and O-methylhydroxylamine hydrochloride (0.40 g, 4 mmol) were combined in pyridine (10 mL) at 0° C. The mixture was allowed to warm to room temperature and stir overnight. The mixture was concentrated under reduced pressure and partitioned between ethyl acetate (75 mL) and saturated NaHCO₃. The organics were dried over MgSO₄, filtered, and the filtrate was concentrated under reduced pressure. The residue was purified by column chromatography (silica gel, 4% MeOH:CH₂Cl₂) to afford the title compounds.

E Isomer: ¹H NMR (300 MHz, CD₃OD) δ 2.00 (m, 1H), 2.20 (m, 1H), 2.78 (m, 3H), 3.05 (t, J=9 Hz, 1H), 3.53 (m, 1H), 3.84 (s, 2H), 3.95 (s, 3H), 7.10 (t, J=12 Hz, 2H), 7.80 (m, 2H), 8.39 (d, J=3 Hz, 1H), 8.48 (d, J=2 Hz, 1H), 8.54 (m, 1H); MS (DCI/NH₃) m/z 315 (M+H)⁺. Anal. calcd for C₁₇H₁₉N₄OF: C, 64.95; H, 6.09; N, 17.82. Found: C, 64.87; H, 6.18; N, 17.93.

Z Isomer: ¹H NMR (300 MHz, CD₃OD) δ 1.99 (m, 1H), 2.21(m, 1H), 2.80 (m, 3H), 3.02 (t, J=9 Hz, 1H), 3.53 (m, 1H), 3.85 (s, 2H), 3.90 (s, 3H), 7.12 (t, J=12 Hz, 2H), 7.92 (m, 2H), 8.39 (d, J=3 Hz, 1H), 8.43 (d, J=2 Hz, 1H), 8.47 (m, 1H); MS (DCI/NH₃) m/z 315 (M+H)⁺. Anal. calcd for C₁₇H₁₉N₄OF: C, 64.95; H, 6.09; N, 17.82. Found: C, 64.72; H, 5.97; N, 17.91.

### EXAMPLE 69

### (1E)-1-(4-fluorophenyl)-3-(3-pyrazin-2-ylpyrrolidin-1-yl)propan-1-one oxime and

### (1Z)-1-(4-fluorophenyl)-3-(3-pyrazin-2-ylpyrrolidin-1-yl)propan-1-one oxime

The product from Example 67C (0.40 g, 1.3 mmol) in 1,4-dioxane (10 mL), methanol (5 mL), and water (5 mL) was treated with hydroxyamine hydrochloride (0.28 g, 4.0 mmol) and sodium acetate trihydrate (0.55 g, 4.0 mmol) and stirred overnight. The mixture was poured into ethyl acetate (100 mL) and the organic layer was separated, washed with brine, dried over MgSO₄, filtered, and the filtrate was concentrated under reduced pressure. The residue was purified by column chromatography (silica gel,CH₂Cl₂ to 10% MeOH/CH₂Cl₂) to afford the title compounds.

E Isomer: ¹H NMR (300 MHz, CDCl₃) δ 2.12 (m, 1H), 2.37 (m, 1H), 2.72 (m, 4H), 3.06 (m, 3H), 3.12 (t, J=9 Hz, 1H), 3.64 (m, 1H), 7.06 (m, 2H), 7.65 (m, 2H), 8.51 (d, J=3 Hz, 1H), 8.52 (m, 2H); MS (DCI/NH₃) m/z 315 (M+H)⁺. Anal. calcd for C₁₇H₁₉N₄OF: C, 64.95; H, 6.09; N, 17.82. Found: C, 64.65; H, 6.00; N, 17.82.

Z Isomer: ¹H NMR (300 MHz, CDCl₃) δ 2.14 (m, 1H), 2.37 (m, 1H), 2.70 (m, 6H), 3.10 (m, 1H), 3.52 (m, 2H), 7.10 (m, 2H), 7.46 (m, 2H), 8.01 (d, J=3 Hz, 1H), 8.47 (m, 1H), 8.58 (d, J=2 Hz, 1H); MS (DCI/NH₃) m/z 315 (M+H)⁺. Anal. calcd for C₁₇H₁₉N₄OF: C, 64.95; H, 6.09; N, 17.82. Found: C, 64.63; H, 5.94; N, 17.83.

### EXAMPLE 70

### (1E)-1-(4-fluorophenyl)-3-{3-[3-(trifluoromethyl)phenyl]pyrrolidin-1-yl}propan-1-one O-methyloxime and

### (1Z)-1-(4-fluorophenyl)-3-{3-[3-(trifluoromethyl)phenyl]pyrrolidin-1-yl}propan-1-one O-methyloxime

### EXAMPLE 70A

### 1-benzyl-3-[3-(trifluoromethyl)phenyl]pyrrolidine

3-Trifluorostyrene (5 g, 29.10 mmol) and N-(methoxymethyl)-N-(trimethylsilylmethyl)benzylamine (7.57 g, 32 mmol) were combined in trifluoroacetic acid (0.5 mL) and stirred at room temperature for 12 hours. The mixture was concentrated under reduced pressure and the residue was purified by column chromatography (silica gel, CH₂CL₂ to 10% MeOH/CH₂Cl₂) to afford the title compound. ¹H NMR (300 MHz, CDCl₃) δ 1.90 (m, 1H), 2.57 (m, 1H), 2.83 (t, J=9 Hz, 1H), 3.04 (t, J=9 Hz, 1H), 3.61 (m, 1H), 3.69 (m, 2H), 4.05 (s, 2H), 7.15 (m, 5H), 7.48 (m, 2H), 7.58 (m, 2H); MS (DCI/NH₃) m/z 305 (M+H)⁺.

### EXAMPLE 70B

### 3-[3-(trifluoromethyl)phenyl]pyrrolidine

The product from Example 70A (0.50 g, 1.50 mmol) was treated with 10% Pd/C (0.2 g) under a 60 psi hydrogen atmosphere at 60° C. for 12 hours. The mixture was cooled, filtered, and the filtrate was concentrated under reduced pressure. The residue was purified by column chromatography (silica gel, 10% MeOH/CH₂Cl₂ to 10% MeOH/CH₂Cl₂+1% NH₄OH ) to afford the title compound. ¹H NMR (300 MHz, CDCl₃) δ; 1.88 (m, 1H), 2.30 (m, 1H), 2.69 (m, 1H), 2.90 (t, J=9 Hz, 1H), 3.26 (m, 3H), 7.42 (m, 4H); MS (DCI/NH₃) m/z 216 (M+H)⁺.

### EXAMPLE 70C

### 1-(4-fluorophenyl)-3-{3-[3-(trifluoromethyl)phenyl]pyrrolidin-1-yl}propan-1-one

The product from Example 70B (0.70 g, 3.30 mmol), sodium iodide (0.03 g, 2.0 mmol), cesium chloride (1.16 g, 3.6 mmol), and 3-Chloro-4′-fluoropropiophenone (0.61 g, 3.3 mmol) were combined in DMF (10 mL) and stirred at room temperature for 16 hours. The mixture was poured into ethyl acetate (100 mL) and washed with brine, dried over MgSO₄, filtered, and the filtrate was concentrated under reduced pressure. The residue was purified by column chromatography (silica gel, CH₂CL₂ to 10% MeOH/CH₂Cl₂) to afford the title compound. ¹H NMR (300 MHz, CDCl₃) δ 1.87 (m, 1H), 2.36 (m, 1H), 2.62 (t, J=9 Hz, 1H), 2.81 (t, J=9 Hz, 1H), 3.09 (m, 6H), 3.36 (m, 1H), 7.15 (t, J=12 Hz, 2H), 7.39 (m, 3H), 7.99 (m, 3H); MS (DCI/NH₃) m/z 366 (M+H)⁺.

### EXAMPLE 70D

### (1E)-1-(4-fluorophenyl)-3-{3-[3-(trifluoromethyl)phenyl]pyrrolidin-1-yl}propan-1-one O-methyloxime and

### (1Z)-1-(4-fluorophenyl-3-{3-[3-(trifluoromethyl)phenyl]pyrrolidin-1-yl}propan-1-one O-methyloxime

The product from Example 70C (0.20 g, 0.5 mmol) and O-methylhydroxylamine hydrochloride (0.23 mg, 2.7 mmol) were combined in pyridine (10 mL) at 0° C. and allowed to warm to room temperature overnight. The mixture was concentrated under reduced pressure and the residue was partitioned between ethyl acetate and saturated NaHCO₃. The organics were dried over MgSO₄, filtered, and the filtrate was concentrated under reduced pressure. The residue was purified by column chromatography (silica gel, CH₂Cl₂ to 4% MeOH/CH₂Cl₂) to afford the title compounds.

E Isomer: ¹H NMR (300 MHz, CD₃OD) δ 1.88 (m, 1H), 2.35 (m, 1H), 2.72 (m, 7H), 3.00 (m, 1H), 3.45 (m, 1H), 3.79 (s, 3H), 7.07 (m, 2H), 7.46 (m, 3H), 7.65 (m, 2H), 7.76 (m, 1H); MS (DCI/NH₃) m/z 395 (M+H)⁺. Anal. calcd for C₂₁H₂₂N₂OF₄: C, 63.46; H, 5.68; N, 6.52. Found: C, 63.95; H, 5.62; N, 7.10.

Z Isomer: ¹H NMR (300 MHz, CD₃OD) δ 1.86 (m, 1H), 2.36 (m, 1H), 2.64 (m, 5H), 3.07 (m, 3H), 3.42 (m, 1H), 3.96 (s, 3H), 7.10 (m, 2H), 7.45 (m, 2H), 7.51 (m, 2H), 7.60 (m, 2H); MS (DCI/NH₃) m/z 395 (M+H)⁺. Anal. calcd for C₂₁H₂₂N₂OF₄: C, 63.46; H, 5.68; N, 6.52. Found: C, 63.76; H, 5.68; N, 6.79.

### EXAMPLE 71

### (1Z)-1-(4-fluorophenyl)-2-(3-pyrazin-2-ylpyrrolidin-1-yl)ethanone oxime

The product from Example 70C (0.97 g, 3.4 mmol), hydroxylamine hydrochloride (0.71 g, 10.20 mmol), and sodium acetate trihydrate (1.39 g, 10.20 mmol) were combined in 1,4-dioxane (10 mL), methanol (5 mL), and water (5 mL) and stirred overnight. The mixture was then poured into ethyl acetate (100 mL) and the acetate layer was washed with brine, dried over MgSO₄, filtered, and the filtrate was concentrated under reduced pressure. The residue was purified by column chromatography (silica gel,CH₂Cl₂ to 10% MeOH/CH₂Cl₂) to afford the title compound.

E and Z Isomer mixture: ¹H NMR (300 MHz, CD₃OD) δ 1.88 (m, 1H), 2.35 (m, 1H), 2.75 (m, 3H), 3.10 (m, 1H), 3.47 (m, 1H), 3.59 (s, 2H), 7.10 (m, 2H), 7.45 (m, 2H), 7.70 (m, 1H), 7.85 (m, 1H), 8.47 (m, 1H); MS (DCI/NH₃) m/z 301 (M+H)⁺. Anal. calcd for C₁₆H₁₇N₄OF: C, 63.99; H, 5.71; N, 18.65. Found: C, 64.26; H, 5.85; N, 19.01.

### EXAMPLE 72

### 1-(4-fluorophenyl)-3-[3-(2-methoxyphenyl)pyrrolidin-1-yl]propan-1-one O-methyloxime

### EXAMPLE 72A

### 1-methoxy-2-vinylbenzene

1,3-Divinyltetramethyldisiloxane (5.1 g, 27 mmol), potassium trimethylsilanolate (8.8 g, 68 mmol), tris(dibenzylideneacetone)dipalladium(0) (300 mg, 0.3 mmol) and 2-iodoanisole (5.8 g, 25 mmol) were combined in THF (15 mL) and stirred at 45° C. for 18 hours. The mixture was concentrated under reduced pressure and the residue was purified by column chromatography using hexanes as the eluent to provide the title compound. MS (DCI/NH₃) m/z 135 (M+H)⁺.

### EXAMPLE 72B

### 1-benzyl-3-(2-methoxyphenyl)pyrrolidine

The product from Example 72A (1.4 g, 10 mmol) and N-(methoxymethyl)-N-(trimethylsilylmethyl)benzylamine (2.4 g, 10 mmol) were combined in dichloromethane (5 mL), treated with trifluoroacetic acid (12 μL), and heated at 45° C. for 18 hours. The mixture was allowed to cool to room temperature and concentrated under reduced pressure. The residue was purified on silica gel column (hexanes:ethyl acetate 4:1) to afford the title compound. MS (DCI/NH₃) m/z 268 (M+H)⁺.

### EXAMPLE 72C

### 3-(2-methoxyphenyl)pyrrolidine

The product from Example 72B (1 g, 3.8 mmol) was hydrogenated with 20% palladium hydroxide/carbon (0.75 g) in methanol (10 mL) under 60 psi of H₂ for 17 hours. The mixture was filtered and the filtrate was concentrateded under reduced pressure to afford the title compound. MS (DCI/NH₃) m/z 178 (M+H)⁺.

### EXAMPLE 72D

### 1-(4-fluorophenyl)-3-[3-(2-methoxyphenyl)pyrrolidin-1-yl]propan-1-one

The product from Example 72C (210 mg, ˜1.2 mmol), 3-chloro-4′-fluoro-propiophenone (330 mg, 1.7 mmol), potassium carbonate (220 mg, 1.6 mmol), and sodium iodide (240 mg 1.6 mmol) were combined in DMF (2 mL) and stirred at room temperature for 17 hours. The mixture was diluted with dichloromethane (5 mL), washed with water, brine, dried over magnesium sulfate, filtered, and the filtrate was concentrated under reduced pressure to provide the title compound. MS (DCI/NH₃) m/z 328 (M+H)⁺.

### EXAMPLE 72E

### 1-(4-fluorophenyl)-3-[3-(2-methoxyphenyl)pyrrolidin-1-yl]propan-1-one O-methyloxime

The product from Example 72D (100 mg, 0.3 mmol) and O-methylhydroxylamine hydrochloride (125 mg, 1.5 mmol) were combined in pyridine (7 mL) and stirred at room temperature for 17 hours. The mixture was concentrated under reduced pressure and the residue was treated with ethyl acetate. The ethyl acetate was washed with saturated NaHCO₃, dried over MgSO₄, filtered, and the filtrate was concentrated under reduced pressure. The residue was purified by chromatography (gradient of 10% to 100% ethyl acetate in hexane) to provide the title product. ¹H NMR (300 MHz, MeOH-d₄) δ 1.91 (m, 1H), 2.23 (m, 1H), 2.53 (t, J=9 Hz, 1H), 2.69 (m, 3H), 2.92 (m, 1H), 3.02 (t, J=8 Hz, 2H), 3.10 (m, 1H), 3.70 (m, 1H), 3.81 (s, 3H), 3.96 (s, 3H), 6.90 (m, 2H), 7.12 (m, 2H), 7.21 (m, 2H), 7.71 (m, 2H); MS (DCI/NH₃) m/z 357 (M+H)⁺.

### EXAMPLE 73

### (1E)-1-(4-fluorophenyl)-3-[3-(3-methoxyphenyl)pyrrolidin-1-yl]propan-1-one O-methyloxime and

### (1Z)-1-(4-fluorophenyl)-3-[3-(3-methoxyphenyl)pyrrolidin-1-yl]propan-1-one O-methyloxime

### EXAMPLE 73A

### 1-methoxy-3-vinylbenzene

The title compound was prepared using the procedure described in Example 72A except using 3-iodoanisole instead of 2-iodoanisole.

### EXAMPLE 73B

### 1-benzyl-3-(3-methoxyphenyl)pyrrolidine

The title compound was prepared using the procedure described in Example 72B except using the product from Example 73A instead of the product from Example 72A.

### EXAMPLE 73C

### 3-(3-methoxyphenyl)pyrrolidine

The title compound was prepared using the procedure described in Example 72C except using the product from Example 73B instead of the product from Example 72B.

### EXAMPLE 73D

### 1-(4-fluorophenyl)-3-[3-(3-methoxyphenyl)pyrrolidin-1-yl]propan-1-one

The title compound was prepared using the procedure described in Example 72D except using the product from Example 73C instead of the product from Example 72C.

### EXAMPLE 73E

### 1E)-1-(4-fluorophenyl)-3-[3-(3-methoxyphenyl)pyrrolidin-1-yl]propan-1-one O-methyloxime and

### (1Z)-1-(4-fluorophenyl)-3-[3-(3-methoxyphenyl)pyrrolidin-1-yl]propan-1-one O-methyloxime

The title compounds were prepared using the procedure described in Example 72E except using the product from Example 73D instead of the product from Example 72D.

E-isomer: ¹H NMR (300 MHz, MeOH-d₄) δ 1.89 (m, 1H), 2.29 (m,1H), 2.57 (dd, J=9.5 Hz, 8.5 Hz, 1H), 2.70 (m, 3H), 2.89 (m, 1H), 3.02 (t, J=8 Hz, 2H), 3.09 (m, 1H), 3.36 (m, 1H), 3.77 (s, 3H), 3.95 (s, 3H), 6.74 (m, 1H), 6.83 (m, 2H), 7.11 (m, 2H), 7.18 (m, 1H), 7.71 (m, 2H); MS (DCI/NH₃) m/z 357 (M+H)⁺.

Z-isomer: ¹H NMR (300 MHz, MeOH-d₄) δ 1.86 (m, 1H), 2.27 (m, 1H), 2.55 (dd, J=9.5 Hz, 8 Hz, 1H), 2.67 (m, 2H), 2.80 (m, 3H), 3.01 (m, 1H), 3.33 (m, 1H), 3.77 (s, 3H), 3.78 (s, 3H), 6.74 (m, 1H), 6.83 (m, 2H), 7.12 (m, 2H), 7.18 (m, 1H), 7.48 (m, 2H); MS (DCI/NH₃) m/z 357 (M+H)⁺.

### EXAMPLE 74

### (1E)-1-(4-fluorophenyl)-3-[3-(4-methoxyphenyl)pyrrolidin-1-yl]propan-1-one O-methyloxime and

### (1Z)-1-(4-fluorophenyl)-3-[3-(4-methoxyphenyl)pyrrolidin-1-yl]propan-1-one O-methyloxime

### EXAMPLE 74A

### 1-methoxy4-vinylbenzene

The title compound was prepared using the procedure described in Example 72A except using 4-iodoanisole instead of 2-iodoanisole.

### EXAMPLE 74B

### 1-benzyl-3-(4-methoxyphenyl)pyrrolidine

The title compound was prepared using the procedure described in Example 72B except using the product from Example 74A instead of the product from Example 72A.

### EXAMPLE 74C

### 3-(4-methoxyphenyl)pyrrolidine

The title compound was prepared using the procedure described in Example 72C except using the product from Example 74B instead of the product from Example 72B.

### EXAMPLE 74D

### 1-(4-fluorophenyl)-3-[3-(4-methoxyphenyl)pyrrolidin-1-yl]propan-1-one

The title compound was prepared using the procedure described in Example 72D except using the product from Example 74C instead of the product from Example 72C.

### EXAMPLE 74E

### 1E)-1-(4-fluorophenyl)-3-[3-(4-methoxyphenyl)pyrrolidin-1-yl]propan-1-one O-methyloxime and

### (1Z)-1-(4-fluorophenyl)-3-[3-(4-methoxyphenyl)pyrrolidin-1-yl]propan-1-one O-methyloxime

The title compounds were prepared using the procedure described in Example 72E except using the product from Example 74D instead of the product from Example 72D.

E-isomer: ¹H NMR (300 MHz, MeOH-d₄) δ 1.85 (m, 1H), 2.27 (m, 1H), 2.50 (t, J=9 Hz, 1H,) 2.67 (m, 3H), 2.91 (m, 1H), 3.01 (m, 2H), 3.09 (m, 1H), 3.31 (m, 1H), 3.76 (s, 3H), 3.95 (s, 3H,) 6.83 (d, J=9 Hz, 2H,) 7.10 (d, J=9 Hz, 2H), 7.16 (d, J=9 Hz, 2H), 7.70 (m, 2H); MS (DCI/NH₃) m/z 357 (M+H)⁺.

Z-isomer: ¹H NMR (300 MHz, MeOH-d₄) δ 1.83 (m, 1H,) 2.26 (m, 1H), 2.48 (t, J=9 Hz, 1H), 2.63 (m, 3H,) 2.75 (m, 2H), 2.83 (m, 1H), 3.03 (m, 1H), 3.29 (m, 1H), 3.75 (s, 3H), 3.78 (s, 3H), 6.83 (d, J=9 Hz, 2H), 7.11 (d, J=9 Hz, 2H), 7.17 (m, 2H), 7.48 (m, 2H); MS (DCI/NH₃) m/z 357 (M+H)⁺.

### EXAMPLE 75

### 1-(4-fluorophenyl)-3-[3-(1,3-thiazol-2-yl)piperidin-1-yl]propan-1-one O-methyloxime

### EXAMPLE 75A

### tert-butyl 5-{[(trifluoromethyl)sulfonyl]oxy}-3,6-dihydropyridine-1(2H)-carboxylate

Diisopropylamine (13.1 mL, 110 mmol) in THF (150 mL) at −10° C. was treated with n-BuLi (2.5 M soln in hexane, 44 mL, 110 mmol) and stirred for 30 minutes at −10° C. The mixture was cooled to −78° C. and treated with a solution of tert-butyl-3-oxo-1-piperidinecarboxylate (16 g, 80 mmol) in THF (50 mL). The mixture was stirred for 15 minutes and treated with a solution of N-phenyl-bis-trifluoromethnaesulfonamide (35.0 g, 110 mmol) in THF (60 mL). The mixture was allowed to warm to ambient temperature, treated with saturated NaHCO₃ (75 mL), and extracted with diethyl ether. The ether extract was washed with brine, dried over anhydrous MgSO₄, filtered, and the filtrate was concentrated under reduced pressure. The residue was purified by flash chromatography (silica gel, ethyl acetate:hexanes 0.5:9.5) to provide the title compound. MS (DCI/NH₃) m/z 333 (M+H)⁺.

### EXAMPLE 75B

### tert-butyl 5-(1,3-thiazol-2-yl)-3,6-dihydropyridine-1(2H)-carboxylate

The 2-thiazolyl zinc bromide (20 mL, 10 mmol, 0.5 M solution in THF), the product from Example 75A (3.3 g, 10 mmol), and tetrakis(tripehynlphosphine) Pd(0) (10% mole, 1.1 g) were combined in dry THF (30 mL) at 0° C. The mixture was heated at 50° C. for 1 hour, cooled to room temperature, and treated with brine. The mixture was extracted with ethyl acetate and the acetate layer was dried over anhydrous MgSO₄, filtered, and the filtrate was concentrated under reduced pressure. The residue was purified by flash chromatography (silica gel, ethyl acetate:hexanes 1:3) to afford the title compound. MS (DCI/NH₃) m/z 265 (M+H)⁺.

### EXAMPLE 75C

### tert-butyl 3-(1,3-thiazol-2-yl)piperidine-1-carboxylate

The product from Example 75B (1.4 g, 5.3 mmol) was treated with 20% Pd/C (0.7 g) in methanol (50 mL) under a hydrogen atmosphere for 4 days at room temperature. The mixture was filtered and the filtrate was concentrated under reduced pressure to provide the title compound. MS (DCI/NH₃) m/z 267 (M+H)⁺.

### EXAMPLE 75D

### 3-(1,3-thiazol-2-yl)piperidine

The product from Example 75C (1.2 g, 4.5 mmol) was treated with 25% TFA in dichloromethane (10 mL). After 2 hours, the mixture was concentrated under reduced pressure to provide the title compound which was used directly in next step without further purification.

### EXAMPLE 75E

### 1-(4-fluorophenyl)-3-[3-(1,3-thiazol-2-yl)piperidin-1-yl]propan-1-one

3-Chloro-4′fluoropropiophenone (0.465 g, 2.5 mmol), the product from Example 75D (0.42 g, 2.5 mmol), K₂CO₃ (0.348 g, 2.5 mmol), and NaI (0.37 g, 2.5 mmol) and were combined in DMF(5 mL) and stirred at room temperature for 16 hours. The mixture was diluted with ethyl acetate (30 mL) and washed with brine. The organic layer was dried with MgSO₄, filtered, and the filtrate was concentrated under reduced pressure. The residue was purified by flash column chromatography (silica gel, 1:9 ethanol:ethyl acetate) to provide the title compound.

### EXAMPLE 75F

### 1-(4-fluorophenyl)-3-[3-(1,3-thiazol-2-yl)piperidin-1-yl]propan-1-one O-methyloxime

The product from Example 75E (150 mg, 0.5 mmol) and O-methylhydroxylamine hydrochloride (200 mg, 2.5 mmol) were combined in pyridine (10 mL) and stirred at ambient temperature for 12 hours, The mixture was concentrated under reduced pressure and partitioned between saturated NaHCO₃ and ethyl acetate. The acetate extract was separated, washed with brine, dried with MgSO₄, filtered, and the filtrate was concentrated under reduced pressure. The residue was purified by flash column chromatography (silica gel, 1:9 ethanol:ethyl acetate) to provide the title compound. ¹H NMR (300 MHz, DMSO-d₆) δ 1.51 (m, 2H), 1.68 (m, 1H), 1.98 (m, 1H), 2.08 (m, 2H), 2.22 (t, J=12 Hz, 1H), 2.73 (m, 1H), 2.91 (m, 2H), 3.11 (m, 3H), 3.93 (s, 3H), 7.23 (t, J=9 Hz, 2H), 7.58 (d, J=3 Hz, 1H), 7.67 (t, J=4.5 Hz, 2H), 7.71(d, J=3 Hz, 1H); MS (DCI/NH₃) m/z 348 (M+H)⁺. Anal. calcd for C₁₈H₂₂FN₃OS: C, 62.22; H, 6.38; N, 12.09. Found: C, 61.91; H, 6.38; N, 11.98.

### EXAMPLE 76

### 1-(4-fluorophenyl)-3-[3-(1,3-thiazol-2-yl)piperidin-1-yl]propan-1-one O-ethyloxime

### EXAMPLE 76A

### 1-(4-fluorophenyl)-3-[3-(1,3-thiazol-2-yl)piperidin-1-yl]propan-1-one oxime

The product from Example 75E (0.4 g, 1.3 mmol), hydroxylamine hydrochloride (0.45 g, 6.5 mmol) and sodium acetate trihydrate (0.884 g, 6.5 mmol) were combined in ethanol:H₂O (8:2, 20 mL) and stirred at room temperature for 12 hours. The mixture was diluted with ethyl acetate (30 mL), washed with brine, dried over MgSO₄, filtered, and the filtrate was concentrated under reduced pressure. The residue was purified by flash column chromatography (silica gel, 1:9 ethanol:ethyl acetate) to afford the title compound.

### EXAMPLE 76B

### 1-(4-fluorophenyl)-3-[3-(1,3-thiazol-2-yl)piperidin-1-yl]propan-1-one O-ethyloxime

The product from Example 76A (0.16 g, 0.5 mmol) and potassium tert-butoxide.(0.062 g, 0.55 mmol) were combined in t-butanol (10 mL) and refluxed for 30 minutes under N₂. The mixture was cooled to 50° C. and treated with ethyl iodide (2 mL). The mixture was refluxed for 1 hour, allowed to cool to room temperature, and concentrated under reduced pressure. The residue was diluted with ethyl acetate (30 mL), washed with brine, dried with MgSO₄, filtered, and the filtrate was concentrated under reduced pressure. The residue was purified by flash column chromatography (silica gel, 1:9 ethanol:ethyl acetate) to provide the title compound. ¹H NMR (300 MHz, CDCl₃) δ 1.10 (t, J=6 Hz, 3H), 1.61 (m, 2H), 1.78 (m, 1H), 2.18 (m, 2H), 2.35 (t, J=12 Hz, 1H), 2.63 (m, 2H), 2.95 (m, 3H), 3.28 (m, 2H), 4.22 (q, J=6 Hz, 2H), 7.04 (t, J=9 Hz, 2H), 7.21 (d, J=3 Hz, 1H), 7.63 (d-d, J=6 Hz, 3 Hz, 2H), 7.72 (d, J=3 Hz, 1H); MS (DCI/NH₃) m/z 362 (M+H)⁺. Anal. calcd for C₁₉H₂₄FN₃OS: C, 63.02; H, 6.69; N, 11.62 Found: C, 62.77; H, 6.50; N, 11.39.

### EXAMPLE 77

### 1-(4-fluorophenyl)-3-(3-pyridin-2-ylpiperidin-1-yl)propan-1-one O-methyloxime

### EXAMPLE 77A

### tert-butyl 5′,6′-dihydro-2,3′-bipyridine-1′(2′H)-carboxylate

The title compound was prepared using the procedure described in Example 75B except using 2-pyridinyl-zinc bromide instead of 2-thiazolyl zinc bromide.

### EXAMPLE 77B

### tert-butyl 3-pyridin-2-ylpiperidine-1-carboxylate

The title compound was prepared using the procedure described in Example 75C except using the product from Example 77A instead of the product from Example 75B.

### EXAMPLE 77C

### 2-piperidin-3-ylpyridine

The title compound was prepared using the procedure described in Example 75D except using the product from Example 77B instead of the product from Example 75C.

### EXAMPLE 77D

### 1-(4-fluorophenyl)-3-(3-pyridin-2-ylpiperidin-1-yl)propan-1-one

The title compound was prepared using the procedure described in Example 75E except using the product from Example 77C instead of the product from Example 75D.

### EXAMPLE 77E

### 1-(4-fluorophenyl)-3-(3-pyridin-2-ylpiperidin-1-yl)propan-1-one O-methyloxime

The title compound was prepared using the procedure described in Example 75F except using the product from Example 77D instead of the product from Example 75E. ¹H NMR (300 MHz, CDCl₃) δ 1.61 (m, 2H), 1.82 (m, 1H), 1.98 (m, 2H), 2.05 (m, 1H), 2.43 (m, 2H), 2.82 (m, 3H), 3.12 (m, 2H), 3.81 (s, 3H), 7.13 (m, 2H), 7.35 (d, J=9 Hz, 2H), 7.58 (d, J=9 Hz, 2H), 7.63 (m, 1H), 8.57 (m, 1H); MS (DCI/NH₃) m/z 342 (M+H)⁺. Anal. calcd for C₂₀H₂₄FN₃O.H2O: C, 66.92; H, 7.14; N, 11.70. Found: C, 67.13; H, 6.91; N, 11.53.

### EXAMPLE 78

### 1-(4-chlorophenyl)-3-(3-pyridin-2-ylpiperidin-1-yl)propan-1-one O-methyloxime

### EXAMPLE 78A

### 1-(4-chlorophenyl)-3-(3-pyridin-2-ylpiperidin-1-yl)propan-1-one

The title compound was prepared using the procedure described in Example 75E except using the product from Example 77C and 3,4′-dichloropropiophenone instead of 3-chloro-4′fluoropropiophenone and the product from Example 75D.

### EXAMPLE 78B

### 1-(4-chlorophenyl)-3-(3-pyridin-2-ylpiperidin-1-yl)propan-1-one O-methyloxime

The title compound was prepared using the procedure described in Example 75F except using the product from Example 78A instead of the product from Example 75E. ¹H NMR (300 MHz, CDCl₃) δ 1.63 (m, 3H), 1.79 (m, 1H), 1.97 (m, 1H), 2.15 (m, 1H), 2.26 (t, J=9 Hz, 1H), 2.59 (m, 2H), 2.95 (m, 3H), 3.12 (m, 1H), 3.97 (s, 3H), 7.13 (m, 2H), 7.35 (d, J=9 Hz, 2H), 7.58 (d, J=9 Hz, 2H), 7.63 (m, 1H), 8.57 (m, 1H), MS (DCI/NH₃) m/z 358 (M+H)⁺. Anal. calcd for C₂₀H₂₄ClN₃O.0.25H2O: C, 66.31; H, 6.71; N, 11.63. Found: C, 66.09; H, 6.80; N, 11.42.

EXAMPLE 79

### (1E)-1-(4-chlorophenyl)-3-[3-(1,3-thiazol-2-yl)piperidin-1-yl]propan-1-one O-methyloxime and

### (1Z)-1-(4-chlorophenyl)-3-[3-(1,3-thiazol-2-yl)piperidin-1-yl]propan-1-one O-methyloxime

### EXAMPLE 79A

### 1-(4-chlorophenyl)-3-[3-(1,3-thiazol-2-yl)piperidin-1-yl]propan-1-one

The title compound was prepared using the procedure described in Example 75E except using 3,4′-dichloropropiophenone instead of the 3-chloro-4′fluoropropiophenone.

### EXAMPLE 79B

### (1E)-1-(4-chlorophenyl)-3-[3-(1,3-thiazol-2-yl)piperidin-1-yl]propan-1-one O-methyloxime and

### (1Z)-1-(4-chlorophenyl)-3-[3-(1,3-thiazol-2-yl)piperidin-1-yl]propan-1-one O-methyloxime

The title compounds were prepared using the procedure described in Example 75F except using the product from Example 79A instead of the product from Example 75E.

E-Isomer: 1.65 (m, 3H), 1.79 (m, 1H), 2.15 (m, 2H), 2.33 (t, J=12 Hz, 1H), 2.59 (m, 1H), 2.83 (m, 1H), 2.95 (m, 2H), 3.25 (m, 2H), 3.98 (s, 3H), 7.21 (d, J=3 Hz, 1H), 7.35 (d, J=9 Hz, 2H), 7.58 (d, J=9 Hz, 2H), 7.71 (d, J=3 Hz, 1H); MS (DCI/NH₃) m/z 364 (M+H)⁺. Anal. calcd for C₁₈H₂₂ClN₃OS: C, 59.41; H, 6.09; N, 11.55. Found: C, 59.09; H, 6.21; N, 11.53.

Z-Isomer: 1.60 (m, 3H), 1.77 (m, 1H), 2.10 (m, 2H), 2.27 (t, J=12 Hz, 1H), 2.52 (m, 1H), 2.73 (m, 1H), 2.81 (m, 2H), 3.10 (m, 1H), 3.25 (m, 1H), 3.81 (s, 3H), 7.11 (d, J=3 Hz, 1H), 7.35 (m, 4H), 7.68 (d, J=3 Hz, 1H); MS (DCI/NH₃) m/z 364 (M+H)⁺.

Anal. calcd for C₁₈H₂₂ClN₃OS: C, 59.41; H, 6.09; N, 11.55. Found: C, 59.18; H, 6.11; N, 11.63.

### EXAMPLE 80

### 1-(4-fluorophenyl)-3-(3-phenylpiperidin-1-yl)propan-1-one O-methyloxime

### EXAMPLE 80A

### tert-butyl 5-phenyl-3,6-dihydropyridine-1(2H)-carboxylate

The title compound was prepared using the procedure described in Example 75B except using phenyl zinc bromide instead of 2-thiazolyl zinc bromide.

### EXAMPLE 80B

### tert-butyl 3-phenylpiperidine-1-carboxylate

The title compound was prepared using the procedure described in Example 75C except using the product from Example 80A instead of the product from Example 75B.

### EXAMPLE 80C

### 3-phenylpiperidine

The title compound was prepared using the procedure described in Example 75D except using the product from Example 80B instead of the product from Example 75C.

### EXAMPLE 80D

### 1-(4-fluorophenyl)-3-(3-phenylpiperidin-1-yl)propan-1-one

The title compound was prepared using the procedure described in Example 75E except using the product from Example 80C instead of the product from Example 75D.

### EXAMPLE 80E

### 1-(4-fluorophenyl)-3-(3-phenylpiperidin-1-yl)propan-1-one O-methyloxime

The title compound was prepared using the procedure described in Example 75F except using the product from Example 80D instead of the product from Example 75E. ¹H NMR (300 MHz, CDCl₃) δ 1.46 (m, 1H), 1.73 (m, 2H), 1.92 (m, 1H), 2.08 (t J=12 Hz, 2H), 2.57 (m, 2H), 2.81 (m, 1H), 2.98 (m, 4H), 3.97 (s, 3H), 7.05 (t, J=9 Hz, 2H), 7.26 (m, 5H), 7.62 (d-d, J=9 Hz, 4.5 Hz, 2H); MS (DCI/NH₃) m/z 341 (M+H)⁺.

Anal. calcd for C₂₁H₂₅FN₂O.0.25H2O: C, 73.15; H, 7.33; N, 8.13. Found: C, 73.30; H, 7.38; N, 8.08.

### EXAMPLE 81

### 1-phenyl-3-(3-phenylpiperidin-1-yl)propan-1-one O-methyloxime

### EXAMPLE 81A

### 1-phenyl-3-(3-phenylpiperidin-1-yl)propan-1-one

The title compound was prepared using the procedure described in Example 75E except using 3-chloro-propiophenone and the product from Example 80C instead of 3-chloro-4′fluoro-propiophenone and the product from Example 75D.

### EXAMPLE 81B

### 1-phenyl-3-(3-phenylpiperidin-1-yl)propan-1-one O-methyloxime

The title compound was prepared using the procedure described in Example 75F except using the product from Example 81A instead of the product from Example 75E. ¹H NMR (300 MHz, CDCl₃) δ 1.50 (m, 1H), 1.78 (m, 2H), 1.92 (m, 1H), 2.08 (t, J=12 Hz, 2H), 2.59 (m, 2H), 2.83 (m, 1H), 2.98 (m, 4H), 3.97 (s, 3H), 7.22 (m, 5H), 7.37 (m, 3H), 7.62 (d-d, J=9 Hz, 6 Hz, 2H); MS (DCI/NH₃) m/z 323 (M+H)⁺.

Anal. calcd for C₂₁H₂₆N₂O.0.25H2O: C, 77.19; H, 8.04; N, 8.58. Found: C, 77.32; H, 8.13; N, 8.60.

### EXAMPLE 82

### 1-(4-chlorophenyl)-3-(3-phenylpiperidin-1-yl)propan-1-one O-methyloxime

### EXAMPLE 82A

### 1-(4-chlorophenyl)-3-(3-phenylpiperidin-1-yl)propan-1-one

The title compound was prepared using the procedure described in Example 75E except using 3,4′-dichloropropiophenone and the product from Example 80C instead of 3-chloro-4′fluoro-propiophenone and the product from Example 75D.

### EXAMPLE 82B

### 1-(4-chlorophenyl)-3-(3-phenylpiperidin-1-yl)propan-1-one O-methyloxime

The title compound was prepared using the procedure described in Example 75F except using the product from Example 82A instead of the product from Example 75E. ¹H NMR (300 MHz, CDCl₃) δ 1.58 (m, 1H), 1.78 (m, 2H), 1.88 (m, 1H), 2.05 (t, J=12 Hz, 2H), 2.56 (m, 2H), 2.82 (m, 1H), 2.96 (m, 4H), 3.97 (s, 3H), 7.30 (m, 7H), 7.59 (d, J=9 Hz, 2H); MS (DCI/NH₃) m/z 357 (M+H)⁺. Anal. calcd for C₂₁H₂₅ClN₂O.0.25H2O: C, 69.78; H, 6.99; N, 7.76. Found: C, 70.03; H, 6.86; N, 7.73.

### EXAMPLE 83

### 2-{1-[2-(4-fluorophenyl)-2-(methoxyimino)ethyl]piperidin-3-ylpyridinium N-oxide

### EXAMPLE 83A

### 2-[1-(tert-butoxycarbonyl)piperidin-3-yl]pyridinium N-oxide

tert-Butyl 3-pyridin-2-ylpiperidine-1-carboxylate (980 mg, 3.7 mmol) in dichloromethane (20 mL) at 0° C. was added dropwise to a solution of meta-chloroperoxybenzoic acid (1.21 g, 7.0 mmol) in dichloromethane (10 mL). The solution was allowed to warm to room temperature and stirred for 2 hours. The mixture was washed with NaHCO₃ and brine, dried with Na₂SO₄, filtered, and the filtrate was concentrated under reduced pressure. The residue was purified by column chromatography (silica gel, 30:1 CH₂Cl₂/methanol) to provide the title compound. ¹H NMR (300 MHz, chloroform-d) δ 1.47 (m, 9H), 1.67 (b, 3H), 2.18 (b, 1H), 3.02 (b, 2H), 3.65 (b, 1H), 3.97 (b, 1H), 4.19 (b, 1H), 7.16 (m, 2H), 7.24 (d, J=6 Hz, 1H), 8.29 (d, J=9 Hz, 1H); MS (APCI) m/z 279 (M+H)⁺.

### EXAMPLE 83B

### 2-piperidin-3-ylpyridinium N-oxide

The product from Example 83A (870 mg, 3.1 mmol) in ethyl acetate (20 mL) was treated with HCl gas at −78° C. for 15 minutes. The mxiture was allowed to warm to room temperature and was concentrated under reduced pressure. Toluene was added and removed under reduced pressure (×2) to provide the title compound which was used in the next step without further purification. MS (APCI) m/z 179 (M+H)⁺.

### EXAMPLE 83C

### 2-{1-[2-(4-fluorophenyl)-2-oxoethyl]piperidin-3-yl}pyridinium N-oxide

The product from Example 83B (160 mg, 0.75 mmol), 2-chloro-4′-fluoroacetophenone (155 mg, 0.90 mmol), K₂CO₃ (291 mg, 2.1 mmol), and NaI (134 mg, 0.9 mmol) were combined in DMF (8 mL) and stirred at 50° C. for 2 hours. The mixture was diluted with H₂O and extracted with ethyl acetate. The organic layers were separated, combined, washed with brine, dried with Na₂SO₄, filtered, and the filtrate was concentrated under reduced pressure. The residue was chromatographed (silica gel, 20:1 CH₂Cl₂/methanol) to afford the title compound. An analytical pure sample was prepared as the maleate salt. ¹H NMR (300 MHz, MeOH-d₄) δ 2.13 (m, 2H), 2.28 (m, 1H), 3.28 (m, 1H), 3.33 (m, 1H), 3.52 (t, J =12 Hz, 1H), 3.80 (m, 1H), 4.05 (m, 1H), 4.35(m, 1H), 4.91 (s, 2H), 6.24 (s, 2H), 7.13 (t, J=9 Hz, 2H), 7.33 (m, 1H), 7.50 (m, 2H), 7.95 (dd, J=9 Hz, 6 Hz, 2H), 8.46 (d, J=6 Hz, 1H); MS (DCI/NH₃) m/z 315 (M+H)⁺. Anal. Calcd C₁₈H₁₉FN₂O₂.1.4 C₄H₄O₄: C, 59.44; H, 5.20; N, 5.87. Found: C, 59.04; H, 5.10; N, 5.74.

### EXAMPLE 83D

### 2-{1-[2-(4-fluorophenyl)-2-(methoxyimino)ethyl]piperidin-3-ylpyridinium N-oxide

The product from Example 83C (38 mg, 0.12 mmol) and O-methylhydroxylamine hydrochloride (50.1 mg, 0.6 mmol) were combined in pyridine (5 mL) at room temperature and stirred overnight at ambient temperature for 12 hours. The mixture was concentrated under reduced pressure and partitioned between saturated NaHCO₃ and ethyl acetate. The organic extract was washed with brine, dried with Na₂SO₄, filtered, and the filtrate was concentrated under reduced pressure. The residue was purified by column chromatography (silica gel, 10:1 CH₂Cl₂/methanol) to afford the title compound. ¹H NMR (300 MHz, CDCl₃) δ 1.73 (m, 5H), 2.69 (m, 4H), 3.60 (m, 5H), 7.63 (m, 8H); MS (DCI/NH₃) m/z 344 (M+H)⁺.

### EXAMPLE 84

### 2-{1-[3-(4-fluorophenyl)-3-(methoxyimino)propyl]piperidin-3-yl}pyridinium N-oxide

### EXAMPLE 84A

### 2-{1-[3-(4-fluorophenyl)-3-oxopropyl]piperidin-3-yl}pyridinium N-oxide

The title compound was prepared using the procedure described in Example 83C except using 3-chloro-4′fluoropropiophenone instead of 2-chloro-4′-fluoroacetophenone. ¹H NMR (300 MHz, MeOH-d₄) δ 1.69 (m, 3H), 1.99 (m, 1H), 2.22 (m, 2H), 2.87 (m, 3H), 3.29 (m, 3H), 3.79 (m, 1H), 7.22 (m, 2H), 7.40 (m, 1H), 7.53 (m, 2H), 8.03 (m, 2H), 8.34 (d, J=5.5 Hz, 1H); MS (APCI) m/z 329 (M+H)⁺.

### EXAMPLE 84B

### 2-{1-[3-(4-fluorophenyl)-3-(methoxyimino)propyl]piperidin-3-yl}pyridinium N-oxide

The title compound was prepared using the procedure described in Example 83D except using the product from Example 84A instead of the product from Example 83C.

¹H NMR (300 MHz, CDCl₃) δ 1.66 (m, 3H), 1.95(m, 1H), 2.28 (m, 2H), 2.58 (m, 2H), 2.76 (m, 1H), 2.96 (m, 2H), 3.09 (m, 1H), 3.80 (m, 1H), 3.95 (s, 3H), 7.11 (m, 3H), 7.47 (d, J=9 Hz), 7.64 (m, 2H), 8.25 (dd, J=6 Hz, 3 Hz, 1H); MS (DCI/NH₃) m/z 358 (M+H)⁺. Anal Calcd for C₂₀H₂₄FN₃O₂.1.45 C₄H₄O₄: C, 58.94; H, 5.71; N, 7.99. Found: C, 58.99; H, 5.99; N, 7.86.

### EXAMPLE 85

### (1E)-1-(4-fluorophenyl)-2-[(2S)-2-methyl-4-pyridin-2-ylpiperazin-1-yl]ethanone O-methyloxime and

### (1Z)-1-(4-fluorophenyl)-2-[(2S)-2-methyl-4-pyridin-2-ylpiperazin-1-yl]ethanone O-methyloxime

### EXAMPLE 85A

### (3S)-3-methyl-1-pyridin-2-ylpiperazine

(S)-(+)-2-Methylpiperazine (0.50 g, 5.0 mmol) and 2-bromopyridine (5.0 mL, 50 mmol) were heated at 120° C. for 18 hours. The mixture was cooled to 22° C., diluted with water, and extracted with ethyl acetate. The organic phase was washed with dilute aqueous HCl (2×) and the combined aqueous layers were concentrated under reduced pressure. The residue was triturated with diethyl ether, dissolved in methanol, and azeotroped with dry toluene (2×) to provide the title compound which was used in the next step without further purification.

¹H NMR (300 MHz, DMSO-d₆) δ 1.30 (d, J=6.4 Hz, 3H), 3.17 (m, 2H), 3.41 (m, 3H), 4.36 (m, 2H), 6.93 (t, J=6.2 Hz, 1H), 7.28 (d, J=8.8 Hz, 1H), 7.90 (t, J=7.8 Hz, 1H), 8.13 (dd, J=5.6 Hz, 1.5 Hz, 1H), 9.17 (br s, 1H), 9.35 (br s, 1H); MS (DCI/NH₃) m/e 178 (M+H)⁺.

### EXAMPLE 85B

### 1-(4-fluorophenyl)-2-[(2S)-2-methyl-4-pyridin-2-ylpiperazin-1-yl]ethanone

The title compound was prepared using the procedure described in Example 3A except using the product from Example 85A instead of 1-(2-pyridinyl)piperazine.¹H NMR (300 MHz, CDCl₃) δ 1.20 (d, J=6.2 Hz, 3H,) 2.59 (m, 1H), 2.75 (m, 1H), 2.90 (dd, J=12.5 Hz, 9.4 Hz, 1H), 2.96 (dt, J=11.3 Hz, 3.4 Hz, 1H), 3.17 (m, 1H), 3.63 (d, J=16.2 Hz, 1H), 3.90 (m, 1H), 4.03 (m, 1H), 4.18 (d, J=16.2 Hz, 1H), 6.61 (dd, J=6.9 Hz, 5.0 Hz, 1H), 6.63 (d, J=8.7 Hz, 1H), 7.13 (t, J=8.6 Hz, 1H), 7.13 (m, 1H), 7.47 (m, 1H), 8.11 (dt, J=10.0 Hz, 5.0 Hz, 1H), 8.11 (dd, J=8.7 Hz, 5.6 Hz, 1H), 8.18 (dd, J=5.0 Hz, 1.3 Hz, 1H); MS (DCI/NH₃) m/z 314 (M+H)⁺.

### EXAMPLE 85C

### (1E)-1-(4-fluorophenyl)-2-[(2S)-2-methyl-4-pyridin-2-ylpiperazin-1-yl]ethanone O-methyloxime and

### (1Z)-1-(4-fluorophenyl)-2-[(2S)-2-methyl-4-pyridin-2-ylpiperazin-1-yl]ethanone O-methyloxime

The title compound was prepared using the procedure described in Example 1B except using the product from Example 85B instead of the product from Example 1A.

Z-isomer: ¹H NMR (300 MHz, CDCl₃) δ 1.21 (d, J=6.4 Hz, 3H), 2.35 (m, 1H), 2.54 (m, 1H), 2.78 (dt, J=11.9 Hz, 3.4 Hz, 1H), 2.80 (dd, J=12.6 Hz, 9.2 Hz, 1H), 2.97 (m, 1H), 3.68 (d, J=12.9 Hz, 1H), 3.73 (m, 1H) 3.80 (d, J=12.9 Hz, 1H), 3.92 (m, 1H), 3.97 (s, 3H), 6.58 (t, J=3.6 Hz, 1H), 6.59 (d, J=8 Hz, 1H), 7.03 (t, J=9 Hz, 1H), 7.03 (m, 1H), 7.44 (m, 1H), 7.84 (m, 1H), 7.84 (dd, J=9.0 Hz, 5.6 Hz, 1H), 8.16 (dd, J=5.8 Hz, 2.0 Hz, 1H); MS (DCI/NH₃) m/z 343 (M+H)⁺. Anal. calcd for C₁₉H₂₃FN₄O: C, 66.65; H, 6.77; N, 16.36. Found: C, 66.44; H, 6.67; N, 15.97

E-isomer: ¹H NMR (300 MHz, CDCl₃) δ 1.11 (d, J=6.4 Hz, 3H), 2.38 (m, 1H), 2.58 (m, 1 H), 2.79 (dd, J=12 Hz, 3.6 Hz, 1H), 2.93 (dt, J=12 Hz, 3.6 Hz, 1H), 3.07 (m, 1H), 3.17 (d, J=13 Hz, 1H), 3.80 (m, 1H), 3.82 (d, J=13 Hz, 1H), 3.88 (s, 3H), 3.89 (m, 1H), 6.58 (t, J=5.0 Hz, 1H), 6.61 (d, J=8 Hz, 1H), 7.06 (m, 2H), 7.44 (m, 1H), 7.61 (m, 2H), 8.17 (m, 1H); MS (DCI/NH₃) m/z 343 (M+H)⁺.

Maleate salt (white solid): ¹H NMR (300 MHz, DMSO-d₆) δ 1.17 (br s, 3H), 2.65 (m, 8H), 3.83 (s, 3H), 3.97 (m, 1H), 6.17 (s, 2H), 6.67 (dd, J=6.8 Hz, 5.1 Hz, 1H), 6.87 (d, 1H), 6.87 (d, J=8.5 Hz, 1H), 7.29 (t, J=9 Hz, 2H), 7.56 (m, 1H), 7.66 (dd, J=8.7 Hz, 5.6 Hz, 2H), 8.10 (dd, J=5 Hz, 1.4 Hz, 1H); Anal. calcd for C₁₉H₂₃FN₄O.1.1 C₄H₄O₄: C, 59.79; H, 5.87; N, 11.92. Found: C, 60.06; H, 6.00; N, 11.39.

### EXAMPLE 86

### (1E)-1-(4-chlorophenyl)-3-[(2S)-2-methyl-4-pyridin-2-ylpiperazin-1-yl]propan-1-one O-methyloxime and

### (1Z)-1-(4-chlorophenyl)-3-[(2S)-2-methyl-4-pyridin-2-ylpiperazin-1-yl]propan-1-one O-methyloxime

### EXAMPLE 86A

### 1-(4-chlorophenyl)-3-[(2S)-2-methyl-4-pyridin-2-ylpiperazin-1-yl]propan-1-one

The title compound was prepared using the procedure described in Example 2A except using the product from Example 85A instead of 1-(2-pyridinyl)piperazine. ¹H NMR (300 MHz, CDCl₃) δ 1.15 (d, J=6.2 Hz, 3H), 2.47 (td, J=1 Hz, 3 Hz, 1H), 2.57 (m, 1H), 2.79 (dd, J=12 Hz, 9.0 Hz, 1H), 2.83 (dd, J=8.7 Hz, 5.3 Hz, 1H), 2.96 (dt, J=11 Hz, 3.4 Hz, 1H), 3.13 (m, 3H), 3.25 (m, 1H), 3.95 (m, 2H), 6.60 (dd, J=7.2 Hz, 5.0 Hz, 1H), 6.63 (d, J=8.4 Hz, 1H), 7.44 (m, 2H) 7.47 (m, 1H), 7.90 (m, 2H) 8.18 (dd, J=5.0 Hz, 1.9 Hz, 1H); MS (DCI/NH₃) m/z 344.1 (M+H)⁺.

### EXAMPLE 86B

### (1E)-1-(4-chlorophenyl)-3-[(2S)-2-methyl-4-pyridin-2-ylpiperazin-1-yl]propan-1-one O-methyloxime and

### (1Z)-1-(4-chlorophenyl)-3-[(2S)-2-methyl-4-pyridin-2-ylpiperazin-1-yl]propan-1-one O-methyloxime

The title compounds were prepared using the procedure described in Example 1B except using the product from Example 86A instead of the product from Example 1A.

E-isomer: ¹H NMR (300 MHz, CDCl₃) δ 1.11 (d, J=6.4 Hz, 3H), 2.57 (m, 2H), 2.72 (m, 2H), 2.90 (m, 4H), 3.09 (m, 1H), 3.95 (m, 1H), 3.98 (s, 3H), 3.99 (m, 1H), 6.60 (m, 1H), 6.64 (d, J=8.8 Hz, 1H), 7.34 (m, 2H), 7.47 (m, 1H), 7.59 (m, 2H), 8.18 (m, 1H); MS (DCI/NH₃) m/z 373 (M+H)⁺.

Maleate salt: ¹H NMR (300 MHz, DMSO-d₆) δ 1.25 (d, J=5.4 Hz, 3H), 4.00 (m, 11H), 3.97 (s, 3H), 6.08 (s, 2H), 6.72 (dd, J=7.0 Hz, 4.9 Hz, 1H), 6.95 (d, J=8.5 Hz, 1H), 7.50 (m, 2H), 7.59 (m, 1H), 7.72 (m, 2H), 8.14 (dd, J=4.8 Hz, 1.4 Hz, 1H); Anal. calcd for C₂₀H₂₅ClN₄O.1.0 C₄H₄O₄: C, 58.95; H, 5.98; N, 11.46. Found: C, 58.77; H, 5.97; N, 11.15.

Z-isomer: ¹H NMR (300 MHz, DMSO-d₆) δ 0.85 (d, J=6.1 Hz, 3H), 2.22 (m, 1H), 2.33 (m, 2H), 2.68 (m, 4H), 2.83 (m, 1H), 2.96 (m, 1H), 3.72 (s, 3H), 3.85 (br d, J=12.2 Hz, 2H), 6.61 (d, J=7.1 Hz, 1H), 6.79 (d, J=8.5 Hz, 1H), 7.48 (m, 5H), 8.1 (m, 1H); MS (DCI/NH₃) m/z 373 (M+H)⁺.

Maleate salt: ¹H NMR (300 MHz, DMSO-d₆) δ 1.28 (d, J=5.1 Hz, 3H), 3.69 (m, 11H), 3.80 (s, 3H), 6.09 (s, 2H), 6.73 (dd, J=7 Hz, 5 Hz, 1H), 6.95 (d, J=8.8 Hz, 1H), 7.58 (m, 5H), 8.15 (dd, J=4.6 Hz, 1.5 Hz, 1H); Anal. calcd for C₂₀H₂₅ClN₄O.1.2 C₄H₄O₄: C, 58.16; H, 5.86; N, 10.94. Found: C, 58.43; H, 6.04; N, 10.77.

### EXAMPLE 87

### (1E)-1-(4-chlorophenyl)-3-(3-methyl-3′,6′-dihydro-2,4′-bipyridin-1′(2′H)-yl)propan-1-one O-methyloxime and

### (1Z)-1-(4-chlorophenyl)-3-(3-methyl-3′,6′-dihydro-2,4′-bipyridin-1′(2′H)-yl)propan-1-one O-methyloxime

### EXAMPLE 87A

### tert-butyl 3-methyl-3′,6′-dihydro-2,4′-bipyridine-1′(2′H)-carboxylate

The title compound was prepared using the procedure described in Example 88B except using 3-methyl-2-pyridinyl zinc bromide instead of 2-pyridinyl zinc bromide.

¹H NMR (300 MHz, DMSO-d₆) δ 1.43 (s, 9H), 2.32 (s, 1H), 2.46 (m, 2H), 3.54 (t, 2H), 4.00 (m, 2H), 5.84 (m, 1H), 7.28 (dd, J=9 Hz, 3 Hz, 1H), 7.62 (d, J=9 Hz, 1H), 8.36 (m, 2H).

### EXAMPLE 87B

### 3-methyl-1′,2′,3′,6′-tetrahydro-2,4′-bipyridine

The product from Example 87A (4.5 g, 16 mmol) in ethyl acetate (100 mL) was treated with HCl gas bubbled through the mixture at −78° C. for 15 minutes. The mixture was allowed to warm to room temperature and was filtered. The filter cake was washed with ethyl acetate and dried under high vaccum to provide the dihydrochloride of the title compound. MS (DCI—NH₃) m/z 175 (M+H)⁺.

### EXAMPLE 87C

### 1-(4-chlorophenyl)-3-(3-methyl-3′,6′-dihydro-2,4′-bipyridin-1′(2′H)-yl)propan-1-one

The title compound was prepared using the procedure described in Example 2A except using the product from Example 87B instead of 1-(2-pyridinyl)piperazine. ¹H NMR (300 MHz, CDCl₃) δ 2.37 (s, 3H), 2.61 (m, 2H), 2.81 (d, J=5.4 Hz, 1H), 2.82 (t, J=5.6 Hz, 1H), 2.99 (t, J=7.3 Hz, 1H), 3.00 (d, J=7.8 Hz, 1H), 3.26 (m, 4H), 5.78 (m, 1H), 7.06 (dd, J=7.5 Hz, 4.8 Hz, 1H), 7.45 (m, 2H), 7.47 (m, 1H), 7.92 (m, 2H), 8.41 (dd, J=4.8 Hz, 1.7 Hz, 1H); MS (DCI/NH₃) m/z 341.1 (M+H)⁺.

### EXAMPLE 87D

### (1E)-1-(4-chlorophenyl)-3-(3-methyl-3′,6′-dihydro-2,4′-bipyridin-1′(2′H)-yl)propan-1-one O-methyloxime and

### (1Z)-1-(4-chlorophenyl)-3-(3-methyl-3′,6′-dihydro-2,4′-bipyridin-1′(2′H)-yl)propan-1-one O-methyloxime

The title compounds were prepared using the procedure described in Example 1B except using the product from Example 87C instead of the product from Example 1A.

E-isomer: ¹H NMR (300 MHz, CDCl₃) δ 2.36 (s, 3H), 2.58 (m, 2H), 2.69 (m, 2H), 2.78 (d, J=5.4 Hz, 1H), 2.79 (t, J=5.6 Hz, 1H), 3.01 (m, 2H), 3.24 (q, J=2.8 Hz, 2H), 3.99 (s, 3H), 5.78 (m, 1H), 7.06 (dd, J=7.8 Hz, 4.8 Hz, 1H), 7.34 (m, 2H), 7.47 (m, 1H), 7.62 (m, 2H), 8.41 (dd, J=4.8 Hz, 1.4 Hz, 1H); MS (DCI/NH₃) m/z 370 (M+H)⁺. Anal. calcd for C₂₁H₂₄ClN₃O.0.3 H₂O: C, 67.21; H, 6.61; N, 11.20. Found: C, 67.30; H, 6.42; N, 11.20.

Z isomer: ¹H NMR (300 MHz, CDCl₃) δ 2.35 (s, 3H), 2.56 (m, 2H), 2.62 (m, 2H), 2.72 (t, J=5.6 Hz, 2H), 2.79 (m, 2H), 3.16 (q, J=2.8 Hz, 2H), 3.83 (s, 3H), 5.75 (m, 1H), 7.06 (dd, J=7.5 Hz, 4.8 Hz, 1H), 7.36 (m, 4H), 7.48 (m, 1H), 8.41 (dd, J=4.4 Hz, 1.4 Hz, 1H); MS (DCI/NH₃) m/z 370.2 (M+H)⁺. Anal. calcd for C₂₁H₂₄ClN₃O: C, 68.19; H, 6.54; N, 11.36. Found: C, 67.99; H, 6.27; N, 11.53.

### EXAMPLE 88

### (1E)-1-(4-chlorophenyl)-3-(4-pyridin-2-ylpiperidin-1-yl)propan-1-one O-methyloxime and

### (1Z)-1-(4-chlorophenyl)-3-(4-pyridin-2-ylpiperidin-1-yl)propan-1-one O-methyloxime

### EXAMPLE 88A

### tert-butyl 4-{[(trifluoromethyl)sulfonyl]oxy}-3,6-dihydropyridine-1(2H)-carboxylate

Diisopropylamine (13.4 mL. 96 mmol) in THF (350 mL) at −78° C. was treated with 1.6 M nBuLi in hexane (60 mL, 96 mmol). After stirring for 5 minutes, the mixture was treated with tert-butoxycarbonyl-4-piperidone (16 g, 80 mmol) in THF (100 mL). After 10 minutes. the mixture was treated with a solution of N-phenyltrifluoromethanesulfonimide (31.4 g, 88 mmol). After 30 minutes of stirring at −78° C., the mixuture was allowed to warn to room temperature (˜1.5 hours). The mixture was quenched with saturated NaHCO₃ and extracted with diethyl ether. The organic layer was, washed with 5% citric acid, washed with 1N NaOH (4×200 mL), washed with water (2×200 mL), washed with brine (200 mL), dried over MgSO₄, filtered, and the filtrate was concentrated under reduced pressure. The residue was purified by flash chromatography (hexanes: ethyl acetate 8:2) to provide the title compound. ¹H NMR (300 MHz, DMSO-d₆) δ 1.41 (s, 9H), 2.41 (m, 2H), 3.54 (t, 2H), 3.98 (m, 2H), 6.02 (m, 1H).

### EXAMPLE 88B

### tert-butyl 3′,6′-dihydro-2,4′-bipyridine-1′(2′H)-carboxylate

The product from Example 88A (18 g, 54 mmol) in THF (˜200 mL) was treated with 2-pyridinylzinc bromide 0.5 M solution in THF (from Aldrich Co.) (124 mL, 62.5 mmol) and Pd(PPh₃)₄ (from Strem Chemicals) (625 mg) and at 60° C. for 90 minutes. The mixture was allowed to cool to room temperature, concentrated under reduced pressure, and treated with ethyl acetate (300 mL) and 1N NaOH (200 mL). The mixture was filtered and the organic layer was separated, washed with brine (300 mL), dried over anhydrous MgSO₄, filtered, and the filtrate was concentrated under reduced pressure. The residue was purified by flash chromatography (hexanes:ethyl acetate 6:4) to afford the title compound.

¹H NMR (300 MHz, DMSO-d₆) δ 1.43 (s, 9H), 2.56 (m, 2H), 3.54 (t, 2H), 4.04 (m, 2H), 6.08 (m, 1H), 7.25 (dd, J=9 Hz,1H), 7.56 (d, J=9 Hz, 1H), 7.77 (m, 1H), 8.54 (m, 1H); MS (DCI—NH₃) m/z 259 (M+H)⁺.

### EXAMPLE 88C

### tert-butyl 4-pyridin-2-ylpiperidine-1-carboxylate

The product from Example 88B (9.0 g, 35 mmol) was treated with 10% Pd/C (900 mg) under a hydrogen atmosphere (60 psi pressure) at room temperature for 1.5 hours to provide the title compound. ¹H NMR (300 MHz, DMSO-d₆) δ 1.41 (s, 9H), 1.58 (m, 2H), 1.81 (m, 2H), 2.85 (m, 3H), 4.06 (m, 2H), 7.20 (dd, J=9 Hz, 3 Hz, 1H), 7.28 (d, J=9 Hz, 1H), 7.70 (m, 1H), 8.48 (m, 1H).

### EXAMPLE 88D

### 2-piperidin-4-ylpyridine

The product from Example 88C (2.8 g, ˜11 mmol)) in 1,4-dioxane (15 mL) was treated with 4N HCl in 1,4-dioxane (25 mL) at room temperature with stirring for 1 hour. The mixture was concentrated under reduced pressure and triturated with diethyl ether. The mixture was filtered and the filter cake was washed with ethyl acetate and dried under reduced pressure to provide the title compound as the hydrochloride salt. MS (DCI—NH₃) m/z 163 (M+H)⁺.

### EXAMPLE 88E

### 1-(4-chlorophenyl)-3-(4-pyridin-2-ylpiperidin-1-yl)propan-1-one

The title compound was prepared using the procedure described in Example 2A except using the product from Example 88D instead of 1-(2-pyridinyl)piperazine. MS (DCI—NH₃) m/z 329 (M+H)⁺.

### EXAMPLE 88F

### (1E)-1-(4-chlorophenyl)-3-(4-pyridin-2-ylpiperidin-1-yl)propan-1-one O-methyloxime and

### (1Z)-1-(4-chlorophenyl)-3-(4-pyridin-2-ylpiperidin-1-yl)propan-1-one O-methyloxime

The title compounds were prepared using the procedure described in Example 1B except using the product from Example 88E instead of the product from Example 1A.

E-isomer: ¹H NMR (300 MHz, DMSO-d₆) δ 1.71 (m, 4H), 2.05 (t, J=10.5 Hz, 2H), 2.45 (t, J=7.5 Hz, 2H), 2.61 (m, 1H), 2.92 (m, 4H), 3.92 (s, 3H,), 7.19 (dd, J=7.5 Hz, 6 Hz, 1H), 7.26 (d, J=9 Hz, 1H), 7.47 (m, 2H), 7.69 (m, 3H), 8.48 (d, 1H); MS (DCI—NH₃) m/z 358 (M+H)⁺. Anal. calcd for the maleate salt, C₂₄H₂₈ClN₃O₅: C, 60.82; H, 5.95, N, 8.87. Found: C, 61.84, H, 5.98, N, 9.11

Z-isomer: ¹H NMR (300 MHz, DMSO-d₆) δ 1.71 (m, 4H), 1.98 (t, J=10.5 Hz, 2H), 2.36 (t, J=7.5 Hz, 2H), 2.60 (m, 1H), 2.68 (t, J=7.5 Hz, 2H), 2.88 (d, J=10.5 Hz, 2H), 3.71 (s, 3H,), 7.19 (dd, J=7.5, 6 Hz, 1H), 7.25 (d, J=9 Hz, 1H), 7.45 (m, 4H), 7.69 (m, 1H), 8.48 (d, 1H); MS (DCI—NH₃) m/z 358 (M+H)⁺. Anal. calcd for C₂₀H₂₄ClN₃O.0.1H₂O: C, 66.79; H, 6.78, N, 11.68. Found: C, 66.43, H, 6.78, N, 11.59.

### EXAMPLE 89

### 1-(4-fluorophenyl)-2-(4-pyridin-2-ylpiperidin-1-yl)ethanone methylhydrazone

The product of Example 3A (160 mg, 0.53 mmol), in 1,4-dioxane (10 mL), was treated with methylhydrazine (0.028 mL, 0.53 mmol) and acetic acid (0.04 mL) at room temperature and allowed to stir for 48 hours. The mixture was concentrated under reduced pressure and the residue was partitioned between water and ethyl acetate (30 mL). The organic layer was washed with brine (2×30 mL), dried over MgSO₄, filtered, and the filtrate was concentrated under reduced pressure. The residue was purified by flash chromatography (ethyl acetate:hexanes, 1:1) to provide the title compound. ¹H NMR (300 MHz, DMSO-d₆) δ 1.75 (m, 4H), 2.16 (m, 2H), 2.65 (m, 1H), 2.90 (m, 5H), 3.52 (s, 2H), 7.15 (m, 3H), 7.27 (d, J=7.5 Hz, 1H), 7.68 (m, 3H), 7.80 (m, 1H), 8.49 (m, 1H); MS (DCI—NH₃) m/z 327 (M+H)⁺.

### EXAMPLE 90

### (1E)-1-pyridin-3-yl-3-(4-pyridin-2-ylpiperazin-1-yl)propan-1-one O-methyloxime and

### (1Z)-1-pyridin-3-yl-3-(4-pyridin-2-ylpiperazin-1-yl)propan-1-one O-methyloxime

### EXAMPLE 90A

### 1-pyridin-3-yl-3-(4-pyridin-2-ylpiperazin-1-yl)propan-1-one

3-Acetylpyridine (1.21 g, 10 mmol), 1-(2-pyridinyl)piperazine (1.1 mL, 7 mmol), paraformaldehyde (300 mg, 10 mmol) and concentrated HCl (2 mL, 23 mmol) were combined in isopropanol (20 mL) and refluxed for 26 hours. The mixture was concentrated under reduced pressure, treated with saturated NaHCO₃, and extracted with ethyl acetate. The ethyl acetate extract was washed with brine, dried with anhydrous MgSO₄, filtered, and the filtrate was concentrated under reduced pressure. The residue was purified by column chromatography (silica gel, ethyl acetate) to provide the title compound. MS (DCI/NH₃) m/z 297 (M+H)⁺.

### EXAMPLE 90B

### 1(1E)-1-pyridin-3-yl-3-(4-pyridin-2-ylpiperazin-1-yl)propan-1-one O-methyloxime and

### (1Z)-1-pyridin-3-yl-3-(4-pyridin-2-ylpiperazin-1-yl)propan-1-one O-methyloxime

The product from Example 90A (1.3 g, 4.4 mmol) and O-methylhydroxylamine hydrochloride (830 mg, 10 mmol) were combined in pyridine (30 mL) at room temperature and stirred at ambient temperature for 12 hours. The mixture was concentrated under reduced pressure, treated with saturated NaHCO₃, and extracted with ethyl acetate. The ethyl acetate layer was washed with brine, dried with MgSO₄, filtered, and the filtrate was concentrated under reduced pressure. The residue was purified by column chromatography (silica gel, CH₂Cl₂-acetone 4:1) to provide the title compounds.

Maleate salt (2.5:1 ratio), ¹H NMR (300 MHz, DMSO-d₆) δ 3.23 (m, 12H), 4.82 and 4.98 (2s, 1:3, 3H), 6.17 (s, 5H), 6.75 (m, 1H), 6.95 (d, J=7 Hz, 1H), 7.44 (m, 1H), 7.62 (m, 1H), 7.94 and 8.07 (2m, 1:3, 1H), 8.17 (m, 1H), 8.61 and 8.65 (2m, 1:2, 1H), 8.72 and 8.90 (2m, 1:3, 1H); MS (DCI/NH₃) m/z 326 (M+H)⁺. Anal. calcd for C₁₈H₂₃ClN₅O.2.5C₄H₄O₄: C, 54.63; H, 5.40; N, 11.38. Found: C, 54.53; H, 5.35; N, 11.18.

### EXAMPLE 91

### 1-(4-chlorophenyl)-3-(4-pyridin-2-ylpiperazin-1-ylpropan-1-one O-(2-hydroxyethyl)oxime

The product from Example 22 (344 mg, 1 mmol) and KOH (74 mg, 1.5 mmol) were combined in DMSO (15 mL) and H₂O (5 mL), treated with 2-bromoethanol (150 mg, 1.2 mmol), and stirred at room temperature for 4 hours. The mixture was poured into water (25 mL) and extracted with ethyl acetate. The ethyl acetate layer was washed with water, brine, dried over anhydrous MgSO₄, filtered, and the filtrate was concentrated under reduced pressure. The residue was purified by column chromatography to provide the title compound. ¹H NMR (300 MHz, DMSO-d₆) δ 2.40 (m, 2H), 2.48 (m, 4H), 3.72 and 3.95 (2t, 1:5, J=6 Hz, 2H), 3.41 (t, J=6 Hz, 4H), 3.55 and 3.65 (2q, 1:5, J=6 Hz, 2H), 3.97 and 4.15 (2t, 1:5, J=6 Hz, 2H), 4.58 and 4.67 (2t, J=6 Hz, 1H), 6.62 (m, 1H), 6.80 (d, J=7 Hz, 1H), 7.50 (m, 3H), 7.67 (m, 2H), 8.10 (m, 1H); MS (DCI/NH₃) m/z 389 (M+H)⁺. Anal. calcd for C₂₀H₂₅ClN₄O₂: C, 61.77; H, 6.48; N, 14.41. Found: C, 61.50; H, 6.27; N, 14.12.

### EXAMPLE 92

### (1E)-1-(4-chlorophenyl)-3-[4-(5-hydroxypyridin-2-yl)piperazin-1-yl]propan-1-one O-methyloxime and

### (1Z)-1-(4-chlorophenyl)-3-[4-(5-hydroxypyridin-2-yl)piperazin-1-yl]propan-1-one O-methyloxime

### EXAMPLE 92A

### 1-[5-(benzaloxy)pyridin-2-yl]piperazine

tert-Butyl 4-[5-(benzyloxy)pyridin-2-yl]piperazine-1-carboxylate (1.5 g, 4 mmol) in CH₂Cl₂ (10 mL) was treated with TFA (3 mL) at 0° C. and stirred for 1 hour. The mixture was allowed to warm to room temperature and concentrated under reduced pressure to provide the title compound which was used in the next step without further purification. MS (DCI/NH₃) m/z 270 (M+H)⁺.

### EXAMPLE 92B

### 3-{4-[5-(benzyloxy)pyridin-2-yl]piperazin-1-yl}-1-(4-chlorophenyl)propan-1-one

The product from Example 92A (1.8 g, ˜2.3 mmol), K₂CO₃ (300 mg, 2.3 mmol), 3,4′-dichloropropiophenone (460 mg, 2.3 mmol) and n-Bu4N⁺HSO₄⁻(20 mg) were combined in toluene (20 mL) and refluxed for 10 hours at 75° C. The mixture was allowed to cool to room temperature, diluted with ethyl acetate (30 mL), and the organics separated. The organics were washed with water, brine, dried over anhydrous MgSO₄, filtered, and the filtrate was concentrated under reduced pressure to provide the title compound. ¹H NMR (300 MHz, DMSO-d₆) δ 2.70 (t, J=7 Hz, 2H), 3.22 (t, J=7 Hz, 2H), 3.30 (m, 8H), 5.05 (s, 2H), 6.90 (d, J=9 Hz, 1H), 7.38 (m, 6H), 7.60 (m, 2H), 7.92 (d, J=3 Hz, 1H), 8.02 (m, 2H); MS (DCI/NH₃) m/z 436 (M+H)⁺.

### EXAMPLE 92C

### 3-{4-[5-(benzyloxy)pyridin-2-yl]piperazin-1-yl}-1-(4-chlorophenyl)propan-1-one O-methyloxime

The product from Example 92B (435 mg, 1 mmol) and methoxylamine hydrochloride (420 mg, 5 mol) were combined in pyridine (15 mL) and stirred at room temperature for 18 hours. The mixture was concentrated under reduced pressure and the residue treated with ethyl acetate and water. The organic layer was separated, washed with water, brine, dried over anhydrous MgSO₄, filtered, and the filtrate was concentrated under reduced pressure. The residue was purified by column chromatography (CH₂Cl₂:acetone 4:1) to provide the title compound. MS (DCI/NH₃) m/z 465 (M+H)⁺.

### EXAMPLE 92D

### 1-(4-chlorophenyl)-3-[4-(5-hydroxypyridin-2-yl)piperazin-1-yl]propan-1-one

The product from Example 92C (420 mg, 0.9 mmol) was treated with 10% Pd/C (83 mg) in methanol (55 mL) under a hydrogen atmosphere (60 psi) at room temperature for 76 minutes. The mixture was filtered, the filter cake washed with methanol, and the filtrate was concentrated under reduced pressure. The residue was purified by column chromatography (CH₂Cl₂:EtOH 7:1) to provide the title compound. MS (DCI/NH₃) m/z 346 (M+H)⁺.

### EXAMPLE 92E

### (1E)-1-(4-chlorophenyl)-3-[4-(5-hydroxypyridin-2-yl)piperazin-1-yl]propan-1-one O-methyloxime and

### (1Z)-1-(4-chlorophenyl)-3-[4-(5-hydroxypyridin-2-yl)piperazin-1-yl]propan-1-one O-methyloxime

The product from Example 92D (190 mg, 0.55 mmol) and methoxylamine hydrochloride (250 mg, 3 mol) were combined in pyridine (15 mL) and stirred at room temperature for 16 hours. The mixture was concentrated under reduced pressure, treated with saturated NaHCO₃, and extracted with ethyl acetate. The organic layer was separated, washed with water, brine, dried over anhydrous MgSO₄, filtered, and the filtrate was concentrated under reduced pressure. The residue was purified by column chromatography (ethyl acetate) to provide the title compound. MS (DCI/NH₃) m/z (M+H)⁺.

E-isomer, maleate salt: ¹H NMR (300 MHz, DMSO-d₆) δ 3.25 (m, 12H), 3.96 (s, 3H), 6.05 (s, 2H), 6.83 (d, J=7 Hz, 1H), 7.11 (d-d, J=3 Hz, 9 Hz, 1H), 7.32 (d, J=9 Hz, 2H), 8.72 (d, J=9 Hz, 2H), 7.77 (d, J=3 Hz, 1H), 9.14 (s, 1H); MS (DCI/NH₃) m/z 375 (M+H)⁺.

Z-isomer, maleate salt: ¹H NMR (300 MHz, DMSO-d₆) δ 3.00 (m, 2H), 3.30 (m, 10H), 3.80 (s, 3H), 6.08 (s, 2H), 6.83 (d, J=7 Hz, 11H), 7.11 (d-d, J=3 Hz, 9 Hz, 1H), 7.54 (s, 4H), 7.77 (d, J=3 Hz, 1H), 9.15 (s, 1H); MS (DCI/NH₃) m/z 375 (M+H)⁺.

### EXAMPLE 93

### 1-(4-fluorophenyl)-2-[4-(5-hydroxypyridin-2-yl)piperazin-1-yl]ethanone O-methyloxime

### EXAMPLE 93A

### benzyl 4-(5-methoxypyridin-2-yl)piperazine-1-carboxylate

Benzyl 4-(5-hydroxypyridin-2-yl)piperazine-1-carboxylate (3.2 g, 19 mmol), anhydrous K₂CO₃ (2.8 g, 20 mmol), and iodomethane (1 mL) were combined in acetone (50 mL) and refluxed at 50° C. for 12 hours. The mixture was concentrated under reduced pressure and the residue was partitioned between ethyl acetate and water. The acetate layer was washed with brine, dried over anhydrous MgSO₄, filtered, and the filtrate was concentrated under reduced pressure. The crude product was purified by column chromatography to provide the title compound. MS (DCI/NH₃) m/z 328 (M+H)⁺.

### EXAMPLE 93B

### 6-piperazin-1-ylpyridin-3-ol

The product from Example 93A (2.65 g, 8.1 mmol) in acetic acid (10 mL) was treated with a saturated solution of HBr in acetic acid (10 mL) at room temperature for 30 minutes. The mixture was concentrated under reduced pressure and the residue was triturated with diethyl ether (3×20 mL) to provide the title compound which was used in next step without further purification.

### EXAMPLE 93C

### 1-(4-fluorophenyl)-2-[4-(5-methoxypyridin-2-yl)piperazin-1-yl]ethanone

The product from Example 93B (360 mg, ˜1 mmol), K₂CO₃ (560 mg, 4 mmol), 4-fluorophenacyl chloride 175 mg, 1 mmol) and n-Bu₄N⁺HSO₄⁻ (20 mg) were combined in toluene (20 mL) and refluxed for 5 hours at 60-70° C. The mixture was allowed to cool to room temperature and diluted with ethyl acetate (30 mL). The organics were separated, washed with water, brine, dried over anhydrous MgSO₄, filtered, and the filtrate was concentrated under reduced pressure. The residue was chromatographed (hexanes:ethyl acetate 1:1) to provide the title compound. MS (DCI/NH₃) m/z 330 (M+H)⁺.

### EXAMPLE 93D

### 1-(4-fluorophenyl)-2-[4-(5-hydroxypyridin-2-yl)piperazin-1-yl]ethanone O-methyloxime

The product from Example 93C (93 mg, 0.28 mmol) in methylene chloride (10 mL) at −78° C. was treated with BBr₃ (1M solution in CH₂Cl₂) (0.9 mL, 0.9 mmol). The cooling bath was removed and the mixture was stirred for 1 hour, treated with saturated solution of NaHCO₃, and extracted with ethyl acetate. The ethyl acetate extract was washed with brine, dried over MgSO₄, filtered, and the filtrate was concentrated under reduced pressure. The residue was dissolved in pyridine (20 mL) and treated with methoxylamine hydrochloride (83 mg, 1 mmol). The mixture was stirred for 16 hours at room temperature, concentrated under reduced pressure, treated with saturated NaHCO₃, and extracted with ethyl acetate. The ethyl acetate layer was washed with brine and concentrated under reduced pressure. The residue was purified by column chromatography (CH₂Cl₂:acetone 4:1) to provide the title compound.

¹H NMR (300 MHz, DMSO-d₆) δ 2.57 (m, 4H), 3.20 (m, 2H), 3.65 (s, 2H), 3.92 (s, 3H), 6.65 (d, J=7 Hz, 1H), 7.03 (d-d, J=3 Hz, 9 Hz, 1H), 7.20 (t, J=9 Hz, 2H), 7.70 (d, J=3 Hz, 1H), 7.92 (d-d, J=6 Hz, 9 Hz, 2H), 8.94 (s, 1H); MS (DCI/NH₃) m/z 345 (M+H)⁺.

### EXAMPLE 94

### (1E)-1-(4-chlorophenyl)-3-[4-(5-hydroxypyridin-2-yl)piperazin-1-yl]propan-1-one O-methyloxime and

### (1Z)-1-(4-chlorophenyl)-3-[4-(5-hydroxypyridin-2-yl)piperazin-1-yl]propan-1-one O-methyloxime

### EXAMPLE 94A

### 1-(4-chlorophenyl)-3-[4-(5-methoxypyridin-2-yl)piperazin-1-yl]propan-1-one

The product from Example 93B (1.1 g, 3 mmol), K₂CO₃ (420 mg, 3 mmol), 3,4′-dichloropropiophenone (600 mg, 3 mmol) and n-Bu₄N⁺HSO₄⁻ (25 mg) were combined in toluene (40 mL) and refluxed for 10 hours at 75° C. The mixture was allowd to cool to room temperature and diluted with ethyl acetate (30 mL). The organics were separated, washed with water, brine, dried over anhydrous MgSO₄, filtered, and the filtrate was concentrated under reduced pressure. The residue was purified by column chromatography to provide the title compound. MS (DCI/NH₃) m/z 360 (M+H)⁺.

### EXAMPLE 94B

### 1-(4-chlorophenyl)-3-[4-(5-hydroxypyridin-2-yl)piperazin-1-yl]propan-1-one

The product from Example 94A (570 mg, 1.85 mmol) in CH₂Cl₂ (30 mL) at −20° C. was treated with 1N BBr₃ in methylene chloride (10 mL, 10 mmol). The mixture was allowed to warm to ambient temperature and stir for 3 hours. The mixture was washed with NaHCO₃, brine, dried over anhydrous MgSO₄, filtered, and the filtrate was concentrated under reduced pressure to provide the title compound. MS (DCI/NH₃) m/z 346 (M+H)⁺.

### EXAMPLE 94C

### (1E)-1-(4-chlorophenyl)-3-[4-(5-hydroxypyridin-2-yl)piperazin-1-yl]propan-1-one O-methyloxime and

### (1Z)-1-(4-chlorophenyl)-3-[4-(5-hydroxypyridin-2-yl)piperazin-1-ylpropan-1-one O-methyloxime

The title compounds were prepared using the procedure described in Example 92E except using was followed to provide the title compounds. MS (DCI/NH₃) m/z 375 (M+H)⁺ for both isomers.

### EXAMPLE 95

### (1E)-1-(4-chlorophenyl)-2-hydroxy-3-(4-pyridin-2-ylpiperazin-1-yl)propan-1-one O-methyloxime and

### (1Z)-1-(4-chlorophenyl)-2-hydroxy-3-(4-pyridin-2-ylpiperazin-1-yl)propan-1-one O-methyloxime

### EXAMPLE 95A

### 1-(4-chlorophenyl)-1,1-dimethoxy-3-(4-pyridin-2-ylpiperazin-1-yl)propan-2-ol

The product from Example 2A (522 mg, 1.6 mmol) and iodobenzene diacetate (PhI(OAc)₂ (547 mg, 1.7 mmol) were combined in methanol (25 mL) and treated with a solution of KOH (297 mg, 5.3 mmol) in methanol (5 mL) dropwise. The mixture was stirred for 5 hours at room temperature, concentrated under reduced pressure and the residue partitioned between ethyl acetate and water. The organic layer was separated, washed with brine, dried over anhydrous MgSO₄, fitlered, and the filtrate was concentrated under reduced pressure to provide the title compound. An analytical sample was purified by column chromatography (ethyl acetate). ¹H NMR (300 MHz, DMSO-d₆) δ 2.42 (m, 4H), 3.13 (s, 3H), 3.20 (s, 3H), 3.41 (m, 6H), 4.05 (m, 1H), 4.78 (d, J=6 Hz, 1H), 6.60 (d-d, J=4.5 Hz, 7 Hz, 1H), 6.77 (d, J=9 Hz, 1H), 7.40 (s, 4H), 7.50 (m, 1H), 8.08 (m, 1H); MS (DCI/NH₃) m/z 392 (M+H)⁺. Anal. calcd for C₂₀H₂₆ClN₃O₃: C, 61.30; H, 6.69; N, 10.72. Found: C, 61.41; H, 6.83; N, 10.92.

### EXAMPLE 95B

### 1-(4-chlorophenyl)-2-hydroxy-3-(4-pyridin-2-ylpiperazin-1-yl)propan-1-one

The crude product from Example 95A (570 mg, ˜1.5 mmol) in chloroform (20 mL) was treated at room temperature with 5% H₂SO₄ (15 mL) and stirred for 18 hours. The mixture was treated with saturated NaHCO₃ to pH 9. The organic layer was separated, washed with brine, dried over anhydrous MgSO₄, filtered, and the filtrate was concentrated under reduced pressure to afford the title compound. MS (DCI/NH₃) m/z 346 (M+H)⁺.

### EXAMPLE 95C

### (1E)-1-(4-chlorophenyl)-2-hydroxy-3-(4-pyridin-2-ylpiperazin-1-yl)propan-1-one O-methyloxime and

### (1Z)-1-(4-chlorophenyl)-2-hydroxy-3-(4-pyridin-2-ylpiperazin-1-yl)propan-1-one O-methyloxime

Methoxylamine hydrochloride (410 mg, 5 mmol) and the product from Example 95B (344 mg, ˜1 mmol) were combined in pyridine (10 mL) and stirred at room temperature for 14 hours. The mixture was concentrated under reduced pressure and partitioned between saturated solution of NaHCO₃ and ethyl acetate. The ethyl acetate layer was separated, washed with brine, dried over anhydrous MgSO₄, filtered, and the filtrate was concentrated under reduced pressure. The residue was purified by column chromatography (CH₂Cl₂:acetone 4:1) to provide the title compounds.

E-isomer, maleate salt: ¹H NMR (300 MHz, DMSO-d₆) δ 3.30 (m, 10H), 3.78 (s, 3H), 4.85 (m, 1H), 6.06 (s, 2H), 6.72 (d-d, J=4.5 Hz, 7 Hz, 1H), 6.91 (d, J=7 Hz, 1H), 7.44 (d, J=9 Hz, 2H), 7.52 (d, J=9 Hz, 1H), 7.60 (m, 1H), 8.15 (m, 1H); MS (DCI/NH₃) m/z 375 (M+H)⁺. Anal. calcd for C₁₉H₂₃ClN₄O₂.C₄H₄O₄: C, 56.27; H, 5.54; N, 11.41. Found: C, 56.60; H, 5.81; N, 11.08.

Z-isomer, maleate salt: ¹H NMR (300 MHz, DMSO-d₆) δ 3.30 (m, 10H), 4.95 (s, 3H), 4.35 (m, 1H), 5.56 (broad d, J=7 Hz, 1H), 6.11 (s, 3H), 6.74 (d-d, J=4.5 Hz, 7 Hz, 1H), 6.93 (d, J=7 Hz, 1H), 7.48 (d, J=9 Hz, 2H), 7.60 (m, 1H), 7.70 (d, J=9 Hz, 1H), 8.15 (m, 1H); MS (DCI/NH₃) m/z 375 (M+H)⁺. Anal. calcd for C₁₉H₂₃ClN₄O₂.1.5C₄H₄O₄: C, 54.70; H, 5.32; N, 10.21. Found: C, 54.47; H, 5.72; N, 9.81.

### EXAMPLE 96

### (1E)-1-(4-chlorophenyl)-3-(4-pyrimidin-2-ylpiperazin-1-yl)propan-1-one O-methyloxime and

### (1Z)-1-(4-chlorophenyl)-3-(4-pyrimidin-2-ylpiperazin-1-yl)propan-1-one O-methyloxime

### EXAMPLE 96A

### 1-(4-chlorophenyl)-3-(4-pyrimidin-2-ylpiperazin-1-yl)propan-1-one

The title compound was prepared using the procedure described in Example 2A except using 1-(2-pyrimidinyl)piperazine instead of 1-(2-pyridinyl)piperazine to provide the title compound. MS (DCI/NH₃) m/z 331 (M+H)⁺.

### EXAMPLE 96B

### (1E)-1-(4-chlorophenyl)-3-(4-pyrimidin-2-ylpiperazin-1-yl)propan-1-one O-methyloxime and

### (1Z)-1-(4-chlorophenyl)-3-(4-pyrimidin-2-ylpiperazin-1-yl)propan-1-one O-methyloxime

The title compounds were prepared using the procedure described in Example 90B except using the product from Example 96A instead of the product from EXAMPLE 90A.

E-isomer, maleate salt: ¹H NMR (300 MHz, DMSO-d₆) δ 2.43 (m, 4H), 2.50 (m, 2H), 2.97 (m, 2H), 3.67 (t, J=6 Hz, 4H), 3.92 (s, 3H), 6.60 (t, J=6 Hz, 1H), 7.47 (d, J=9 Hz, 2H), 7.66 (d, J=9 Hz, 2H), 8.13 (d, J=4.5 Hz, 2H); MS (DCI/NH₃) m/z 360 (M+H)⁺. Anal. calcd for C₁₈H₂₂ClN₅O: C, 60.08; H, 6.16; N, 19.46. Found: C, 60.01; H, 6.03; N, 19.21.

Z-isomer, maleate salt: ¹H NMR (300 MHz, DMSO-d₆) δ 3.20 (m, 12H), 3.79 (s, 3H), 6.07 (s, 2H), 6.74 (t, J=6 Hz, 1H), 7.52 (s, 4H), 8.42 (d, J=4.5 Hz,2H); MS (DCI/NH₃) m/z 360 (M+H)⁺. Anal. calcd for C₁₈H₂₂ClN₅O.C₄H₄O₄: C, 55.52; H, 5.51; N, 14.72. Found: C, 55.18; H, 5.09; N, 14.00.

### EXAMPLE 97

### 1-(3-methylphenyl)-3-(4-pyridin-2-ylpiperazin-1-yl)propan-1-one oxime

The title compound was prepared using the procedure described in Example 15B except using the product from Example 7A instead of product from Example 15A. MS (DCI/NH₃) m/z 325 (M+H)⁺. ¹H NMR (300 MHz, DMSO-d₆) δ 2.33 (s, 3H), 2.50 (m, 6H), 2.92 (m, 2H), 3.45 (t, J=6 Hz, 4H), 6.62 (d-d, J=4.5 Hz, 7 Hz, 1H), 6.80 (d, J=9 Hz, 1H), 7.18 (d, J=6 Hz, 1H), 7.28 (t, J=7 Hz, 1H), 7.48 (m, 3H), 8.10 (d-d, J=3 Hz, 4.5 Hz,1H); MS (DCI/NH₃) m/z 325 (M+H)⁺. Anal. calcd for C₁₉H₂₄N₄O.0.1H₂O: C, 69.95; H, 7.48; N, 17.17. Found: C, 69.90; H, 7.62; N, 16.87.

### EXAMPLE 98

### (1E)-1-(4-chlorophenyl)-2-methoxy-3-(4-pyridin-2-ylpiperazin-1-yl)propan-1-one O-methyloxime and

### (1Z)-1-(4-chlorophenyl)-2-methoxy-3-(4-pyridin-2-ylpiperazin-1-yl)propan-1-one O-methyloxime

The title compounds were isolated as the side products of Example 95C. Z-isomer: ¹H NMR (300 MHz, DMSO-d₆) δ 2.56 (m, 5H), 2.80 (d-d, J=7 Hz, 12 Hz, 1H), 3.14 (s, 3H), 3.42 (t, J=6 Hz, 4H), 3.94 (s, 3H), 5.05 (d-d, J=4.5 Hz, 7 Hz, 1H), 6.62 (d-d, J=4.5 Hz, 7 Hz, 1H), 6.80 (d, J=7 Hz, 1H), 7.50 (m, 3H), 7.68 (d, J=9 Hz, 1H), 8.08 (m, 1H); MS (DCI/NH₃) m/z 389 (M+H)⁺. Anal. calcd for C₂₀H₂₅ClN₄O₂: C, 61.77; H, 6.48; N, 14.41. Found: C, 61.94; H, 6.45; N, 13.90.

E-isomer: ¹H NMR (300 MHz, DMSO-d₆) δ 2.30 (m, 4H), 2.40 (d-d, J=6 Hz, 12 Hz, 1H), 2.55 (m, 1H), 3.35 (s, 3H), 3.42 (m, 4H), 3.76 (s, 3H), 4.17 (t, J=7 Hz, 1H), 6.62 (d-d, J=4.5 Hz, 7 Hz, 1H), 6.80 (d, J=7 Hz, 1H), 7.34 (d, J=9 Hz, 2H), 7.50 (m, 3H), 8.10 (m, 1H); MS (DCI/NH₃) m/z 389 (M+H)⁺. Anal. calcd for C₂₀H₂₅ClN₄O₂: C, 61.77; H, 6.48; N, 14.41. Found: C, 61.65; H, 6.55; N, 13.98.

### EXAMPLE 99

### 2-{1-[(3E)-3-(4-chlorophenyl)-3-(methoxyimino)propyl]piperidin-4-yl}pyridinium N-oxide and

### 2-{1-[(3Z)-3-(4-chlorophenyl)-3-(methoxyimino)propyl]piperidin-4-yl}pyridinium N-oxide

### EXAMPLE 99A

### tert-butyl 4-{[(trifluoromethyl)sulfonyl]oxy}-3,6-dihydropyridine-1(2H)-carboxylate

Diisopropylamine (13.4 mL, 96 mmol) in THF (350 mL) at −78° C. was treated with 1.6M nBuLi in hexanes (60 mL, 96 mmol). The mixture was stirred for 5 minutes at −78° C. and treated with a solution of tert-butyl 4-oxopiperidine-1-carboxylate (16 g, 80 mmol) in THF (100 mL). The mixture was stirred for 10 minutes and treated with a solution of N-phenyltrifluoromethanesulfonimide (31.4 g, 88 mmol). The mixture was stirred at −78° C. for 30 minutes and the cooling bath was removed. After stirring for an additional 1,5 hours at room temperature, the mixture was treated with saturated NaHCO₃, diethyl ether, and 5% citric acid. The organic layer was separated, washed with 1N NaOH (4×200 mL), water (2×200 mL), brine (1×200 mL), dried over MgSO₄, filtered, and the filtrate was concentrated under reduced pressure. The residue was purified by flash chromatography (hexanes:ethyl acetate 8:2) to provide the title compound. ¹H NMR (300 MHz, DMSO-d₆) δ 1.41 (s, 9H), 2.41 (m, 2H), 3.54 (t, 2H), 3.98 (m, 2H), 6.02 (m, 1H).

### EXAMPLE 99B

### tert-butyl 3′,6′-dihydro-2,4′-bipyridine-1′(2′H)-carboxylate

The product from Example 99A (18 g, 54 mmol) in THF (200 mL) was treated with 2-pyridinylzinc bromide 0.5 molar solution in THF (124 mL, 62.5 mmol), Pd(PPh₃)₄ (625 mg) and heated at 60° C. for 90 minutes. The mixture was allowed to cool to room temperature, concentrated under reduced pressure and partitioned between ethyl acetate (300 mL) and 1N NaOH (200 mL). The mixture was filtered and the organic layer was separated, washed with brine (300 mL), dried over anhydrous MgSO₄, filtered, and the filtrate was concentrated under reduced pressure. The residue was purified by chromatography (hexanes:ethyl acetate 6:4) to provide the title compound. ¹H NMR (300 MHz, DMSO-d₆) δ 1.43 (s, 9H), 2.56 (m, 2H), 3.54 (t, 2H), 4.04 (m, 2H), 6.08 (m, 1H), 7.25 (dd, 1H), 7.56 (d, J=9 Hz, 1H), 7.77 (m, 1H), 8.54 (m, 1H); MS (DCI—NH₃) m/z 259 (M+H)⁺, 277 (M+NH₄)⁺

### EXAMPLE 99C

### tert-butyl 4-pyridin-2-ylpiperidine-1-carboxylate

The product from Example 99B (9.0 g, 35 mmol) ) was treated with 10%Pd/C (900 mg) under a hydrogen atmosphere (60 psi) at room temperature for 1.5 hours to provide the title compound. 1H NMR (300 MHz, DMSO-d₆) δ 1.41 (s, 9H), 1.58 (m, 2H), 1.81 (m, 2H), 2.85 (m, 3H), 4.06 (m, 2H), 7.20 (dd, 1H), 7.28 (d, J=9 Hz, 1H), 7.70 (m, 1H), 8.48 (m, 1H).

### EXAMPLE 99D

### 2-[1-(tert-butoxycarbonylypiperidin-4-yl]pyridinium N-oxide

The product from Example 99C (8.9 g, 33.9 mmol) in dichloromethane (30 mL) was cooled to 0° C. and treated with m-chloroperbenzoic acid (77% purity) (10.5 g, 61.1 mmol). After stirring at 0° C. for 30 minutes, the mixture was allowed to warm to room temperature and stir for 2 hours. The mixture was diluted with CH₂Cl₂ (50 mL) and washed with saturated NaHCO₃, brine, dried over MgSO₄, filtered, and the filtrate was concentrated under reduced pressure. The residue was triturated with 5% CH₂Cl₂ in hexanes to provide the title compound. ¹H NMR (300 MHz, DMSO-d₆) δ 1.41 (s, 9H), 1.42 (m, 2H), 1.90 (m, 2H), 2.83 (m, 2H), 3.45 (m, 1H), 4.09 (m, 2H), 7.30 (m, 2H), 7.40 (m, 1H), 8.26 (m, 1H).

### EXAMPLE 99E

### 2-piperidin-4-ylpyridinium N-oxide

The product from Example 99D (6.57 g, 23.8 mmol) in ethyl acetate (150 mL) was cooled to −78° C. and HCl gas was bubbled through the mixture for 15 minutes. The mixture was allowed to warm to room temperature and was filtered. The filter cake was washed with ethyl acetate and dried under reduced pressure to provide the title compound. ¹H NMR (300 MHz, DMSO-d₆) δ 1.82 (m, 2H), 2.10 (m, 2H), 3.06 (m, 2H), 3.36 (m, 2H), 3.58 (m, 1H), 7.45 (m, 3H), 8.39 (d, J=9 Hz, 1H), 9.04 (bs, 1H); MS (DCI—NH₃) m/z 179 (M+H)⁺, 163 (M+H-16)⁺.

### EXAMPLE 99F

### 2-{1-[3-(4-chlorophenyl)-3-oxopropyl]piperidin-4-yl}pyridinium N-oxide

3,4′-Dichloropropiophenone (1.00 g, 4.92 mmol), the product from Example 99E (2.10 g, 9.84 mmol), and potassium carbonate (2.03 g, 14.76 mmol) were combined in DMF (15 mL) and heated at 80° C. for 16 hours. The mixture was concentrated under reduced pressure and the residue purified by chromatography (CH₂Cl₂:MeOH 4:1) to provide the title compound. ¹H NMR (300 MHz, DMSO-d₆) δ 1.50 (dq, J=4 Hz, 12 Hz, 2H), 1.88 (d, J=12 Hz, 2H), 2.09 (t, J=11 Hz, 2H), 2.72 (t, J=7.1 Hz, 2H), 3.00 (d, J=11.5 Hz, 2H), 3.21 (t, J=7.1 Hz, 3H), 7.28 (m, 2H), 7.38 (dd, J=2 Hz, 7.1 Hz, 11H), 7.60 (d, J=9 Hz, 2H), 8.00 (d, J=9 Hz, 2H), 8.25 (m, 1H); MS (DCI/NH₃) m/z 345 (M+H)⁺.

### EXAMPLE 99G

### 2-{1-[3-(4-chlorophenyl)-3-(methoxyimino)propyl]piperidin-4-yl]pyridinium N-oxide

The product from Example 99F, (340 mg, 0.99 mmol) and O-methylhydroxylamine hydrochloride (412 mg, 4.93 mmol) were combined in pyridine (25 mL) and stirred at ambient temperature for 12 hours. The mixture was concentrated under reduced pressure and the residue was partitioned between saturated NaHCO₃ and ethyl acetate. The organic phase was washed with brine, dried over magnesium sulfate, filtered, and the filtrate was concentrated under reduced pressure. The residue was purified by column chromatography (CH₂Cl₂:MeOH 19:1) to provide the title compounds.

E-isomer: ¹H NMR (300 MHz, DMSO-d₆) δ 1.50 (dq, J=3 Hz, 12 Hz, 2H), 1.88 (d, J=12 Hz, 2H), 2.08 (t, J=11 Hz, 2H), 2.45 (d, J=7 Hz, 1H), 2.93 (m, 5H), 3.21 (td, J=3 Hz, 12 Hz, 1H), 3.92 (s, 3H), 7.28 (m, 2H), 7.38 (m, 1H), 7.48 (d, J=9 Hz, 2H), 7.68 (d, J=9 Hz, 2H); 8.24 (m, 1H); MS (DCI/NH₃) m/z 374 (M+H)⁺. Anal. calcd. for C₂₀H₂₄ClN₃O₂.C₄H₄O₄ (maleate salt): C, 58.83; H, 5.76; N, 8.58. Found: C, 59.05; H, 5.62; N, 8.55.

Z-isomer: ¹H NMR (300 MHz, DMSO-d₆) δ 1.50 (dq, J=4 Hz, 12 Hz, 2H), 1.88 (d, J=12 Hz, 2H), 2.00 (t, J=11 Hz, 2H), 2.37 (t, J=7.5 Hz, 2H), 2.68 (t, J=7.5 Hz, 2H), 3.21 (m, 1H), 3.72 (s, 3H), 7.30 (m, 2H), 7.38 (m, 2H), 7.45 (d, J=4.5 Hz, 2H), 7.49 (m, 1H); 8.21 (m, 1H); MS (DCI/NH₃) m/z 374 (M+H)⁺. Anal. calcd. for C₂₀H₂₄ClN₃O₂.C₄H₄O₄ (maleate salt): C, 58.83; H, 5.76; N, 8.58. Found: C, 59.01; H, 5.89; N, 8.74.

### EXAMPLE 100

### 2-{1-[(2E)-2-(4-fluorophenyl)-2-(methoxyimino)ethyl]piperidin-4-yl}pyridinium N-oxide and

### 2-{1-[(2Z)-2-(4-fluorophenyl)-2-(methoxyimino)ethyl]piperidin-4-yl}pyridinium N-oxide

### EXAMPLE 100A

### 2-{1-[2-(4-fluorophenyl)-2-oxoethyl]piperidin-4-yl}pyridinium N-oxide

The title compound was prepared using the procedure described in Example 99F except using 2-chloro-4′-fluoroacetophenone instead of 3,4′-dichloropropiophenone. ¹H NMR (300 MHz, DMSO-d₆) δ 1.50 (dq, J=3 Hz, 9 Hz, 2H), 1.88 (d, J=12 Hz, 2H), 2.27 (t, J=12 Hz, 2H), 3.0 (d, J=12 Hz, 2H), 3.25 (m, 1H), 3.84 (s, 2H), 7.30 (m, 3H), 7.95 (s, 2H), 8.12 (dd, J=6 Hz, 9 Hz, 2H), 8.24 (m, 1H); MS (DCI/NH₃) m/z 315 (M+H)⁺.

### EXAMPLE 100B

### 2-{1-[(2E)-2-(4-fluorophenyl)-2-(methoxyimino)ethyl]piperidin-4-yl}pyridinium N-oxide and

### 2-{1-[(2Z)-2-(4-fluorophenyl)-2-(methoxyimino)ethyl]piperidin-4-yl}pyridinium N-oxide

The title compounds were prepared using the procedure described in Example 99G except using the product from Example 100A instead of the product from Example 99F. ¹H NMR (300 MHz, DMSO-d₆) δ 1.50 (dq, J=3 Hz, 12 Hz, 2H), 1.85 (d, J=12 Hz, 2H), 2.15 (t, J=11 Hz, 2H), 2.91 (d, J=7 Hz, 2H), 3.21 (m, 1H), 3.65 (s, 2H), 3.91 (s, 3H), 7.24 (m, 2H), 7.34 (m, 1H), 7.80 (dd, J=6 Hz, 9 Hz, 2H); 8.22 (t, J=3 Hz, 1H); MS (DCI/NH₃) m/z 344 (M+H)⁺; Anal. calcd. for C₁₉H₂₂FN₃O₂: C, 66.45; H, 6.46; N, 12.24. Found: C, 66.25, H, 6.57, N, 12.17.

### In Vitro Data

### Functional Activity of D₄

Efficacies and potencies of compounds of the present invention at the human D₄ receptor were determined using a stable cell line containing the human D_{4,4} receptor and a chimeric G protein in HEK-293 cells. This cell line allows a robust calcium signal detectable using a calcium fluorescent dye and a fluorescent imaging plate reader (FLIPR) (Coward et al., Anal. Biochem. Vol. 270, pages 242-248, 1999). Cells were plated (20000/well) into 96 well dishes and cultured for 48 hours. Media is removed, Fluo-4 dye added and cells incubated 1 hour at room temperature. Cells are washed with phosphate buffered saline to remove excess dye and compounds to be tested added to the wells and signal measured in FLIPR. Percent efficacy is the maximum response produced by the compound in relation to the maximum effect of 10 μM dopamine. Representative compounds of the present invention exhibited percent efficacies in the range of about 5% to about 93%. The EC₅₀ is the effective concentration of the compound that causes 50% of the compound's maximum response. Representative compounds of the present invention exhibited EC₅₀s in the range of about 2 nM to about 1800 nM.

The in vitro data demonstrates that compounds of the present invention illicit the same response from dopamine D₄ receptors as does dopamine.

### In Vivo Data

### Rat Penile Erection Model

Wistar rats were used as a primary animal model to study penile erection in vivo. All experiments were carried out between 9:00 AM and 3:00 PM in a diffusely illuminated testing room with a red light. Animals (n=8-30) were weighed and allowed to adapt to the testing room for 60 minutes before the beginning of experiments. Rats were placed individually in a transparent cage (20×30×30 cm) after drug injection. The number of penile erections were recorded by direct observation for a period of 60 minutes after drug dosing, and the number of animals exhibiting 1 or more erections is expressed as incidence (%).

Tested compounds were as effective as the most efficacious dose of 0.1 μmol/kg of apomorphine, with maximal effective dose ranging from 0.03-1.0 μmol/kg. The maximum % of incidence of erections in rat was 85%.

Compounds of the present invention can be used in combination with phosphodiesterase 5 inhibitors including, but not limited to, sildenafil or vardenafil as a method of treating sexual dysfunction in a mammal.

Compounds of the present invention can be used in combination with an adrenergic receptor antagonist including, but not limited to, terazosin, prazosin or tamsulosin as method of treating sexual dysfunction in a mammal.

Compounds of the present invention can be used in combination with a dopamine agonist including, but not limited to, apomorphine as a method of treating sexual dysfunction in a mammal.

Compounds of the present invention illicit the same response from dopamine D₄ receptors as does dopamine and therefore compounds of the present invention are useful for the treatment of male sexual dysfunction, female sexual dysfunction, attention deficit hyperactivity disorder, Alzheimer's disease, drug abuse, Parkinson's disease, anxiety, schizophrenia, mood disorders and depression, as described in: N. J. Hrib, Drugs of the future Vol. 25, pages 587-611 (2000); M. Melis and A. Argiolas, Neuroscience and Biobehavioral Reviews Vol. 19, pages19-38 (1995); and C. Missale, S. R. Nash, S. Robinson, M. Jabber and M. Caron, Physiological Reviews Vol. 78, pages 189-225 (1998).

Compounds of the present invention illicit the same response from dopamine D₄ receptors as does dopamine and therefore compounds of the present invention are useful for the treatment of cardiovascular disorders. Dopamine and dopaminergic agents have been reported to exert pharmacologically significant cardiovascular effects on blood pressure and heart rate and are useful in the treatment of cardiovascular disorders, as described in: Chen F F, and Lin M T, J. Pharmacol. Exp. Therap. Vol. 214, pages 427-432 (1980); and it has been reported that primate data support the potential clinical utility of dopamine receptor agonists in treating cardiovascular disease, as described in: Hahn, R A and MacDonald B R, J. Pharmacol. Exp. Therap. Vol 229, pages 132-138 (1984).

Compounds of the present invention illicit the same response from dopamine D₄ receptors as does dopamine and therefore compounds of the present invention are useful for the treatment of inflammation. Dopaminergic agents can exert anti-inflammatory effects and are useful for the treatment of diseases where inflammation plays a deleterious role, as described in: Bendele A M, Spaethe S M, Benslay D N, and Bryant H U, J. Pharmacol. Exp. Therap. Vol 259, pages 169-175 (1991). Dopaminergic agents can also be of utility in the treatment of cancers, as described in: Lissoni P, Mandala M, Giani L, Malugani F, Secondino S, Zonato S, Rocco F, Gardani G, Neuroendocrinology Letters Vol. 21 pages 405-408 (2000).

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

Dosage forms for topical administration of a compound of the present invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated.

When used in the above or other treatments, a therapeutically effective amount of one of the compounds of the present invention can be employed in pure form or, where such forms exist, in pharmaceutically acceptable salt, ester, amide, or prodrug form. Alternatively, the compound can be administered as a pharmaceutical composition containing the compound of interest in combination with one or more pharmaceutically acceptable carriers. The phrase “therapeutically effective amount” of the compound of the present invention means a sufficient amount of the compound to treat disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of the present invention administered to a mammal, and particularly a human, may range from about 0.001 to about 30 mg/kg/day. For purposes of oral administration, more preferable doses can be in the range of from 0.01 to about 10 mg/kg/day. If desired, the effective daily dose can be divided into multiple doses for purposes of administration; consequently, single dose compositions may contain such amounts or submultiples thereof to make up the daily dose.

The present invention also provides pharmaceutical compositions that comprise compounds of the present invention formulated together with one or more non-toxic pharmaceutically acceptable carriers. The pharmaceutical compositions can be specially formulated for oral administration in solid or liquid form, for parenteral injection or for rectal administration.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally” as used herein, refers to modes of administration, which include intravenous, intramuscular, intraperitoneal, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Besides inert diluents, the oral compositions may also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The present invention contemplates pharmaceutically active compounds either chemically synthesized or formed by in vivo biotransformation to compounds of formula (I-III)).

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The term “pharmaceutically acceptable salts and prodrugs” as used herein, refers to carboxylate salts, amino acid addition salts, zwitterions, and prodrugs of compounds of formula (I-III)) which are within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. The term “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, sulfate, bis(tartrate), tartrate, (L) tartrate, bis((L) tartrate), (D) tartrate, bis((L) tartrate), (DL) tartrate, bis((DL) tartrate), meso-tartrate, bis(meso tartrate), thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulphuric acid and phosphoric acid and such organic acids as maleic acid, fumaric acid, succinic acid and citric acid.

The term “pharmaceutically acceptable prodrug” or “prodrug” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I-III), for example, by hydrolysis in blood.

1. A compound of formula (I) 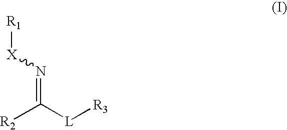
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 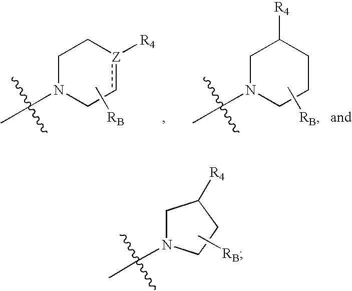
R₄ is heteroaryl;
L is C₁-C₂ alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C, CH, and N; and
— is absent or a single bond provided that when — is a single bond then Z is C.

2. The compound according to claim 1 wherein R₃ is

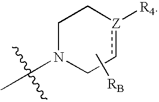

3. The compound according to claim 2 wherein
X is O;
R₂ is aryl;
Z is N;
— is absent; and
R₄ is heteroaryl.

4. The compound according to claim 2 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

5. The compound according to claim 4 selected from the group consisting of
(1E)-1-(3-chlorophenyl)-3-(4-pyridin-2-yl-piperazin-1-yl)propan-1-one O-methyloxime;
(1Z)-1-(3-chlorophenyl)-3-(4-pyridin-2-ylpiperazin-1-yl)propan-1-one O-methyloxime;
(1E)-1-(4-chlorophenyl)-3-(4-pyridin-2-yl-piperazin-1-yl)propan-1-one O-methyloxime;
(1Z)-1-(4-chlorophenyl)-3-(4-pyridin-2-ylpiperazin-1-yl)propan-1-one O-methyloxime;
(1E)-1-(4-fluorophenyl)-2-(4-pyridin-2-ylpiperazin-1-yl)ethanone O-methyloxime;
(1Z)-1-(4-fluorophenyl)-2-(4-pyridin-2-ylpiperazin-1-yl)ethanone O-methyloxime;
(1E)-1-(4-chlorophenyl)-2-(4-pyridin-2-ylpiperazin-1-yl)ethanone O-methyloxime;
(1Z)-1-(4-chlorophenyl)-2-(4-pyridin-2-ylpiperazin-1-yl)ethanone O-methyloxime;
(1E)-1-(3,4-dimethylphenyl)-3-(4-pyridin-2-ylpiperazin-1-yl)propan-1-one O-methyloxime;
(1Z)-1-(3,4-dimethylphenyl)-3-(4-pyridin-2-ylpiperazin-1-yl)propan-1-one O-methyloxime;
(1E)-1-(3-chloro-4-fluorophenyl)-3-(4-pyridin-2-ylpiperazin-1-yl)propan-1-one O-methyloxime;
(1Z)-1-(3-chloro-4-fluorophenyl)-3-(4-pyridin-2-ylpiperazin-1-yl)propan-1-one O-methyloxime;
(1E)-1-(3-methylphenyl)-3-(4-pyridin-2-ylpiperazin-1-yl)propan-1-one O-methyloxime;
(1Z)-1-(3-methylphenyl)-3-(4-pyridin-2-ylpiperazin-1-yl)propan-1-one O-methyloxime;
(1E)-1-(4-fluorophenyl)-3-(4-pyridin-2-ylpiperazin-1-yl)propan-1-one O-methyloxime;
(1Z)-1-(4-fluorophenyl)-3-(4-pyridin-2-ylpiperazin-1-yl)propan-1-one O-methyloxime;
(1E)-1-(3,4-dichlorophenyl)-2-(4-pyridin-2-ylpiperazin-1-yl)ethanone O-methyloxime;
(1Z)-1-(3,4-dichlorophenyl)-2-(4-pyridin-2-ylpiperazin-1-yl)ethanone O-methyloxime;
(1E)-1-(2-methylphenyl)-3-(4-pyridin-2-ylpiperazin-1-yl)propan-1-one O-ethyloxime;
(1Z)-1-(2-methylphenyl)-3-(4-pyridin-2-ylpiperazin-1-yl)propan-1-one O-ethyloxime;
(1E)-1-(2-methylphenyl)-3-(4-pyridin-2-ylpiperazin-1-yl)propan-1-one O-methyloxime;
(1Z)-1-(2-methylphenyl)-3-(4-pyridin-2-ylpiperazin-1-yl)propan-1-one O-methyloxime;
3-[(1E)-N-methoxy-3-(4-pyridin-2-ylpiperazin-1-yl)propanimidoyl]benzonitrile
3-[(1Z)-N-methoxy-3-(4-pyridin-2-ylpiperazin-1-yl)propanimidoyl]benzonitrile
(1E)-1-(2,4-dichlorophenyl)-2-(4-pyridin-2-ylpiperazin-1-yl)ethanone O-methyloxime;
(1Z)-1-(2,4-dichlorophenyl)-2-(4-pyridin-2-ylpiperazin-1-yl)ethanone O-methyloxime;
(1E)-1-phenyl-3-(4-pyridin-2-ylpiperazin-1-yl)propan-1-one oxime;
(1Z)-1-phenyl-3-(4-pyridin-2-ylpiperazin-1-yl)propan-1-one oxime;
1,5-diphenyl-2-[(4-pyridin-2-ylpiperazin-1-yl)methyl]pentane-1,5-dione dioxime;
(1E)-1-phenyl-3-(4-pyrimidin-2-ylpiperazin-1-yl)propan-1-one oxime;
(1Z)-1-phenyl-3-(4-pyrimidin-2-ylpiperazin-1-yl)propan-1-one oxime;
1,5-diphenyl-2-[(4-pyrimidin-2-ylpiperazin-1-yl)methyl]pentane-1,5-dione dioxime;
1-(4-fluorophenyl)-3-(4-pyridin-2-ylpiperazin-1-yl)propan-1-one oxime;
(1E)-1-(4-chlorophenyl)-3-(4-pyridin-2-ylpiperazin-1-yl)propan-1-one oxime;
(1E)-1-phenyl-3-(4-pyridin-2-ylpiperazin-1-yl)propan-1-one O-ethyloxime;
(1Z)-1-phenyl-3-(4-pyridin-2-ylpiperazin-1-yl)propan-1-one O-ethyloxime;
(1E)-1-phenyl-3-(4-pyridin-2-ylpiperazin-1-yl)propan-1-one O-methyloxime;
(1Z)-1-phenyl-3-(4-pyridin-2-ylpiperazin-1-yl)propan-1-one O-methyloxime;
(1E)-1-phenyl-3-(4-pyridin-2-ylpiperazin-1-yl)propan-1-one O-propyloxime;
(1Z)-1-phenyl-3-(4-pyridin-2-ylpiperazin-1-yl)propan-1-one O-propyloxime;
(1E)-1-phenyl-3-(4-pyridin-2-ylpiperazin-1-yl)propan-1-one O-allyloxime;
(1Z)-1-phenyl-3-(4-pyridin-2-ylpiperazin-1-yl)propan-1-one O-allyloxime;
(1E)-1-(3,5-difluorophenyl)-3-(4-pyridin-2-ylpiperazin-1-yl)propan-1-one O-methyloxime;
(1Z)-1-(3,5-difluorophenyl)-3-(4-pyridin-2-ylpiperazin-1-yl)propan-1-one O-methyloxime;
({[1-phenyl-3-(4-pyridin-2-ylpiperazin-1-yl)propylidene]amino}oxy)acetonitrile;
1-phenyl-3-(4-pyridin-2-ylpiperazin-1-yl)propan-1-one O-butyloxime;
(1E)-1-phenyl-3-(4-pyridin-2-ylpiperazin-1-yl)propan-1-one O-isopropyloxime;
(1E)-1-(3,5-dimethylphenyl)-3-(4-pyridin-2-ylpiperazin-1-yl)propan-1-one O-methyloxime;
(1Z)-1-(3,5-dimethylphenyl)-3-(4-pyridin-2-ylpiperazin-1-yl)propan-1-one O-methyloxime;
(1E)-1-(4-chloro-3-methylphenyl)-3-(4-pyridin-2-ylpiperazin-1-yl)propan-1-one O-methyloxime;
(1Z)-1-(4-chloro-3-methylphenyl)-3-(4-pyridin-2-ylpiperazin-1-yl)propan-1-one O-methyloxime;
(1E)-1-(2-naphthyl)-2-(4-pyridin-2-ylpiperazin-1-yl)ethanone O-methyloxime;
(1Z)-1-(2-naphthyl)-2-(4-pyridin-2-ylpiperazin-1-yl)ethanone O-methyloxime;
(1E)-1-(3-methylphenyl)-3-(4-pyridin-2-ylpiperazin-1-yl)propan-1-one O-ethyloxime;
(1Z)-1-(3-methylphenyl)-3-(4-pyridin-2-ylpiperazin-1-yl)propan-1-one O-ethyloxime;
1-(4-fluorophenyl)-3-(4-pyridin-2-ylpiperazin-1-yl)propan-1-one O-(2,2,2-trifluoroethyl)oxime;
1-(4-chlorophenyl)-3-(methoxyamino)-2-[(4-pyridin-2-ylpiperazin-1-yl)methyl]propan-1-one O-methyloxime;
1-(4-chlorophenyl)-3-isopropoxy-2-[(4-pyridin-2-ylpiperazin-1-yl)methyl]propan-1-one O-methyloxime;
1-(4-chlorophenyl)-2-methyl-3-(4-pyridin-2-ylpiperazin-1-yl)propan-1-one O-methyloxime;
(1E)-1-(3,4-dichlorophenyl)-3-(4-pyridin-2-ylpiperazin-1-yl)propan-1-one O-methyloxime;
(1Z)-1-(3,4-dichlorophenyl)-3-(4-pyridin-2-ylpiperazin-1-yl)propan-1-one O-methyloxime;
(1E)-1-(2-chlorophenyl)-3-(4-pyridin-2-ylpiperazin-1-yl)propan-1-one O-methyloxime;
(1Z)-1-(2-chlorophenyl)-3-(4-pyridin-2-ylpiperazin-1-yl)propan-1-one O-methyloxime;
(1E)-1-(2,4-dichlorophenyl)-3-(4-pyridin-2-ylpiperazin-1-yl)propan-1-one O-methyloxime;
(1Z)-1-(2,4-dichlorophenyl)-3-(4-pyridin-2-ylpiperazin-1-yl)propan-1-one O-methyloxime;
(1E)-1-(4-bromophenyl)-3-(4-pyridin-2-ylpiperazin-1-yl)propan-1-one O-methyloxime;
(1Z)-1-(4-bromophenyl)-3-(4-pyridin-2-ylpiperazin-1-yl)propan-1-one O-methyloxime;
(1E)-1-(3-fluorophenyl)-3-(4-pyridin-2-ylpiperazin-1-yl)propan-1-one O-methyloxime;
(1Z)-1-(3-fluorophenyl)-3-(4-pyridin-2-ylpiperazin-1-yl)propan-1-one O-methyloxime;
(1E)-1-(4-fluorophenyl)-2-(4-pyrimidin-2-ylpiperazin-1-yl)ethanone oxime;
(1Z)-1-(4-fluorophenyl)-2-(4-pyrimidin-2-ylpiperazin-1-yl)ethanone oxime;
(1E)-1-(4-fluorophenyl)-2-(4-pyridin-2-ylpiperazin-1-yl)ethanone oxime;
(1Z)-1-(4-fluorophenyl)-2-(4-pyridin-2-ylpiperazin-1-yl)ethanone oxime;
2-{4-[(3E)-3-(hydroxyimino)-3-phenylpropyl]piperazin-1-yl}nicotinonitrile;
2-{4-[(3Z)-3-(hydroxyimino)-3-phenylpropyl]piperazin-1-yl}nicotinonitrile;
1-phenyl-3-[4-(1,3-thiazol-2-yl)piperazin-1-yl]propan-1-one oxime;
1-phenyl-3-(4-pyrimidin-2-ylpiperazin-1-yl)propan-1-one O-ethyloxime;
1-phenyl-3-[4-(1,3-thiazol-2-yl)piperazin-1-yl]propan-1-one O-ethyloxime;
3-[4-(3-methylpyridin-2-yl)piperazin-1-yl]-1-phenylpropan-1-one O-ethyloxime;
2-{4-[3-(ethoxyimino)-3-phenylpropyl]piperazin-1-yl}nicotinonitrile;
2-{4-[3-(ethoxyimino)-3-(3-methylphenyl)propyl]piperazin-1-yl}nicotinonitrile;
(1E)-1-(4-fluorophenyl)-2-[(2S)-2-methyl-4-pyridin-2-ylpiperazin-1-yl]ethanone O-methyloxime;
(1Z)-1-(4-fluorophenyl)-2-[(2S)-2-methyl-4-pyridin-2-ylpiperazin-1-yl]ethanone O-methyloxime;
(1E)-1-(4-chlorophenyl)-3-[(2S)-2-methyl-4-pyridin-2-ylpiperazin-1-yl]propan-1-one O-methyloxime;
(1Z)-1-(4-chlorophenyl)-3-[(2S)-2-methyl-4-pyridin-2-ylpiperazin-1-yl]propan-1-one O-methyloxime;
1-(4-chlorophenyl)-3-(4-pyridin-2-ylpiperazin-1-yl)propan-1-one O-(2-hydroxyethyl)oxime;
(1E)-1-(4-chlorophenyl)-3-[4-(5-hydroxypyridin-2-yl)piperazin-1-yl]propan-1-one O-methyloxime;
(1Z)-1-(4-chlorophenyl)-3-[4-(5-hydroxypyridin-2-yl)piperazin-1-yl]propan-1-one O-methyloxime;
1-(4-fluorophenyl)-2-[4-(5-hydroxypyridin-2-yl)piperazin-1-yl]ethanone O-methyloxime;
(1E)-1-(4-chlorophenyl)-2-hydroxy-3-(4-pyridin-2-ylpiperazin-1-yl)propan-1-one O-methyloxime;
(1Z)-1-(4-chlorophenyl)-2-hydroxy-3-(4-pyridin-2-ylpiperazin-1-yl)propan-1-one O-methyloxime;
(1E)-1-(4-chlorophenyl)-3-(4-pyrimidin-2-ylpiperazin-1-yl)propan-1-one O-methyloxime;
(1Z)-1-(4-chlorophenyl)-3-(4-pyrimidin-2-ylpiperazin-1-yl)propan-1-one O-methyloxime;
(1E)-1-(4-chlorophenyl)-2-methoxy-3-(4-pyridin-2-ylpiperazin-1-yl)propan-1-one O-methyloxime; and
(1Z)-1-(4-chlorophenyl)-2-methoxy-3-(4-pyridin-2-ylpiperazin-1-yl)propan-1-one O-methyloxime.

6. The compound according to claim 2 wherein
X is O;
R₂ is arylalkyl;
Z is N;
— is absent; and
R₄ is heteroaryl.

7. The compound according to claim 2 wherein
X is O;
R₂ is arylalkyl wherein the arylalkyl is benzyl;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

8. The compound according to claim 7 selected from the group consisting of
(2E)-1-phenyl-3-(4-pyridin-2-ylpiperazin-1-yl)acetone O-methyloxime; and
(2Z)-1-phenyl-3-(4-pyridin-2-ylpiperazin-1-yl)acetone O-methyloxime.

9. The compound according to claim 2 wherein
X is O;
R₂ is heteroaryl;
Z is N;
— is absent; and
R₄ is heteroaryl.

10. The compound according to claim 2 wherein
X is O;
R₂ is heteroaryl wherein the heteroaryl is pyridin-3-yl;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

11. The compound according to claim 4 selected from the group consisting of
(1E)-1-pyridin-3-yl-3-(4-pyridin-2-ylpiperazin-1-yl)propan-1-one O-methyloxime; and
(1Z)-1-pyridin-3-yl-3-(4-pyridin-2-ylpiperazin-1-yl)propan-1-one O-methyloxime.

12. The compound according to claim 2 wherein
X is O;
R₂ is aryl;
Z is C;
— is a single bond; and
R₄ is heteroaryl.

13. The compound according to claim 2 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is C;
— is a single bond; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

14. The compound according to claim 13 selected from the group consisting of
1-(4-fluorophenyl)-3-[4-(1,3-thiazol-2-yl)-3,6-dihydropyridin-1(2H)-yl]propan-1-one O-methyloxime;
(1E)-1-(4-chlorophenyl)-2-[4-(1,3-thiazol-2-yl)-3,6-dihydropyridin-1(2H)-yl]ethanone O-methyloxime;
(1Z)-1-(4-chlorophenyl)-2-[4-(1,3-thiazol-2-yl)-3,6-dihydropyridin-1(2H)-yl]ethanone O-methyloxime;
(1E)-1-(4-chlorophenyl)-3-(3-methyl-3′,6!-dihydro-2,4′-bipyridin-1′(2′H)-yl)propan-1-one O-methyloxime; and
(1Z)-1-(4-chlorophenyl)-3-(3-methyl-3′,6′-dihydro-2,4′-bipyridin-1′(2′H)-yl)propan-1-one O-methyloxime.

15. The compound according to claim 2 wherein
X is O;
R₂ is aryl;
Z is CH;
— is absent; and
R₄ is heteroaryl.

16. The compound according to claim 2 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is CH;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

17. The compound according to claim 16 selected from the group consisting of
(1E)-1-(4-chlorophenyl)-3-(4-pyridin-2-ylpiperidin-1-yl)propan-1-one O-methyloxime;
(1Z)-1-(4-chlorophenyl)-3-(4-pyridin-2-ylpiperidin-1-yl)propan-1-one O-methyloxime;
2-{1-[(3E)-3-(4-chlorophenyl)-3-(methoxyimino)propyl]piperidin-4-yl}pyridinium N-oxide;
2-{1-[(3Z)-3-(4-chlorophenyl)-3-(methoxyimino)propyl]piperidin-4-yl}pyridinium N-oxide;
2-{1-[(2E)-2-(4-fluorophenyl)-2-(methoxyimino)ethyl]piperidin-4-yl}pyridinium N-oxide; and
2-{1-[(2Z)-2-(4-fluorophenyl)-2-(methoxyimino)ethyl]piperidin-4-yl}pyridinium N-oxide.

18. The compound according to claim 2 wherein
X is NR_{A};
R₂ is aryl;
Z is N;
— is absent; and
R₄ is heteroaryl.

19. The compound according to claim 2 wherein
X is NR_{A};
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

20. The compound according to claim 19 that is 1-(4-fluorophenyl)-2-(4-pyridin-2-ylpiperazin-1-yl)ethanone methylhydrazone.

21. The compound according to claim 2 wherein
X is NR_{A};
R₂ is aryl;
Z is CH;
— is absent; and
R₄ is heteroaryl.

22. The compound according to claim 2 wherein
X is NR_{A};
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is CH;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

23. The compound according to claim 22 that is 1-(4-fluorophenyl)-2-(4-pyridin-2-ylpiperidin-1-yl)ethanone methylhydrazone.

24. The compound according to claim 1 wherein R₃ is

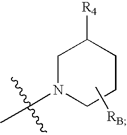

25. The compound according to claim 24 wherein
R₂ is aryl; and
R₄ is heteroaryl.

26. The compound according to claim 24 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

27. The compound according to claim 26 selected from the group consisting of
1-(4-fluorophenyl)-3-[3-(1,3-thiazol-2-yl)piperidin-1-yl]propan-1-one O-methyloxime;
1-(4-fluorophenyl)-3-[3-(1,3-thiazol-2-yl)piperidin-1-yl]propan-1-one O-ethyloxime;
1-(4-fluorophenyl)-3-(3-pyridin-2-ylpiperidin-1-yl)propan-1-one O-methyloxime;
1-(4-chlorophenyl)-3-(3-pyridin-2-ylpiperidin-1-yl)propan-1-one O-methyloxime;
(1E)-1-(4-chlorophenyl)-3-[3-(1,3-thiazol-2-yl)piperidin-1-yl]propan-1-one O-methyloxime;
(1Z)-1-(4-chlorophenyl)-3-[3-(1,3-thiazol-2-yl)piperidin-1-yl]propan-1-one O-methyloxime;
2-{1-[2-(4-fluorophenyl)-2-(methoxyimino)ethyl]piperidin-3-ylpyridinium N-oxide; and
2-{1-[3-(4-fluorophenyl)-3-(methoxyimino)propyl]piperidin-3-yl}pyridinium N-oxide.

28. The compound according to claim 1 wherein R₃ is

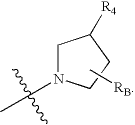

29. The compound according to claim 28 wherein
R₂ is aryl; and
R₄ is heteroaryl.

30. The compound according to claim 28 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

31. The compound according to claim 30 selected from the group consisting of
(1E)-1-(4-fluorophenyl)-3-(3-pyrazin-2-ylpyrrolidin-1-yl)propan-1-one O-methyloxime;
(1Z)-1-(4-fluorophenyl)-3-(3-pyrazin-2-ylpyrrolidin-1-yl)propan-1-one O-methyloxime;
(1E)-1-(4-fluorophenyl)-2-(3-pyrazin-2-ylpyrrolidin-1-yl)ethanone O-methyloxime;
(1Z)-1-(4-fluorophenyl)-2-(3-pyrazin-2-ylpyrrolidin-1-yl)ethanone O-methyloxime;
(1E)-1-(4-fluorophenyl)-3-(3-pyrazin-2-ylpyrrolidin-1-yl)propan-1-one oxime;
(1Z)-1-(4-fluorophenyl)-3-(3-pyrazin-2-ylpyrrolidin-1-yl)propan-1-one oxime; and
(1Z)-1-(4-fluorophenyl)-2-(3-pyrazin-2-ylpyrrolidin-1-yl)ethanone oxime.

32. A pharmaceutical composition comprising a therapeutically effective amount of a compound of formula (I) in combination with a pharmaceutically acceptable carrier.

33. A method of treating sexual dysfunction in a mammal comprising administering to the mammal a therapeutically effective amount of a compound of formula (I) or a pharmaceutically acceptable salt or prodrug thereof in combination with a pharmaceutically acceptable carrier.

34. A method of treating sexual dysfunction in a mammal comprising administering to the mammal a therapeutically effective amount of a compound of formula (I) or a pharmaceutically acceptable salt or prodrug thereof in combination with a phosphodiesterase 5 inhibitor.

35. A method of treating sexual dysfunction in a mammal comprising administering to the mammal a therapeutically effective amount of a compound of formula (I) or a pharmaceutically acceptable salt or prodrug thereof in combination with an adrenergic receptor antagonist.

36. A method of treating sexual dysfunction in a mammal comprising administering to the mammal a therapeutically effective amount of a compound of formula (I) or a pharmaceutically acceptable salt or prodrug thereof in combination with a dopamine agonist.

37. A method of treating male erectile dysfunction in a mammal comprising administering to the mammal in need of such treatment a therapeutically effective amount of a compound of formula (I) or a pharmaceutically acceptable salt or prodrug thereof.

38. A method of treating female sexual dysfunction in a mammal comprising administering to the mammal in need of such treatment a therapeutically effective amount of a compound of formula (I) or a pharmaceutically acceptable salt or prodrug thereof.

39. A method of treating cardiovascular disorders, inflammatory disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (I) or a pharmaceutically acceptable salt or prodrug thereof.

40. A method of treating sexual dysfunction in a mammal comprising administering to the mammal a therapeutically effective amount of a compound of formula (Ia) 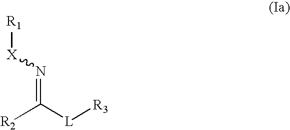
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 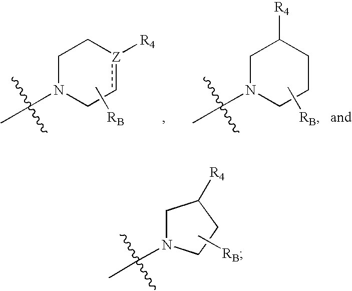
R₄ is heteroaryl;
L is alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C, CH, and N; and
— is absent or a single bond provided that when — is a single bond then Z is C;
or a pharmaceutically acceptable salt or prodrug thereof in combination with a pharmaceutically acceptable carrier.

41. The method according to claim 40 wherein R₃ is

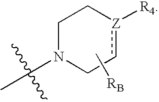

42. The method according to claim 41 wherein
X is O;
R₂ is aryl;
Z is N;
— is absent; and
R₄ is heteroaryl.

43. The method according to claim 41 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

44. The method according to claim 43 where the compound of formula Ia is selected from the group consisting of
(1E)-1-(4-fluorophenyl)-4-(4-pyridin-2ylpiperazin-1-yl)butan-1-one oxime;
(1Z)-1-(4-fluorophenyl)-4-(4-pyridin-2ylpiperazin-1-yl)butan-1-one oxime;
(1E)-1-(4-fluorophenyl)-4-(4-pyridin-2ylpiperazin-1-yl)butan-1-one methyloxime; and
(1E)-1-(4-fluorophenyl)-4-(4-pyridin-2ylpiperazin-1-yl)butan-1-one methyloxime.

45. The method according to claim 41 wherein
X is O;
R₂ is arylalkyl;
Z is N;
— is absent; and
R₄ is heteroaryl.

46. The method according to claim 41 wherein
X is O;
R₂ is arylalkyl wherein the arylalkyl is benzyl;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

47. The method according to claim 41 wherein
X is O;
R₂ is heteroaryl;
Z is N;
— is absent; and
R₄ is heteroaryl.

48. The method according to claim 41 wherein
X is O;
R₂ is heteroaryl wherein the heteroaryl is pyridin-3-yl;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

49. The method according to claim 41 wherein
X is O;
R₂ is aryl;
Z is C;
— is a single bond; and
R₄ is heteroaryl.

50. The method according to claim 41 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is C;
— is a single bond; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

51. The method according to claim 41 wherein
X is O;
R₂ is aryl;
Z is CH;
— is absent; and
R₄ is heteroaryl.

52. The method according to claim 41 wherein
X is O;
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is CH;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

53. The method according to claim 41 wherein
X is NR_{A};
R₂ is aryl;
Z is N;
— is absent; and
R₄ is heteroaryl.

54. The method according to claim 41 wherein
X is NR_{A};
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is N;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

55. The method according to claim 41 wherein
X is NR_{A};
R₂ is aryl;
Z is CH;
— is absent; and
R₄ is heteroaryl.

56. The method according to claim 41 wherein
X is NR_{A};
R₂ is aryl wherein the aryl is selected from the group consisting of naphthyl and phenyl wherein the phenyl is substituted with 0, 1, or 2 substituents independently selected from the group consisting of alkyl, cyano, and halogen;
Z is CH;
— is absent; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

57. The method according to claim 41 wherein R₃ is

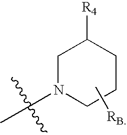

58. The method according to claim 57 wherein
R₂ is aryl; and
R₄ is heteroaryl.

59. The method according to claim 57 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

60. The method according to claim 40 wherein R₃ is

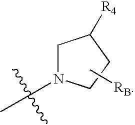

61. The method according to claim 60 wherein
R₂ is aryl; and
R₄ is heteroaryl.

62. The method according to claim 60 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is heteroaryl wherein the heteroaryl is selected from the group consisting of pyrazin-2-yl, 3-cyanopyridin-2-yl, 5-hydroxypyridin-2-yl, 3-methylpyridin-2-yl, pyridin-2-yl, 2-pyridinium N-oxide, pyrimidin-2-yl, and thiazol-2-yl.

63. A pharmaceutical composition comprising a therapeutically effective amount of a compound of formula (Ia) in combination with a pharmaceutically acceptable carrier.

64. A method of treating sexual dysfunction in a mammal comprising administering to the mammal a therapeutically effective amount of a compound of formula (I) or a pharmaceutically acceptable salt or prodrug thereof in combination with a pharmaceutically acceptable carrier.

65. A method of treating sexual dysfunction in a mammal comprising administering to the mammal a therapeutically effective amount of a compound of formula (Ia) or a pharmaceutically acceptable salt or prodrug thereof in combination with a phosphodiesterase 5 inhibitor.

66. A method of treating sexual dysfunction in a mammal comprising administering to the mammal a therapeutically effective amount of a compound of formula (Ia) or a pharmaceutically acceptable salt or prodrug thereof in combination with an adrenergic receptor antagonist.

67. A method of treating sexual dysfunction in a mammal comprising administering to the mammal a therapeutically effective amount of a compound of formula (Ia) or a pharmaceutically acceptable salt or prodrug thereof in combination with a dopamine agonist.

68. A method of treating male erectile dysfunction in a mammal comprising administering to the mammal in need of such treatment a therapeutically effective amount of a compound of formula (Ia or a pharmaceutically acceptable salt or prodrug thereof.

69. A method of treating female sexual dysfunction in a mammal comprising administering to the mammal in need of such treatment a therapeutically effective amount of a compound of formula (Ia) or a pharmaceutically acceptable salt or prodrug thereof.

70. A method of treating cardiovascular disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (Ia) or a pharmaceutically acceptable salt or prodrug thereof.

71. A compound of formula (II) 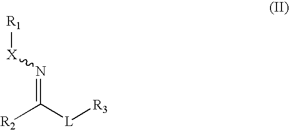
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is selected from the group consisting of 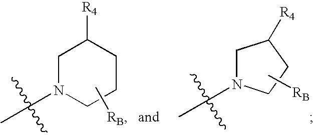
R₄ is aryl;
L is alkylene substituted with 0 or 1 substituent selected from the group consisting of alkoxy, alkoxyamino, hydroxy, and hydroxyiminoaryl; and
R_{B} is selected from the group consisting of hydrogen and alkyl.

72. The compound according to claim 70 wherein R₃ is

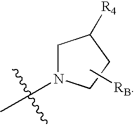

73. The compound according to claim 72 wherein
R₂ is aryl; and
R₄ is aryl.

74. The compound according to claim 72 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is aryl wherein the aryl is phenyl substituted with 0 or 1 substituent selected from the group consisting of alkoxy, cyano, and haloalkyl.

75. The compound according to claim 74 selected from the group consisting of
(1E)-1-(4-fluorophenyl)-3-{3-[3-(trifluoromethyl)phenyl]pyrrolidin-1-yl}propan-1-one O-methyloxime;
(1Z)-1-(4-fluorophenyl)-3-{3-[3-(trifluoromethyl)phenyl]pyrrolidin-1-yl}propan-1-one O-methyloxime;
1-(4-fluorophenyl)-3-[3-(2-methoxyphenyl)pyrrolidin-1-yl]propan-1-one O-methyloxime;
(1E)-1-(4-fluorophenyl)-3-[3-(3-methoxyphenyl)pyrrolidin-1-yl]propan-1-one O-methyloxime;
(1Z)-1-(4-fluorophenyl)-3-[3-(3-methoxyphenyl)pyrrolidin-1-yl]propan-1-one O-methyloxime;
(1E)-1-(4-fluorophenyl)-3-[3-(4-methoxyphenyl)pyrrolidin-1-yl]propan-1-one O-methyloxime; and
(1Z)-1-(4-fluorophenyl)-3-[3-(4-methoxyphenyl)pyrrolidin-1-yl]propan-1-one O-methyloxime.

76. The compound according to claim 70 wherein R₃ is

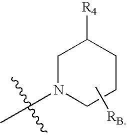

77. The compound according to claim 76 wherein
R₂ is aryl; and
R₄ is aryl.

78. The compound according to claim 76 wherein
R₂ is aryl wherein the aryl is selected from the group consisting of naphthyl and phenyl wherein the phenyl is substituted with 0, 1, or 2 substituents independently selected from the group consisting of alkyl, cyano, and halogen; and
R₄ is aryl wherein the aryl is phenyl substituted with 0 or 1 substituent selected from the group consisting of alkoxy, cyano, and haloalkyl.

79. The compound according to claim 78 selected from the group consisting of
1-(4-fluorophenyl)-3-(3-phenylpiperidin-1-yl)propan-1-one O-methyloxime;
1-phenyl-3-(3-phenylpiperidin-1-yl)propan-1-one O-methyloxime; and
1-(4-chlorophenyl)-3-(3-phenylpiperidin-1-yl)propan-1-one O-methyloxime.

80. A pharmaceutical composition comprising a therapeutically effective amount of a compound of formula (II) in combination with a pharmaceutically acceptable carrier.

81. A method of treating sexual dysfunction in a mammal comprising administering to the mammal a therapeutically effective amount of a compound of formula (II) or a pharmaceutically acceptable salt or prodrug thereof in combination with a pharmaceutically acceptable carrier.

82. A method of treating sexual dysfunction in a mammal comprising administering to the mammal a therapeutically effective amount of a compound of formula (II) or a pharmaceutically acceptable salt or prodrug thereof in combination with a phosphodiesterase 5 inhibitor.

83. A method of treating sexual dysfunction in a mammal comprising administering to the mammal a therapeutically effective amount of a compound of formula (II) or a pharmaceutically acceptable salt or prodrug thereof in combination with an adrenergic receptor antagonist.

84. A method of treating sexual dysfunction in a mammal comprising administering to the mammal a therapeutically effective amount of a compound of formula (II) or a pharmaceutically acceptable salt or prodrug thereof in combination with a dopamine agonist.

85. A method of treating male erectile dysfunction in a mammal comprising administering to the mammal in need of such treatment a therapeutically effective amount of a compound of formula (II) or a pharmaceutically acceptable salt or prodrug thereof.

86. A method of treating female sexual dysfunction in a mammal comprising administering to the mammal in need of such treatment a therapeutically effective amount of a compound of formula (II) or a pharmaceutically acceptable salt or prodrug thereof.

87. A method of treating cardiovascular disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (II) or a pharmaceutically acceptable salt or prodrug thereof.

88. A method of treating sexual dysfunction in a mammal comprising administering to said mammal in need of such treatment a therapeutically effective amount of a compound of formula (III) 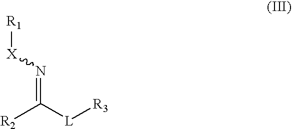
or a pharmaceutically acceptable salt or prodrug thereof, wherein
X is selected from the group consisting of O and NR_{A};
R_{A} is selected from the group consisting of hydrogen and alkyl;
R₁ is selected from the group consisting of hydrogen, alkenyl, alkoxyalkyl, alkyl, alkynyl, arylalkyl, cyanoalkyl, cycloalkyl, haloalkyl, and hydroxyalkyl;
R₂ is selected from the group consisting of aryl, arylalkyl, heteroaryl, and heteroarylalkyl;
R₃ is 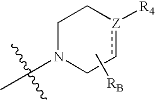
R₄ is aryl;
L is alkylene substituted with 0 or 1 substituent selected from the group consisting of alkoxy, alkoxyamino, hydroxy, and hydroxyiminoaryl;
R_{B} is selected from the group consisting of hydrogen and alkyl;
Z is selected from the group consisting of C and CH; and
— is absent or a single bond provided that when Z is C then — is a single bond.

89. A pharmaceutical composition comprising a therapeutically effective amount of a compound of formula (III) in combination with a pharmaceutically acceptable carrier.

90. A method of treating sexual dysfunction in a mammal comprising administering to the mammal a therapeutically effective amount of a compound of formula (III) or a pharmaceutically acceptable salt or prodrug thereof in combination with a pharmaceutically acceptable carrier.

91. A method of treating sexual dysfunction in a mammal comprising administering to the mammal a therapeutically effective amount of a compound of formula (III) or a pharmaceutically acceptable salt or prodrug thereof in combination with a phosphodiesterase 5 inhibitor.

92. A method of treating sexual dysfunction in a mammal comprising administering to the mammal a therapeutically effective amount of a compound of formula (III) or a pharmaceutically acceptable salt or prodrug thereof in combination with an adrenergic receptor antagonist.

93. A method of treating sexual dysfunction in a mammal comprising administering to the mammal a therapeutically effective amount of a compound of formula (III) or a pharmaceutically acceptable salt or prodrug thereof in combination with a dopamine agonist.

94. A method of treating male erectile dysfunction in a mammal comprising administering to the mammal in need of such treatment a therapeutically effective amount of a compound of formula (III) or a pharmaceutically acceptable salt or prodrug thereof.

95. A method of treating female sexual dysfunction in a mammal comprising administering to the mammal in need of such treatment a therapeutically effective amount of a compound of formula (III) or a pharmaceutically acceptable salt or prodrug thereof.

96. A method of treating cardiovascular disorders, inflammatory disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (III) or a pharmaceutically acceptable salt or prodrug thereof.
